(19) United States
(12) Patent Application Publication
(10) Pub. No.: US 2025/0388640 A1
(43) Pub. Date: Dec. 25, 2025

(54) HUMAN IL-12P40 VARIANTS AND USES THEREOF

(71) Applicant: Synthekine, Inc., Menlo Park, CA (US)

(72) Inventors: Scott Mccauley, Menlo Park, CA (US); Andrew Morin, Menlo Park, CA (US); Michael Totagrande, San Leandro, CA (US)

(21) Appl. No.: 18/702,618

(22) PCT Filed: Oct. 20, 2022
(86) PCT No.: PCT/US2022/078439

§ 371 Date: Apr. 18, 2024

Related U.S. Application Data
(60) Provisional application No. 63/257,942, filed on Oct. 20, 2021.

(51) Int. Cl. C07K 14/54; A61K 38/00

(52) U.S. Cl. CPC C07K 14/5434; A61K 38/00; C07K 2319/30; C07K 2319/31

(57) ABSTRACT

Provided herein are methods and compositions for modulating IL-12 and IL-23 signaling.

### CROSS-REFERENCE TO RELATED PATENT APPLICATIONS

The present application is a U.S. National Stage of International Application No. PCT/US2022/078439, filed Oct. 20, 2022, which claims benefit of priority to U.S. Provisional Patent Application No. 63/257,942, filed Oct. 20, 2021, both of which are incorporated by reference for all purposes.

### REFERENCE TO AN ELECTRONIC SEQUENCE LISTING

0.1] The contents of the electronic sequence listing (106249-1351147-004010PC.xml; Size: 222,201 bytes; and Date of Creation: Feb. 28, 2023) is herein incorporated by reference in its entirety.

### BACKGROUND OF THE INVENTION

Cytokine and growth-factor ligands typically signal through multimerization of cell surface receptors subunits. In some instance, cytokines act as multispecific (e.g., bispecific or trispecific) ligands which facilitate the association of such receptor subunits, bringing their intracellular domains into proximity such that intracellular signaling may occur. The nature of the cytokine determines which receptor subunits are associated to form the cytokine receptor complex. Cytokines thus act to bridge the individual receptor subunits into a receptor complex that results in intracellular signaling.

The intracellular domains of cytokine receptor subunits possess proline rich JAK binding domains which are typically located in the box1/box region of the intracellular domain of the cytokine receptor subunit near the interior surface of the cell membrane. Intracellular JAK kinases associate with JAK binding domains. When the intracellular domains receptor subunits are brought into proximity, typically by the binding of the cognate ligand for the receptor to the extracellular domains of the receptor subunits, the JAKs phosphorylate each other. Four Janus kinases have been identified in mammalian cells: JAK1, JAK2, JAK3 and TYK2. Ihle, et al. (1995) Nature 377 (6550): 591-4, 1995; O'Shea and Plenge (2012) Immunity 36 (4): 542-50. The phosphorylation of the JAK induces a conformational change in the JAK providing the ability to further phosphorylate other intracellular proteins which initiates a cascade that results in activation of multiple intracellular factors which transduce the intracellular signal associated with the receptor, resulting intracellular responses such as gene transcription, frequently referred to as downstream signaling. In many instances, the proteins which are phosphorylated by the JAKs are members of the signal transducer and activator of transcription (STAT) protein family. Seven members of the mammalian STAT family have been identified to date: STAT1, STAT2, STAT3, STAT4, STAT5a STAT5b, and STAT6. Delgoffe, et al., (2011) Curr Opin Immunol. 23 (5): 632-8; Levy and Darnell (2002) Nat Rev Mol Cell Biol. 3 (9): 651-62 and Murray, (2007) J Immunol. 178 (5): 2623-9. The selective interplay of activated JAK and STAT proteins, collectively referred to as the JAK/STAT pathway, provide for a wide variety of intracellular responses observed in response to cytokine binding.

Human IL-12 (hIL-12) is a heterodimeric cytokine comprised of the p35 and p40 subunits. hIL-12 is produced by dendritic cells, macrophages and neutrophils. The hIL-12 heterodimer is also referred to as p70. hIL-12 is typically identified as a T cell stimulating factor which can stimulate the proliferation and activity of T cells. hIL 12 stimulates the production of IFNγ and TNFα and modulates the cytotoxic activity of NK and CD8+ cytotoxic T cells. hIL-12 is also involved in the immune cell differentiation in particular the differentiation of naïve T cells into Th1 (CD4+) cells. hIL-12 is also reported to provide anti-antiangiogenic activity. hIL-12 has been proposed for use in the treatment of a variety of neoplastic diseases, viral and bacterial infections. hIL-12 binds to the hIL-12 receptor, a heterodimeric complex of hIL 12 receptor subunit beta-1 (IL-12Rβ1, also referred to in the scientific literature as IL-12Rβ1 or CD212) and hIL-12 receptor subunit beta-2 (IL-12Rβ2 also referred to in the scientific literature as IL-12Rβ2). hIL12Rβ1 and hIL12Rβ2 are members of the class I cytokine receptor family and have homology to gp130. The expression of hIL 12Rβ1 and hIL 12Rβ2 are upregulated in response to hIL-12 with the majority of hIL 12Rβ2 is found on activated T cells.

hIL12Rβ1 is a constitutively expressed type I transmembrane protein that belongs to the hemopoietin receptor superfamily. hIL12Rβ1 binds with low affinity to hIL-12. hIL12R$1 is required for high-affinity binding to the hIL-12p40 subunit and it is associated with the Janus kinase (Jak) family member Tyk-2. The binding IL 12p40 and IL12p35 to IL12Rβ1 and IL12Rβ2, respectively results in the activation of the Tyk-2 and Jak-2 Janus kinases. The phosphorylated intracellular signaling domain of IL12Rβ2 provides a binding site for STAT4, which is phosphorylated and translocated to the nucleus regulating IFN gamma gene transcription. In addition to forming one of the components of the hIL-12 receptor, hIL-12Rβ1 is also a component of the hIL-23 receptor. The hIL-23 receptor is a heterodimer of hIL-23R and hIL-12Rβ1. hIL-23 binds hIL-23R with an affinity of 44 nM but binds to hIL-12Rβ1 with a significantly lower affinity of 2 μM. There is no apparent direct binding of hIL-23R to hIL 1-2Rβ1, the completion of the hIL-23h: IL-23R: hIL-12Rβ1 complex mediated by the initial formation of the hIL-23: hIL-23R complex which in turn binds to IL12Rβ1.

The p40 subunit of the hIL-12 and hIL-23 cytokines provides the majority of binding sites for IL-12Rβ1. In addition to forming a subunit of IL 12 and IL23, p40 alone has significant bioactivity. P40 is reported to exist as both a monomer and a disulfide linked homodimer and which has a chemo attractant role for macrophages mediated by IL12Rβ1 alone.

IL-12Rβ1 and IL-12Rβ2 are members of the class I cytokine receptor family and have homology to gp130. The expression of IL-12Rβ1 and IL-12Rβ2 are upregulated in response to IL-12 with the majority of IL-12Rβ2 found on activated T cells. In response to dimerization of IL-12R β1 and IL-12R β2, Jak-2 and Tyk-2 are transphosphorylated. Phosphorylated IL-12Rβ2 binds to and phosphorylates STAT4 which then dimerizes with another phosphorylated STAT4 molecule. The phosphorylated STAT4 homodimers dimerize and the phosphorylated STAT4 homodimers translocate to the nucleus resulting in, among other activities, the promotion of IFN-γ gene transcription. The IL-12 and IFN-γ induce the activity and proliferation of macrophages, NK cells, and T cells, which also secrete IL-12.

The present disclosure provides modified human IL-12 p40 molecules that associate with human IL-12p35 molecules (hIL-12p35) to form modified hIL-12s (i.e., hIL-12 muteins comprising a modified hIL-12p40 described herein) that retain many beneficial properties of hIL-12 but reduce its known pro-inflammatory side effects. The modified human IL-12 p40 molecules provided herein can also associate with human p19 molecules to form modified hIL-23s (i.e., hIL-23 muteins comprising a modified hIL-12p40 described herein) that retain many beneficial properties of hIL-23 but reduce its known pro-inflammatory side effects. The present disclosure provides improved variants of hIL-12p40 that can be used as anti-tumor agents or immune modulators in treating various relevant diseases, including cancers, autoimmune diseases, inflammatory disease, and infection.

### SUMMARY OF THE DISCLOSURE

The present disclosure provides compositions that are useful for modulating signal transduction mediated by human interleukin-12 (hIL-12) and human interleukin-23 (hIL-23). In particular, the disclosure provides modified hIL-12p40 polypeptides, such as variants or mutants of hIL-12p40 polypeptides, with altered binding affinity to the hIL-12 receptor or the hIL-23 receptor. In some embodiments, the hIL-12p40 polypeptides have altered binding affinity to the hIL-12Rβ1 subunit of the hIL-12 receptor or hIL-23 receptor. Also provided are hIL-12 muteins comprising a modified hIL-12p40 polypeptide and a hIL-12p35 polypeptide and hIL-23 muteins comprising a modified hIL-12p40 polypeptide. Also provided are compositions and methods useful for producing such modified hIL-12p40 polypeptides, hIL-12 muteins, and hIL-23 muteins described herein. Further provided are methods for modulating hIL-12- or IL-23-mediated signaling, and methods for the treatment or prevention of conditions associated with the perturbation of signal transduction mediated by hIL-12 or hIL-23.

In some embodiments, the compositions are partial agonists of the hIL-12 receptor. The modified hIL-12p40 polypeptides described herein provide several advantages. The cognate ligand of the hIL-12 receptor, hIL-12, a heterodimer of p40 and p35, causes hIL-12Rβ1 and hIL-12Rβ2 to come into proximity (i.e., by their simultaneous binding of hIL-12). However, when hIL-12 is used as a therapeutic in mammalian, particularly human, subjects, it may also trigger a number of adverse and undesirable effects by a variety of mechanisms including binding to hIL-12Rβ1 and hIL-12Rβ on cell types that may result in undesirable effects and/or undesired signaling in cells expressing hIL-12Rβ1 and hIL-12Rβ. The present disclosure is directed to methods and compositions that modulate the multiple effects of hIL-12 binding so that desired therapeutic signaling occurs, particularly in a desired cellular or tissue subtype, while minimizing undesired activity and/or intracellular signaling in other cellular or tissue subtypes.

In some embodiments, an hIL-12 mutein of the present disclosure comprises a modified hIL-12p40 polypeptide that provides hIL-12 intracellular signaling on desired cell types, while providing significantly less hIL-12 intracellular signaling on other undesired cell types. This is achieved, for example, by contacting the cell with an IL 12 mutein comprising a modified hIL-12p40 polypeptide with a modified binding affinity for hIL-12Rβ1 or causing different Emax for hIL-12Rβ1 as compared to the binding affinity of wild-type or parental hIL-12p40 polypeptide for hIL-12Rβ1. Because different cell types respond to the binding of cognate ligands to their cognate receptors with different sensitivity, by modulating the affinity of the heterodimeric hIL-12 ligand (or its individual components) for the hIL-12 receptor (or its individual components) relative to wild-type hIL-12 (i.e., comprising wild-type p35 and p40) binding facilitates the stimulation of desired activities while reducing undesired activities on non-target cells.

Provided herein is a modified human IL-12p40 (hIL-12p40) polypeptide comprising two or more amino acid substitutions, wherein the polypeptide comprises amino acid substitutions at positions corresponding to amino acid residues E81 and F82 of SEQ ID NO: 1, wherein (a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or (b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y).

In some embodiments, the modified hIL-12p40 polypeptide is a modified human hIL-12p40 polypeptide having at least 70% sequence identity to SEQ ID NO:1 (e.g., at least 70%, 75%, 80%, 85%, 90%, 91%, 92%, 93%, 94%, 95%, 96%, 97%, 98%, or 99% sequence identity to SEQ ID NO:1), comprising two or more amino acid substitutions at positions corresponding to amino acid residues E81 and F82 of SEQ ID NO: 1, wherein (a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or (b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y).

In some embodiments, the modified hIL-12p40 polypeptide is a modified hIL-12p40 polypeptide having at least 70% sequence identity to a modified hIL-12p40 polypeptide sequence of Table 7, i.e., selected from the group consisting of SEQ ID NO: 7-SEQ ID NO: 45. (e.g., at least 70%, 75%, 80%, 85%, 90%, 91%, 92%, 93%, 94%, 95%, 96%, 97%, 98%, or 99% sequence identity to a sequence selected from the group consisting of SEQ ID NO: 7-SEQ ID NO: 45).

In some embodiments, the modified hIL-12p40 polypeptide is a modified hIL-12p40 polypeptide having at least 70% sequence identity to a modified hIL-12p40 polypeptide sequence of Table 7, i.e., selected from the group consisting of SEQ ID NO: 151-SEQ ID NO: 190. (e.g., at least 70%, 75%, 80%, 85%, 90%, 91%, 92%, 93%, 94%, 95%, 96%, 97%, 98%, or 99% sequence identity to a sequence selected from the group consisting of SEQ ID NO: 151-SEQ ID NO: 190).

In some embodiments, the modified hIL 12p40 polypeptide further comprises one or more amino acid substitutions at one or more positions corresponding to the amino acid residues selected from the group consisting of W37, P39, D40, A41, Q64, K80, A85, E108, D115, H216, K217, L218, and K219 of SEQ ID NO: 1, wherein the amino acid substitution at each position is independently selected from the twenty amino acids listed in Table 1.

Also provided is a human IL12 mutein comprising a hIL 12p40 polypeptide comprising one or more amino acid substitutions at one or more positions W37, P39, D40, A41, Q64, K80, A85, E108, D115, H216, K217, L218, and K219 of SEQ ID NO: 1, wherein the human IL12 mutein: (i) induces hIL-12 signaling in CD8+ T cells; and (ii) has decreased (for example, at least about a 10%, 20%, 30%, 40%, 50%, 60%, or 70% decreased) hIL-12 signaling in NK cells compared to a wildtype hIL-12 comprising a hIL-12p40 polypeptide lacking the one or more amino acid substitutions.

Further provided is a hIL12p40 polypeptide, wherein upon association with hIL-12p35, the polypeptide forms a dimer that activates interferon gamma (IFNγ) in CD8+ T cells and has decreased IFNγ signaling in CD8+ T cells, for example, at least about a 10%, 20%, 30%, 40%, 50%, 60%, or 70% decrease, compared to the wild-type hIL-12p40 polypeptide lacking the two or more amino acid substitutions.

Also provided is a hIL12p40 polypeptide comprising two or more amino acid substitutions at positions W37, P39, D40, A41, Q64, K80, A85, E108, D115, H216, K217, L218, and K219 of SEQ ID NO: 1, wherein upon association with hIL12p35, the hIL12p40 polypeptide forms a dimer that has a reduced binding affinity, for example, at least about a 10%, 20%, 30%, 40%, 50%, 60%, or 70% reduction, for hIL-12Rβ1 compared to the binding affinity of a wildtype hIL-12p40 polypeptide lacking the two or more amino acid substitutions. In some embodiments hIL12p40 polypeptide comprising the two or more amino acid substitutions, wherein, upon association with hIL 12p35, the polypeptide forms a dimer that has decreased STAT-4 mediated signaling, for example, at least about a 10%, 20%, 30%, 40%, 50%, 60%, or 70% decrease, compared to wildtype hIL-12p40 polypeptide lacking the two or more amino acid substitutions. In some embodiments, the hIL 12p40 polypeptide comprising the two or more amino acid substitutions, upon association with hIL 12p35, the polypeptide forms a dimer that has decreased STAT-4 mediated signaling in NK cells as compared to STAT-4 mediated signaling in CD8+ T cells.

Also provided is a nucleic acid molecule comprising a nucleic acid sequence encoding a modified hIL-12p40 polypeptide as disclosed herein. In some embodiments, the nucleic acid sequence further encodes a signal peptide 5′ to the nucleic acid sequence encoding the modified hIL-12p40 polypeptide. In some embodiments, the nucleic acid sequence further encodes a peptide linker.

In some embodiments, the present disclosure provides an expression cassette comprising a nucleic acid sequence encoding the modified hIL-12p40 polypeptide of the present disclosure operably linked to one or more heterologous nucleic acid sequences. In some embodiments, the heterologous nucleic acid sequence is an expression control sequence. In some embodiments, the expression control sequence is functional in a mammalian cell.

Also provided is a vector comprising an expression cassette nucleic acid sequence encoding a modified hIL-12p40 polypeptide disclosed herein operably linked to one or more heterologous nucleic acid sequences. In some embodiments, the vector is an expression vector. In some embodiments, the vector is viral vector. In some embodiments, the vector is non-viral vector.

In some embodiments, the present disclosure provides a vector comprising a first expression cassette comprising a nucleic acid sequence encoding p35 (SEQ ID NO: 3) operably to one or more heterologous nucleic acid sequences and the same or a second vector comprising a second expression cassette comprising a nucleic acid sequence modified hIL-12p40 polypeptide of the present disclosure operably to one or more heterologous nucleic acid sequences.

In some embodiments, the present disclosure provides a vector comprising a first expression cassette comprising a nucleic acid sequence encoding a human p19 polypeptide (SEQ ID NO: 5) operably to one or more heterologous nucleic acid sequences and the same or a second vector comprising a second expression cassette comprising a nucleic acid sequence modified hIL-12p40 polypeptide of the present disclosure operably to one or more heterologous nucleic acid sequences.

Further provided is a recombinantly modified cell comprising a nucleic acid molecule or vector of the disclosure. In some embodiments, the cell is a prokaryotic cell, such as a bacterial cell. In some embodiments, the cell is a eukaryotic cell, such as a mammalian cell. Also provided is a cell culture comprising at least one recombinantly modified cell of the disclosure, and a culture medium.

The present disclosure further provides methods for the recombinant production, isolation, purification and characterization of a modified hIL-12p40 polypeptide described herein. Thus, provided herein is a method for producing a modified hIL-12p40 polypeptide of the disclosure. In some embodiments, the method comprises a) providing one or more recombinantly modified cells comprising a nucleic acid molecule or vector comprising a nucleic acid sequence encoding a modified hIL-12p40 disclosed herein; and b) culturing the one or more cells in a culture medium such that the cells produce the modified hIL-12p40 polypeptide encoded by the nucleic acid sequence. In some embodiments, the method further comprises the step of (c) isolating and/or purifying the modified hIL-12p40 polypeptide. Also provided is a modified hIL-12p40 polypeptide produced by the above method.

Also provided is a method for producing a hIL-12 mutein (i.e., a hIL-12 heterodimer comprising a p35 polypeptide and a mutant hIL-12p40 polypeptide of the disclosure). In some embodiments, the method comprises a) providing one or more recombinantly modified cells comprising a nucleic acid molecule or vector comprising a nucleic acid sequence encoding a modified hIL-12p40 disclosed herein; and b) culturing the one or more cells in a culture medium such that the cells produce the hIL-12 mutein comprising the modified hIL-12p40 polypeptide encoded by the nucleic acid sequence. In some embodiments, the method further comprises the step of (c) isolating and/or purifying the hIL-12 mutein. Also provided is a hIL-12 mutein produced by the above method.

Any of the production methods provided herein can further comprise modifying a produced hIL-12p40 polypeptide or hIL-12 mutein to increase half-life, i.e., provide prolonged duration of action in vivo in a mammalian subject and pharmaceutically acceptable formulations thereof. In some embodiments, monomers or dimers comprising a modified hIL-12p40 polypeptide are conjugated or fused to one or more carrier molecules. In some embodiments, the carrier molecule is a protein carrier molecule. In some embodiments, the protein carrier molecule is an Fc polypeptide (for example, an Fc antibody fragment) or an albumin polypeptide.

Also provided is a pharmaceutical composition comprising a hIL 12 mutein of the present disclosure. In some embodiments, the pharmaceutical composition comprises a hIL12 mutein of the present disclosure and a pharmaceutically acceptable carrier. In some embodiments, the pharmaceutical composition comprises a nucleic acid molecule or vector of the disclosure. In some embodiments, the pharmaceutical composition comprises a recombinantly modified cell of the disclosure. In some embodiments, the recombinantly modified cell is a mammalian cell.

In another aspect, the disclosure provides a method for modulating hIL-12-mediated signaling in a subject, the method comprising administering to the subject an effective amount of a pharmaceutical composition described herein. In some embodiments, the hIL-12-mediated signaling comprises STAT4-mediated signaling. In some embodiments, the STAT4-mediated signaling is determined by an assay selected from the group consisting of a gene expression assay, a phospho-flow signaling assay, and an enzyme-linked immunosorbent assay (ELISA). In some embodiments, the STAT4-mediated signaling in the subject is reduced by about 20% to about 100% compared to a reference level. In some embodiments, the administered composition results in a reduced capacity to induce expression of IFN-γ.

The modified hIL 12p40 polypeptides of the present disclosure are useful in the treatment and/or prevention of disease in mammalian subjects. Thus, in another aspect, the disclosure provides a method for treating a health condition in a subject in need thereof, the method comprising administering to the subject a therapeutically effective amount of composition comprising: a modified hIL12p40 polypeptide, hIL-12 or IL-23 mutein described herein; a nucleic acid molecule or vector comprising a nucleic acid sequence encoding a modified hIL12p40 polypeptide described herein; a recombinantly modified cell comprising a nucleic acid molecule or vector described herein; or a pharmaceutical composition described herein. In another aspect, the disclosure provides a method of treating a neoplastic, infectious or autoimmune disease in a subject in need thereof, the method comprising administering to the subject a therapeutically effective amount of a modified hIL 12p40 polypeptide, hIL-12 mutein, IL-23 mutein, a nucleic acid molecule or vector comprising a nucleic acid sequence encoding a modified hIL 12p40 polypeptide described herein; a recombinantly modified cell comprising a nucleic acid molecule or vector described herein or a pharmaceutical composition described herein.

In some embodiments, the present disclosure provides for the treatment or prevention of an autoimmune disease in a mammalian subject by the administration of a therapeutically effective amount of a modified hIL 12p40 polypeptide or hIL-23 mutein of the present disclosure. In some embodiments, the present disclosure provides for the treatment or prevention of neoplastic disease in a mammalian subject by the administration of a therapeutically effective amount of a modified hIL 12p40 polypeptide or hIL-12 mutein of the present disclosure. In some embodiments, the present disclosure provides for the treatment or prevention of neoplastic disease in a mammalian subject by the administration of a therapeutically effective amount of a modified hIL 12p40 polypeptide or hIL-12 mutein of the present disclosure in combination with one or more supplementary therapeutic agents. In some embodiments, the modified hIL-12p40 polypeptide administered to the mammalian subject as a monomer or as part of a dimer, i.e., a hIL-12 mutein.

Also provided is a kit for modulating hIL-12-mediated or hIL-23 signaling in a subject, or treating a health condition in a subject in need thereof. In some embodiments, the kit comprises a modified hIL-12p40 polypeptide monomer an hIL-12 mutein or an hIL-23 mutein described hereinIn some embodiments, the kit comprises a nucleic acid molecule or vector comprising a nucleic acid sequence encoding a modified hIL-12p40 polypeptide described herein, a nucleic acid molecule or vector comprising a nucleic acid sequence encoding a modified hIL-12p40 polypeptide and a hIL-12 p35 polypeptide described herein, or a nucleic acid molecule or vector comprising a nucleic acid sequence encoding a modified hIL-12p40 polypeptide and a human p19 polypeptide described herein. In some embodiments the kit comprises a recombinantly modified cell comprising a nucleic acid molecule or vector described herein, or a pharmaceutical composition described herein.

### BRIEF DESCRIPTION OF THE DRAWINGS

FIGS. 1A and 1B show IFNγ production by representative hIL-12p40 mutants (E81A/F82A/K106K; E81S/F82A/K106K; E81N/F82A/K106K, E81G/F82A/K106K) of the E81X panel described in the Examples, as compared to IFNγ production by a E81A/F82A/K106A hIL-12p40 mutant).

FIGS. 2A-2D show STAT4 activity by representative hIL-12p40 mutants (E81A/F82Y/K106K; E81A/F82A/K106K; E81A/F82A/K106K; E81A/F82M/K106K; E81A/F82F/K106K) of the F82X panel described in the Examples, in CD8+ T cells (FIGS. 2A-2B) and NK cells (FIGS. 2C-2D), as compared to a E81E/F82F/K106K hIL-12p40 polypeptide (wildtype).

FIG. 3 is a diagram depicting an exemplary “knob into hole” hIL-12 mutein-Fc fusion construct.

FIGS. 4A-K show the results of a HEK293-BLUE Human IL12 pSTAT4 Reporter Assay for a series of human IL-12 muteins comprising modified human IL-12 p40 polypeptides wherein E81 was substituted and F82 is alanine.

### DETAILED DESCRIPTION OF THE INVENTION

To facilitate the understanding of present disclosure, certain terms and phrases are defined below as well as throughout the specification. The definitions provided herein are non-limiting and should be read in view of the knowledge of one of skill in the art.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications, patents, published patent applications, GenBank accession numbers and UniProt reference numbers mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Publications cited herein and the material for which they are cited are hereby specifically incorporated by reference in their entireties.

Unless indicated otherwise, parts are parts by weight, molecular weight is weight average molecular weight, temperature is in degrees Celsius (C), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; ug=microgram; mg=milligram; g=gram; kg=kilogram; dl or dL=deciliter; μl or μL=microliter; ml or mL=milliliter; 1 or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=once weekly; QM=once monthly; HPLC=high performance liquid chromatography; BW=body weight; U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1.

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

P40 Residue Numbering: In the present disclosure, the numbering of amino acid residues of human P40 is made in reference to the number of the “pro” form of hP40 as provided in (SEQ ID NO: 1). In reference to the muteins described herein, substitutions are designated herein by the one letter amino acid code followed by the pro-hp40 (SEQ ID NO: 1) amino acid position followed by the one letter amino acid code which is substituted. For example, a mutein having the modification “E81A” refers to a substitution of the glutamic acid (E) residue at position 81 of the (SEQ ID NO: 1) with an alanine (A) residue at this position. A deletion of an amino acid residue is referred to as “des” or the symbol “A” followed by the amino acid residue and its position.

### Definitions

Unless otherwise indicated, the following terms are intended to have the meaning set forth below. Other terms are defined elsewhere throughout the specification.

The term “about” refers to a value that is plus or minus 10% of a numerical value described herein, such as plus or minus 1%, 2%, 3%, 4%, 5%, 6%, 7%, 8%, 9%, or 10% of numerical value described herein. The term “about” also applies to all numerical ranges described herein. All values described herein are understood to be modified by the term “about” whether or not the term “about” is explicitly recited in reference to a given value.

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand. The term activate is also used in reference to a cell that expresses a receptor wherein one more biological activities of the cell are modulated (e.g. upregulation or downregulation of STAT4 signaling) in response to a the binding of a ligand for such receptor.

Activity: As used herein, the term “activity” is used with respect to a molecule to describe a property of the molecule with respect to a test system (e.g., an assay) or biological or chemical property (e.g., the degree of binding of the molecule to another molecule) or of a physical property of a material or cell (e.g., modification of cell membrane potential). Examples of such biological functions include but are not limited to catalytic activity of a biological agent, the ability to stimulate intracellular signaling, gene expression, cell proliferation, and the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT3 phosphorylation]/[mg protein], [STAT4 phosphorylation]/[mg protein] [proliferation]/[mg protein], plaque forming units (pfu), etc. As used herein, the term proliferative activity refers to an activity that promotes cell proliferation and replication, including dysregulated cell division such as that observed in neoplastic diseases, inflammatory diseases, fibrosis, dysplasia, cell transformation, metastasis, and angiogenesis.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g., a modified hIL-12p40 polypeptide, hIL-12 mutein comprising a modified hIL-12p40 polypeptide or hIL-23 mutein comprising a modified hIL-12p40 polypeptide; an engineered cell expressing a modified hIL-12p40 polypeptide, hIL-12 mutein comprising a modified hIL-12p40 polypeptide, or a hIL-23 mutein comprising a modified hIL-12p40 polypeptide; or a pharmaceutical formulation comprising one or more of the foregoing), alone or in combination with one or more supplementary agents. Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), inhalation (e.g respiratory inhalers including dry-powder inhalers), intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (KD), a ratio of the dissociation rate constant between the molecule and its target (Koff) and the association rate constant between the molecule and its target (Kon).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in modulation of cell proliferation or pathways or the cell cycle. In some embodiments, an agonist is an modified form of a cognate ligand that binds to its cognate receptor and alters the state of the cognate receptor in a biological response that mimics the biological effect of the interaction of the naturally occurring cognate ligand with its cognate receptor. The term “agonist” includes partial agonists, full agonists and superagonists. An agonist may be described as a “full agonist” when such agonist which leads to a substantially full biological response (i.e. the response associated with the naturally occurring ligand/receptor binding interaction) induced by receptor under study, or a partial agonist. A “superagonist” is a type of agonist that can produce a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay. It should be noted that the biological effects associated with the full agonist may differ in degree and/or in kind from those biological effects of partial or superagonists. In contrast to agonists, antagonists may specifically bind to a receptor but do not result in the signal cascade typically initiated by the receptor and may modify the actions of an agonist at that receptor. Inverse agonists are agents that produce a pharmacological response that is opposite in direction to that of an agonist.

Antagonist: As used herein, the term “antagonist” or “inhibitor” refers to a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway including an immune checkpoint pathway, or cell.

Biological Sample: As used herein, the term “biological sample” or “sample” refers to a sample obtained (or derived) from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, tumor tissue, including immunoglobulin enriched or cell-type specific enriched fractions derived from one or more of such tissues.

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 35%, alternatively by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

Conservative Amino Acid Substitution: As used herein, the term “conservative amino acid substitution” refers to an amino acid replacement that changes a given amino acid to a different amino acid with similar biochemical properties (e.g., charge, hydrophobicity, and size). For example, the amino acids in each of the following groups are considered as conservative amino acids of each other: (1) hydrophobic amino acids: alanine, isoleucine, leucine, tryptophan, phenylalanine, valine, proline, and glycine; (2) polar amino acids: glutamine, asparagine, histidine, serine, threonine, tyrosine, methionine, and cysteine; (3) basic amino acids: lysine and arginine; and (4) acidic amino acids: aspartic acid and glutamic acid.

Corresponding To: As used herein, the terms “correspondence” or “corresponding to” in the context of an amino acid or nucleic acid sequence refers to the equivalent position of a reference sequence that is aligned with one or more other sequences to maximize the percentage of sequence identity. For example, an “amino acid position corresponding to amino acid position [X]” of a specified hIL-12p40 polypeptide refers to equivalent positions, based on alignment, in other hIL-12p40 polypeptides, including structural homologues and variants. The corresponding position can be based on a reference, wild-type or parental sequence, for example the amino acid sequence of SEQ ID NO:1.

Derived From: As used herein, the term “derived from” is used in the context of an amino acid sequence or nucleic acid to indicate that the polypeptide or nucleic acid has a sequence that is based on that of a reference polypeptide or nucleic acid and is not meant to be limiting as to the source or method by which the protein or nucleic acid is made. By way of example, the term “derived from” includes homologs or variants of reference amino acid or DNA sequences.

Effective Concentration (EC): As used herein, the terms “effective concentration” or its abbreviation “EC” are used interchangeably to refer to the concentration of an agent in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation EC is provided with a subscript (e.g., EC40, EC50, etc.) the subscript refers to the percentage of the Emax of the biological response observed at that concentration. For example, the concentration of a test agent sufficient to result in the induction of a measurable biological parameter in a test system that is 30% of the maximal level of such measurable biological parameter in response to such test agent, this is referred to as the “EC30” of the test agent with respect to such biological parameter. Similarly, the term “EC100” is used to denote the effective concentration of an agent that results in the maximal (100%) response of a measurable parameter in response to such agent. Similarly, the term EC50 (which is commonly used in the field of pharmacodynamics) refers to the concentration of an agent sufficient to result in the half-maximal (about 50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC50 is the drug concentration to give the half-maximal effect.

Enriched: As used herein in the term “enriched” refers to a sample comprising a species of interest (e.g. a molecule or cell) wherein the sample is non-naturally manipulated so that a species of interest is present in: (a) a greater concentration (e.g., at least 3-fold greater, alternatively at least 5-fold greater, alternatively at least 10-fold greater, alternatively at least 50-fold greater, alternatively at least 100-fold greater, or alternatively at least 1000-fold greater) than the concentration of the species in the starting sample, such as a biological sample (e.g., a sample in which the molecule naturally occurs or in which it is present after administration); or (b) a concentration greater than the environment in which the molecule was made (e.g., a recombinantly modified bacterial or mammalian cell).

Extracellular Domain: As used herein the term “extracellular domain” or its abbreviation “ECD” refers to the portion of a cell surface protein which is external to the plasma membrane of the cell on which it is expressed. A cell surface protein comprising and ECD may be a transmembrane protein, a cell surface or membrane associated protein that comprising a domain associated with the cell membrane but which lacks an intracellular domain . . .

Identity: The term “identity,” as used herein in reference to polypeptide or DNA sequences, refers to the subunit sequence identity between two molecules. When a subunit position in both of the molecules is occupied by the same amino acid or nucleotide then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as BLAST 2.0 algorithms, which are described in Altschul et al. (1990) J. Mol. Biol. 215:403-410 and Altschul, et al. (1977) Nucleic Acids Res. 25:3389-3402. Software for performing BLAST analyses is publicly available through the National Center for Biotechnology Information (NCBI) web site. The algorithm involves first identifying high scoring sequence pairs (HSPs) by identifying short words of length W of the query sequence, which either match or satisfy some positive-valued threshold score “T” when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (the reward score for a pair of matching residues; always >0) and “N” (the penalty score for mismatching residues; always <0). For amino acid sequences, a scoring matrix is used to calculate the cumulative score. Extension of the word hits in each direction are halted when: (a) the cumulative alignment score falls off by the quantity X from its maximum achieved value; the cumulative score goes to zero or below, due to the accumulation of one or more negative-scoring residue alignments; or (b) the end of either sequence is reached. The BLAST algorithm parameters “W”, “T”, and “X” determine the sensitivity and speed of the alignment. The BLASTN program (for nucleotide sequences) functions similarly but uses as defaults a word size (“W”) of 28, an expectation (“E”) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, (1989) PNAS (USA) 89:10915-10919).

In An Amount Sufficient Amount to Effect a Response: As used herein the phrase “in an amount sufficient to cause a response” is used in reference to the amount of an agent sufficient to provide a detectable change in the level of an indicator measured before (e.g., a baseline level) and after the application of a test agent to a test system. In some embodiments, the test system is a cell, tissue or organism. In some embodiments, the test system is an in vitro test system such as a fluorescent assay. In some embodiments, the test system is an in vivo system which involves the measurement of a change in the level a parameter of a cell, tissue, or organism reflective of a biological function before and after the application of the test agent to the cell, tissue, or organism. In some embodiments, the indicator is reflective of biological function or state of development of a cell evaluated in an assay in response to the administration of a quantity of the test agent. In some embodiments, the test system involves the measurement of a change in the level an indicator of a cell, tissue, or organism reflective of a biological condition before and after the application of one or more test agents to the cell, tissue, or organism. The term “in an amount sufficient to effect a response” may be sufficient to be a therapeutically effective amount but may also be more or less than a therapeutically effective amount.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise. In some embodiments, a subject in need of treatment has been diagnosed with a disease or condition, for example, cancer, an autoimmune disorder or an infection.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise. In some embodiments, prevention refers to reducing, forestalling or delaying the onset of a particular disease, or reducing forestalling or delaying a recurrence of a particular disease, for example, after treatment for that disease. A recurrence does not necessarily have to be after curing or remission of a disease. It is sufficient to have one or more clinical symptoms reappear after a period devoid of those symptoms, for example, a period after treatment for cancer, an autoimmune disease or an infection.

Inhibitor: As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Intracellular Domain: As used herein the term “intracellular domain” or its abbreviation “ICD” refers to the portion of a cell surface protein (e.g., a cell surface receptor) which is inside of the plasma membrane of a cell. A cell surface protein comprising an ICD may be a transmembrane protein, a cell surface or membrane associated protein that comprising a domain associated with the cell membrane but which lacks an extracellular domain. The ICD may include the entire cytoplasmic portion of a transmembrane protein or membrane associated protein, or intracellular protein. A cell surface protein may be a transmembrane protein, a cell surface or membrane associated protein that comprising a domain associated with the cell membrane but which lacks an intracellular domain . . .

Isolated: As used herein the term “isolated” is used in reference to a polypeptide of interest that, if naturally occurring, is in an environment different from that in which it naturally occurs. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was synthesized, for example isolated from a recombinant cell culture comprising cells engineered to express the polypeptide or by a solution resulting from solid phase synthetic means.

Ligand: As used herein, the term “ligand” refers to a molecule that specifically binds a receptor and causes a change in the receptor so as to effect a change in the activity of the receptor or a response in cell that expresses that receptor. In one embodiment, the term “ligand” refers to a molecule or complex thereof that can act as an agonist or antagonist of a receptor. The complex of a ligand and receptor is termed a “ligand-receptor complex” (for example, and hIL-12-hIL-12 receptor complex). In some examples, the term “cognate ligand” and “cognate receptor” are used to denote a naturally occurring ligand and the receptor to which such ligand exhibits selective binding in a naturally occurring biological systems. For example, hIL-12 is the cognate ligand for the hIL-12 receptor. In another example, hIL-23 is the cognate ligand for the hIL-23 receptor.

Modified: As used herein, the term “modified” refers to a molecule, such as a polypeptide, whose structure has been changed relative to an unmodified parental molecule. A modified polypeptide typically retains one or more activities or functions of the unmodified parental molecule. For example, a modified IL-12 p40 polypeptide can activate hIL-12 signaling in a cell expressing the hIL-12 receptor as part of a heterodimer (i.e., a p35/p40 complex), but can have improved properties relative to the unmodified polypeptide. The term modified includes amino acid substitutions that are not present in a parental or wild-type hIL-12, and includes variants and mutants of an hIL-12 p40 polypeptide.

Modulate: As used herein, the terms “modulate”, “modulation” and the like refer to the ability of an agent, for example, a test agent, to cause a response, either positive or negative or directly or indirectly, in a system, including a biological system, or biochemical pathway. The term modulator includes both agonists (including partial agonists, full agonists and superagonists) and antagonists.

Mutein: As used herein the term “mutein” refers to a variant of a naturally occurring hIL-12 (i.e., a p35/p40 complex) or hIL-23 (i.e., a p19/p40 complex), i.e., a heterodimer that retains one or more biological activities of the parent, naturally occurring heterodimeric hIL-12 or hIL-23 from which it was derived. The hIL-12 muteins and hIL-23 muteins described herein can comprise any of the modified hIL-12 p40 polypeptides described herein.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein (e.g., Emax) is diminished relative to the to the wild-type molecule, however the two are nevertheless considered operably linked. When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally, in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Parent Polypeptide: As used herein, the terms “parent polypeptide” or “parent protein” are used interchangeably to designate the source of a second polypeptide (e.g., a derivative, mutant or variant) which is modified with respect to a first “parent” polypeptide. In some instances, the parent polypeptide is a wild-type or naturally occurring form of a protein. In some instance, the parent polypeptide may be a modified form a naturally occurring protein that is further modified. The term “parent polypeptide” may refer to the polypeptide itself or compositions that comprise the parent polypeptide (e.g., glycosylated or PEGylated forms and/or fusion proteins comprising the parent polypeptide). The term parent polypeptide can also be used interchangeably with “reference polypeptide.”

Partial Agonist: As used herein, the term “partial agonist” refers to a molecule (e.g., a ligand) that specifically binds to and activates a given receptor but possesses only partial activation of the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Partial agonists can be used to activate receptors to give a desired submaximal response in a subject when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. An partial agonist may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the activity of the reference polypeptide when evaluated at similar concentrations in a given assay system.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide include fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g., antigenic diphtheria or tetanus toxin or toxoid fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof. A course of action to prevent a disease, disorder or condition in a subject is typically applied in the context of a subject who is predisposed to developing a disease, disorder or condition due to genetic, experiential or environmental factors of developing a particular disease, disorder or condition. In certain instances, the terms “prevent”, “preventing”, “prevention” are also used to refer to the slowing of the progression of a disease, disorder or condition from an existing state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a cell membrane associated protein that comprises and extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain referred to as a transmembrane domain™. The binding of a ligand to the receptor results in a conformational change in the receptor resulting in a measurable biological effect. In some instances, where the receptor is a membrane spanning polypeptide comprising an ECD, TM and ICD, the binding of a ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface receptor that is not associated with any intracellular signaling alone but upon ligand binding facilitates the formation of a heteromultimeric (including heterodimeric, heterotrimeric, etc.) or homomultimeric (including homodimeric, homotrimeric, homotetrameric, etc.) complex that results in a measurable biological effect in the cell such as activation of an intracellular signaling cascade (e.g., the Jak/STAT pathway). In some embodiments, a receptor is a membrane spanning single chain polypeptide comprising ECD, TM and ICD domains wherein the ECD, TM and ICD domains are derived from the same or differing naturally occurring receptor variants or synthetic functional equivalents thereof.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”). Similarly a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of activation of T cells in response to the administration of a test agent may be determined by flow cytometric methods. In some methods, a response can be measured by determining the level of STAT (e.g., STAT3, STAT4) phosphorylation, or IFNγ production, in accordance with methods well known in the art.

Significantly Reduced Binding: As used herein, the term “exhibits significantly reduced binding” is used with respect to a variant of a first molecule (e.g., a ligand) which exhibits a significant reduction in the affinity for a second molecule (e.g., receptor) relative to the parent form of the first molecule. With respect to variant ligands, for example, a variant hIL-12p40 polypeptide or a hIL-12 mutein described herein, a variant ligand “exhibits significantly reduced binding” if the mutein binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived.

Specifically Binds: As used herein the term “specifically binds” refers to the degree of affinity for which a first molecule exhibits with respect to a second molecule. In the context of binding pairs (e.g., ligand/receptor) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In some embodiments, where the ligand is a modified hIL-12p40 polypeptide or an hIL-12 mutein described herein, and the receptor comprises an hIL-12Rβ1, the modified hIL-12p40 polypeptide specifically binds if the equilibrium dissociation constant of modified hIL-12p40 polypeptide (or hIL-12 mutein comprising a modified hIL-12p40 polypeptide/IL12R β1 ECD is greater than about 10⁻⁵ M, alternatively greater than about 10-6 M, alternatively greater than about 107 M, alternatively greater than about 10-8 M, alternatively greater than about 10-9 M, alternatively greater than about 10-10 M, or alternatively greater than about 10-11 M. Specific binding may be assessed using techniques known in the art including but not limited to competition ELISA assays, radioactive ligand binding assays (e.g., saturation binding, Scatchard plot, nonlinear curve fitting programs and competition binding assays); non-radioactive ligand binding assays (e.g., fluorescence polarization (FP), fluorescence resonance energy transfer (FRET); liquid phase ligand binding assays (e.g., real-time polymerase chain reaction (RT-qPCR), and immunoprecipitation); and solid phase ligand binding assays (e.g., multiwell plate assays, on-bead ligand binding assays, on-column ligand binding assays, and filter assays)) and surface plasmon resonance assays (see, e.g., Drescher et al., (2009) Methods Mol Biol 493:323-343 with commercially available instrumentation such as the Biacore 8K, Biacore 8K+, Biacore S200, Biacore T200 (Cytiva, 100 Results Way, Marlborough MA 01752).

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95% of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95% of the total content of the composition comprising the protein.

Suffering From: As used herein, the term “suffering from” refers to a determination made by a physician with respect to a subject based on the available objective or subjective information accepted in the field for the identification of a disease, disorder or condition including but not limited to X-ray, CT-scans, conventional laboratory diagnostic tests (e.g., blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

T-cell: As used herein the term “T-cell” or “T cell” is used in its conventional sense to refer to a lymphocytes that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8+ T cells, cytotoxic CD8+ T cells, naïve CD4+ T cells, helper T cells, e.g., TH1, TH2, TH9, TH11, TH22, TFH; regulatory T cells, e.g., TRI, Tregs, inducible Tregs; memory T cells, e.g., central memory T cells, effector memory T cells, NKT cells, tumor infiltrating lymphocytes (TILs) and engineered variants of such T-cells including but not limited to CAR-T cells, recombinantly modified TILs and TCR-engineered cells. In some embodiments the T cell is a T cell expressing the IL 12 receptor referred to interchangeably as IL12R cell, IL12R+ cell, IL 12R T cell, or IL12R+T cell.

Terminus/Terminal: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide. As uned herein in the context of nucleic acids, the “5′-terminus” (or “five-prime terminus”) and “3′-terminus” (or “carboxyl terminus”) refer to the extreme ends of the nucleic acid sequence, respectively, while the terms “5′” and “3′” refer to relative positions in the nucleic acid sequence of the polypeptide toward the 5′-terminus and the 3′-terminus, respectively, and can include the residues at the 5′-terminus and 3′-terminus, respectively.

Therapeutically Effective Amount: As used herein to the phrase “therapeutically effective amount” refers to the quantity of an agent when administered to a subject, either alone or as part of a pharmaceutical composition or treatment regimen, in a single dose or as part of a series of doses, provides a positive effect on any quantitative or qualitative symptom, aspect, or characteristic of a disease, disorder or condition. A therapeutically effective amount can be ascertained by measuring relevant physiological effects, and it may be adjusted in connection with a dosing regimen and in response to diagnostic analysis of the subject's condition. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. In one embodiment, a therapeutically effective amount is an amount of an agent when used alone or in combination with another agent provides an provides a positive effect on any quantitative or qualitative symptom, aspect, or characteristic of a disease, disorder or condition and does not result in non-reversible serious adverse events in the course of administration of the agent to the mammalian subject.

Treat: The terms “treat”, “treating”, treatment” and the like refer to a course of action (such as contacting the subject with pharmaceutical composition comprising a hIL-12 mutein alone or in combination with a supplementary agent) that is initiated with respect to a subject in response to a diagnosis that the subject is suffering from a disease, disorder or condition, or a symptom thereof, the course of action being initiated so as to eliminate, reduce, suppress, mitigate, or ameliorate, either temporarily or permanently, at least one of: (a) the underlying causes of such disease, disorder, or condition afflicting a subject; and/or (b) at least one of the symptoms associated with such disease, disorder, or condition. In some embodiments, treating includes a course of action taken with respect to a subject suffering from a disease where the course of action results in the inhibition (e.g., arrests the development of the disease, disorder or condition) or ameliorates one or more symptoms associated with the presence of the disease in the subject.

Variant: The terms “variant”, “protein variant” or “variant protein” or “variant polypeptide” are used interchangeably herein to refer to a polypeptide that differs from a parent polypeptide by virtue of at least one amino acid modification, substitution, or deletion. The parent polypeptide may be a naturally occurring or wild-type (WT) polypeptide or may be a modified version of a WT polypeptide. The term variant polypeptide may refer to the polypeptide itself, a composition comprising the polypeptide, or the nucleic acid sequence that encodes it. In some embodiments, the variant polypeptide comprises from about one to about ten, alternatively about one to about eight, alternatively about one to about seven, alternatively about one to about five, alternatively about one to about four, alternatively from about one to about three alternatively from one to two amino acid modifications, substitutions, or deletions, or alternatively a single amino acid amino acid modification, substitution, or deletion compared to the parent polypeptide. A variant may be at least about 99% identical, alternatively at least about 98% identical, alternatively at least about 97% identical, alternatively at least about 95% identical, or alternatively at least about 90% identical to the parent polypeptide from which the variant is derived.

Wild Type: By “wild type” or “WT” or “native” herein is meant an amino acid sequence or a nucleotide sequence that is found in nature, including allelic variations. A wild-type protein, polypeptide, antibody, immunoglobulin, IgG, etc. has an amino acid sequence or a nucleotide sequence that has not been modified by the hand of man.

It will be understood that individual embodiments, which are separately described herein for clarity and brevity, can be combined without limitation. Thus, the present disclosure includes one or more, or all, combinations of the embodiments described herein as if each and every combination was individually and explicitly disclosed. This also applies to any and all sub-combinations of the embodiments disclosed herein, such that the present disclosure includes one or more, or all, sub-combinations of the embodiments described herein as if each and every sub-combination was individually and explicitly disclosed.

### Compositions

Provided herein are modified hIL-12p40 polypeptides that, upon association with a hIL-12p35 polypeptide, modulate receptor binding and downstream signaling compared to a wild-type or parental hIL-12p40 polypeptide, and/or are expressed at higher levels compared to a wild-type or parental hIL-12p40 polypeptide. Also provided are hIL-12 muteins comprising a modified hIL-12p40 polypeptide described herein and a hIL-12 p35 polypeptide.

Further provided is an hIL-12 mutein wherein the hIL-12p35 and modified hIL-12p40 subunits are linked via a polypeptide linker to form a single chain hIL-12 mutein. See, for example, Lieschke, et al. “Bioactive murine and human interleukin-12 fusion proteins which retain antitumor activity in vivo,” Nature Biotechnology 15:35-40 (1997). In some embodiments, the single chain hIL-12 mutein comprises a polypeptide of the formula:

$$P {40}^{m} - {L}_{a} - P 35 or$$

$$P 35 - {L}_{a} - P {40}^{m}$$

wherein:
L linker from 1-50 amino acids,
P35 is a human p35 molecule having at least 70% sequence identity to SEQ ID NO:3 (e.g., at least 70%, 75%, 80%, 85%, 90%, 91%, 92%, 93%, 94%, 95%, 96%, 97%, 98%, 99% or 100% sequence identity to SEQ ID NO:3);
P40^{m} is a modified hIL 12p40 polypeptide described herein, for example, a modified hIL-12p40 polypeptide sequence from Table 7 (i.e., selected from the group consisting of SEQ ID NO: 7-SEQ ID NO: 46 and SEQ ID NO: 151-SEQ ID NO 190), and
a=0 (absent) or 1 (present).

Also provided are pharmaceutical compositions comprising the modified hIL-12p40 polypeptides; hIL-12 muteins comprising a modified hIL-12p40 polypeptide described herein, or hIL-23 muteins comprising a modified hIL-12p40 polypeptide described herein; recombinant nucleic acid molecules comprising a nucleic acid sequence encoding the modified hIL-12p40 polypeptides; recombinant cells engineered to express the modified hIL-12p40 polypeptides, hIL-12 muteins or hIL-23 muteins described herein. Kits comprising the modified hIL-12p40 polypeptides hIL-12 muteins comprising the modified hIL-12p40 polypeptides described herein, or hIL-23 muteins comprising the modified hIL-12p40 polypeptides described herein are also provided.

Modified hIL-12p40 Polypeptides

Provided herein are compositions comprising modified hIL-12p40 polypeptides that have improved pharmacological or therapeutic properties, and methods of using such compositions. The modified hIL-12p40 polypeptides and hIL-12 muteins comprising the modified hIL-12p40 polypeptide described herein include partial agonists that have the following advantages. First, the modified hIL-12p40 polypeptides described herein have beneficial properties, such as anti-inflammatory properties, and/or have reduced undesirable properties, such as pro-inflammatory side effects. For example, the modified hIL-12p40 polypeptide, upon association with hIL-12p35, retains the ability of wild-type hIL-12p40 to stimulate or activate IL-12 signalling in CD8+ T cells, but substantially reduces the pro-inflammatory response and IFNγ and/or STAT4-mediated signaling in NK cells. In some embodiments, the amino acid substitutions in the disclosed recombinant polypeptides results in a cell-type biased signaling of the downstream signal transduction mediated through interleukin-12 (hIL-12) compared to a reference polypeptide lacking the amino acid substitutions. In some embodiments, the cell-type biased signaling includes a reduced capability of the recombinant polypeptide to stimulate hIL-12-mediated signaling in NK cells. In some embodiments, the cell-type biased signaling includes a substantially unaltered capability of the recombinant polypeptide to stimulate hIL-12 signaling in CD8+ T cells. In some embodiments, the amino acid substitutions results in a reduced capability of the recombinant polypeptide to stimulate hIL-12 signaling in NK cells while substantially retains its capability to stimulate hIL-12 signaling in CD8+ T cells. Thus, the modified hIL-12p40 polypeptides inhibit pro-inflammatory responses and/or STAT4-mediated signaling in a cell-type dependent manner.

Second, the modified hIL-12p40 polypeptides described herein can be expressed at higher levels in cells compared to an unmodified parental polypeptide. In some embodiments, the modified hIL-12p40 polypeptides described herein provide substantial increases in yield when expressed in cells without significantly affecting the biological activity of the modified hIL-12p40 polypeptide. Thus, in some embodiments, the modified hIL-12p40 polypeptides described herein are both expressed at higher levels in cells compared to an unmodified parental polypeptide and also have desirable properties, such as anti-inflammatory properties, and/or reduced undesirable properties, such as pro-inflammatory side effects.

In some embodiments, the modified hIL-12p40 polypeptides of the disclosure are derived from a wild-type or parental hIL-12p40 polypeptide. As set forth above, human IL-12 is a non-covalently linked heterodimeric protein comprising two subunits, hIL-12p40 and hIL-12p35. The naturally occurring form of hIL 12 comprises an interchain disulfide linkage between residue C96 of p35 (numbered in accordance with SEQ ID NO: 3) and residue C199 of p40 (numbered in accordance with SEQ ID NO: 1)

The hIL-12p40 polypeptide is expressed as a 328 amino acid pre-protein comprising a 22 amino acid signal sequence (SEQ ID NO:2) which is post-translationally removed to render a 306 amino acid mature protein. Wildtype hIL-12p40 contains four intrachain disulfides between residues C50 and C90, C131 and C142, C170 and C193, and C300 and C327 (numbered in accordance with SEQ ID NO:1). The canonical amino acid sequence of the hIL-12p40 protein (UniProt Reference No. P29460) with the signal sequence (italicized) is:

(SEQ ID NO: 1)
MCHQQLVISWFSLVFLASPLVAIWELKKDVYVVELDWYPDAPGEMVVLTC
DTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSR
QVEVSWEYPDTWSTPHSYFSLTFCVQVQGKSKREKKDRVFTDKTSATVIC
RKNASISVRAQDRYYSSSWSEWASVPCS

The mature form of p40, lacking the signal sequence (SEQ ID NO:2), is:

(SEQ ID NO: 148)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCS

The hIL-12p35 monomer is expressed as a 219 amino acid pre-protein comprising a 22 amino acid signal sequence (SEQ ID NO:4) which is post-translationally removed to render a 197 amino acid mature protein. Wildtype hIL-12p35 contains two intrachain disulfide linkages, the first between residues C64 and C196 and the second between residues C85 and C123 (numbered in accordance with SEQ ID NO: 3). The canonical amino acid sequence of the hIL-12p35 protein (UniProt Reference No. P29459) with the signal sequence (italicized) is:

(SEQ ID NO: 3)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

The mature form of p35, lacking the signal sequence (SEQ ID NO:4), is:

(SEQ ID NO: 149)
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNAS

Human IL-23 is a non-covalently linked heterodimeric protein comprising two subunits, hIL-12p40 and human p19. The human p19 monomer is expressed as a 189 amino acid pre-protein comprising a 19 amino acid signal sequence (SEQ ID NO: 6) which is post-translationally removed to render a 180 amino acid mature protein. The canonical amino acid sequence of the human p19 protein (UniProt Reference No. Q9NPF7)) with the signal sequence (italicized) is:

(SEQ ID NO: 5)
MLGSRAVMLL LLLPWTAQGRAVPGGSSPAW TQCQQLSQKL
CTLAWSAHPL VGHMDLREEGDEETTNDVPH IQCGDGCDPQ
GLRDNSQFCL QRIHQGLIFY EKLLGSDIFT
GEPSLLPDSPVGQLHASLLG LSQLLQPEGH HWETQQIPSL
SPSQPWQRLL LRFKILRSLQ AFVAVAARVFAHGAATLSP

The mature form of p19, lacking the signal sequence (SEQ ID NO:6), is:

(SEQ ID NO: 150)
RAVPGGSSPAW TQCQQLSQKL CTLAWSAHPL
VGHMDLREEGDEETTNDVPH IQCGDGCDPQ GLRDNSQFCL
QRIHQGLIFY EKLLGSDIFT GEPSLLPDSPVGQLHASLLG
LSQLLQPEGH HWETQQIPSL SPSQPWQRLL
LRFKILRSLQ AFVAVAARVFAHGAATLSP

In some embodiments, the modified hIL-12p40 polypeptide is derived from a human hIL-12p40 polypeptide, such as a wild-type human hIL-12p40 polypeptide (UniProtKB-P29460; SEQ ID NO:1). In some embodiments, the modified hIL-12p40 polypeptide comprises an amino acid sequence having at least 70% sequence identity to SEQ ID NO: 1 (e.g., at least 70%, 75%, 80%, 85%, 90%, 91%, 92%, 93%, 94%, 95%, 96%, 97%, 98%, or 99% sequence identity to SEQ ID NO:1).

In some embodiments, the modified human IL-12p40 (hIL-12p40) polypeptide comprises two or more amino acid substitutions, wherein the polypeptide comprises amino acid substitutions at positions corresponding to amino acid residues E81 and F82 of SEQ ID NO: 1, wherein (a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or (b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y).

In some embodiments, the modified hIL-12p40 polypeptide is a modified human hIL-12p40 polypeptide monomer having at least 70% sequence identity to SEQ ID NO: 1 (e.g., at least 70%, 75%, 80%, 85%, 90%, 91%, 92%, 93%, 94%, 95%, 96%, 97%, 98%, or 99% sequence identity to SEQ ID NO:1), comprising two or more amino acid substitutions at positions corresponding to amino acid residues E81 and F82 of SEQ ID NO: 1, wherein (a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or (b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y).

In some embodiments, the modified hIL-12p40 polypeptide is a modified hIL-12p40 polypeptide having at least 70% sequence identity to a modified hIL-12p40 polypeptide sequence of Table 7, i.e., selected from the group consisting of SEQ ID NO: 7-SEQ ID NO: 45. (e.g., at least 70%, 75%, 80%, 85%, 90%, 91%, 92%, 93%, 94%, 95%, 96%, 97%, 98%, or 99% sequence identity to a sequence selected from the group consisting of SEQ ID NO: 7-SEQ ID NO: 45.)

In some embodiments, the modified hIL 12p40 polypeptide further comprises one or more amino acid substitutions at one or more positions corresponding to an amino acid residue selected from the group consisting of W37, P39, D40, A41, Q64, K80, A85, E108, D115, H216, K217, L218, and K219 of SEQ ID NO: 1, wherein the amino acid substitution is any amino acid.

### Receptor Binding and Intracellular Signaling

The hIL-12 receptor signaling complex comprises hIL-12Rβ1 and hIL-12Rβ2, and receptor activation requires binding of hIL-12 to both hIL-12Rβ1 and hIL-12Rβ2. The binding of hIL-12 to the receptor complex activates the Janus tyrosine kinases, Tyk2 and Jak2, associated with hIL-12Rβ1 and hIL-12Rβ2, respectively, to phosphorylate the cytoplasmic tails of the receptors. This results in the recruitment of signal transducer and activator of transcription 4 (STAT4). Homodimerization of STAT4 results in its release from the receptor and translocation of the phosphorylated STAT4 homodimer into the nucleus, where it binds to STAT4-binding elements of the IFN-γ gene to produce IFN-γ.

In some embodiments, the modified hIL-12p40 polypeptides described herein have modulated binding affinity for the hIL-12 receptor, in particular, modulated binding affinity for hIL-12Rβ1. In some embodiments, the modified hIL-12p40 polypeptides or hIL-12 muteins comprising the modified hIL-12p40 polypeptides described herein are partial agonists of the hIL-12 receptor. In some embodiments, upon association with hIL-12p35, the modified hIL-12p40 polypeptides described herein have decreased binding affinity for hIL-12Rβ1, as compared to the binding affinity of a wildtype or parent hIL-12p40 polypeptide.

In some embodiments, the modified hIL-12p40 polypeptide has binding affinity for hIL-12Rβ1 reduced by about 10%, alternatively by about 20%, alternatively by about 30%, alternatively by about 40%, alternatively by about 50%, alternatively by about 60%, alternatively by about 70%, alternatively by about 80%, alternatively by about 90%, alternatively by about 100% compared to binding affinity of a reference polypeptide lacking the two or more amino acid substitution, as determined by surface plasmon resonance (SPR). In some embodiments, the reduced binding affinity of the modified hIL-12p40 polypeptide to hIL-12Rβ1 results in a reduction in STAT4-mediated signaling compared to a reference polypeptide lacking the two or more amino acid substitutions. In some embodiments, the reduced binding affinity of the recombinant polypeptide to hIL-12Rβ1 results in a reduction in STAT3-mediated signaling compared to a reference polypeptide lacking the two or more amino acid substitution. In some embodiments, the STAT3 signaling and/or STAT4 signaling is determined by an assay selected from the group consisting of by a gene expression assay, a phospho-flow signaling assay, and an enzyme-linked immunosorbent assay (ELISA).

In some embodiments, the modified hIL-12p40 polypeptides described herein are partial agonists of STAT3-mediated signaling (“STAT3 signaling”) and/or STAT4 mediated signaling (“STAT4 signaling”). In some embodiments, the modified hIL-12p40 polypeptides described herein, upon association with hIL-12p35 activate STAT3 signaling and/or STAT4 signaling in some cell types, and result in decreased STAT3 and/or STAT3 signaling in other cell types as compared to the binding affinity of a wildtype or parent IL-12p40 polypeptide.

In some embodiments, the modified hIL-12p40 polypeptides described herein, upon association with hIL-12p35 activate STAT4 signaling in CD8+ T cells, and result in decreased STAT4 signaling, for example, at least about a 10%, 20%, 30%, 40%, 50%, 60%, or 70% decrease, in NK cells, as compared to a wildtype or parent hIL-12p40 polypeptide. As used throughout, an increase in signaling can be an increase of about 10%, 20%, 30%, 40%, 50%, 60%, 70%, 80%, 90%, 100%, 200%, 300%, 4005 or greater.

In some embodiments, the modified hIL-12p40 polypeptide, upon association with hIL-12p35, forms a dimer that activates interferon gamma (IFNγ) in CD8+ T cells and has decreased IFNγ signaling in NKT cells, for example, at least about a 10%, 20%, 30%, 40%, 50%, 60%, or 70% decrease, compared to a wildtype or parent hIL-12p40 polypeptide lacking the two or more amino acid substitutions.

Association with Carrier Molecules to Increase Duration of Action

The modified hIL-12p40 polypeptides described herein, the hIL-12 muteins comprising the modified hIL-12p40 polypeptides, or the hIL-13 muteins comprising the modified hIL-12p40 polypeptides can be modified to provide for an extended lifetime in vivo and/or extended duration of action in a subject. In the hIL-12 muteins, the hIL-12 p40 and/or the hll-12 p35 polypeptide can be modified to provide an extended lifetime and/or extended duration in a subject. In the hIL-23 muteins, the hIL-12 p40 and/or the human p19 polypeptide can be modified to provide an extended lifetime and/or extended duration in a subject. In some embodiments, the binding molecule can be conjugated to a carrier molecule(s) to provide desired pharmacological properties such as an extended half-life. In some embodiments, the binding molecule can be covalently linked to the Fc domain of IgG, albumin, or other molecules to extend its half-life, e.g., by pegylation, glycosylation, and the like, as known in the art. In some embodiments, the modified hIL-12p40 polypeptide, hIL-12 mutein, or hIL-23 muteinis modified to provide an extended duration of action in a mammalian subject and has a half-life in a mammalian subject of greater than 4 hours, alternatively greater than 5 hours, alternatively greater than 6 hours, alternatively greater than 7 hours, alternatively greater than 8 hours, alternatively greater than 9 hours, alternatively greater than 10 hours, alternatively greater than 12 hours, alternatively greater than 18 hours, alternatively greater than 24 hours, alternatively greater than 2 days, alternatively greater than 3 days, alternatively greater than 4 days, alternatively greater than 5 days, alternatively greater than 6 days, alternatively greater than 7 days, alternatively greater than 10 days, alternatively greater than 14 days, alternatively greater than 21 days, or alternatively greater than 30 days.

Modifications of the modified hIL-12p40 polypeptide, hIL-12 mutein or hIL-23 mutein to provide an extended duration of action in a mammalian subject include (but are not limited to) “:
conjugation of the modified hIL-12p40 polypeptide, hIL-12 mutein, or hIL-23 mutein to one or more carrier molecules;
conjugation of the modified hIL-12p40 polypeptide, hIL-12 mutein, or hIL-23 mutein to protein carrier molecules, optionally in the form of a fusion protein with additional polypeptide sequences (e.g, modified hIL-12 polypeptide-Fc fusions, hIL-12 mutein-Fc fusions, or hIL-23 mutein-Fc-fusions) and
conjugation to polymers, (e.g. water soluble polymers to provide a PEGylated hIL-12p40 polypeptide, hIL-12 mutein or hIL-23 mutein).

It is understood that more than one type of modification that provides for an extended duration of action in a mammalian subject may be employed with respect to a given modified hIL-12p40 polypeptide, hIL-12 mutein, or hIL-23 mutein. For example, a modified hIL-12p40 polypeptide, hIL-12 mutein, or hIL-23 mutein of the present disclosure may comprise both amino acid substitutions that provide for an extended duration of action as well as conjugation to a carrier molecule such as a polyethylene glycol (PEG) molecule.

### Protein Carrier Molecules

Examples of protein carrier molecules that can be covalently attached to the modified hIL-12p40 polypeptide, hIL-12 mutein, or hIL-23 mutein to provide an extended duration of action in vivo include, but are not limited to, albumins, antibodies, and antibody fragments, such as, Fc domains of IgG molecules.

### Fc Fusions

In some embodiments, the modified hIL-12p40 polypeptide, hIL-12 mutein, or hIL-23 mutein is conjugated to a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The binding molecule described herein can be conjugated to the entire Fc region, or a smaller portion that retains the ability to extend the circulating half-life of a chimeric polypeptide of which it is a part. In addition, full-length or fragmented Fc regions can be variants of the wild-type molecule. In a typical presentation, each monomer of the dimeric Fc can carry a heterologous polypeptide, the heterologous polypeptides being the same or different.

### Linkage of IL12 p35 and P40 Fc Subunits of A Dimeric Fc Domain

In some embodiments, the individual subunits of the IL 12 mutein are presented on a Fc scaffold. Zhao et al. “A new approach to produce IgG4-like bispecific antibodies,” Scientific Reports 11:18630 (2021); and Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8:38. doi: 10.3389/fimmu.2017.00038 (2017).

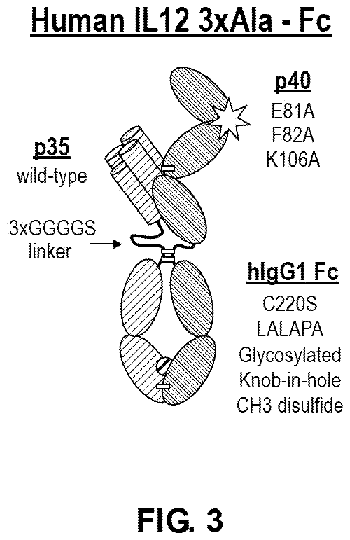

In some embodiments, any of the mutant hIL-12p40 polypeptides described herein can be in an IL-12 construct having extended half-life wherein the p35 and p40 subunits are individually attached to a Fc domain of an Fc heterodimer in a “knob-into-hole” format. See, for example, Ridgway, et al (1996) Protein Engineering 9 (7): 617-921. An exemplary hIL-12-Fc molecule is shown in FIG. 3, where a mutant hIL-12p40 comprising a E81A mutation, a F82A mutation, and a K106A mutation and a wildtype hIL-12-p35 are each attached via a linker to an hIgG1 Fc domain in a knob-into-hole format. This format can be used to improve manufacturing of hIL-12 muteins, achieve higher levels of expression and improve the homogeneity of the resulting manufactured product. See, e.g. Gillies, et al. U.S. Pat. No. 7,576,193 issued Aug. 18, 2009; Epstein, et al. Chinese Patent Application Serial No CN201410597561.4A published May 4, 2016; and Kim, et al., U.S. Pat. No. 11,087,249 issued Aug. 3, 2021; Zhao et al. “A new approach to produce IgG4-like bispecific antibodies,” Scientific Reports 11:18630 (2021); and Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8:38. doi: 10.3389/fimmu.2017.00038 (2017).

### Modifications of Fc Subunits to Promote Heterodimerization

In some embodiments, the monomers of the dimeric Fc domain may be modified so as to promote heterodimerization. A variety of techniques are established for the promotion of heterodimerization of Fc domains. See, e.g. Gillies, et al. United States Patent No. Kim, et al., U.S. Pat. No. 11,087,249, issued Aug. 3, 2021.

One example of a modification to the Fc domain monomers to promoter heterodimerization is the employment of the “knob-into-hole” modification.” See, for example, Ridgway, et al (1996) Protein Engineering 9 (7): 617-921; Atwell, et al (1997) J. Mol. Biol. 270:26-35; Carter, et al. U.S. Pat. No. 5,807,706 issued Sep. 15, 1998; Carter, et al U.S. Pat. No. 7,695,936 issued Apr. 13, 2010; Zhao et al. “A new approach to produce IgG4-like bispecific antibodies,” Scientific Reports 11:18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8:38. doi: 10.3389/fimmu.2017.00038 (2017). In some embodiments, any of the mutant hIL-12p40 polypeptides described herein can be in an hIL-12 construct having extended half-life wherein the p35 and p40 subunits are individually attached to a Fc domain of an Fc heterodimer. In some embodiments, the Fc domain comprises two Fc monomers wherein the CH3 domain of a first Fc monomer wherein the threonine at (EU numbering) position 366 is modified with a bulky residue (e.g. a T366W) create a “knob” and the substitution, and a second Fc monomer comprising one or more substitutions in complementary residues of the CH3 domain of the second Fc monomer to create a pocket or “hole” to receive the bulky residue, for example by amino acid substitutions such as T366S, L368A, and/or Y407V.

### Modifications to Reduce Effector Functions of Fc

In some embodiments, the Fc domain may be modified to reduce effector function. Modification of Fc domains to reduce effector function are well known in the art. See, e.g., Wang, et al. (2018) IgG Fc engineering to modulate antibody effector functions, Protein Cell 9 (1): 63-73. For example, mutation of the lysine residue at position 235 (EU numbering) from lysine (L) to glutamic acid (E) is known to reduce effector function by reducing FcgR and Clq binding. Alegre, et al. (1992) J. Immunology 148:3461-3468. Additionally, substitution of the two lysine (L) residues at positions 234 and 235 (EU numbering) in the IgG1 hinge region with alanine (A), i.e., L234A and L235A, results in decreased complement dependent cytotoxicity (CDC) and antibody dependent cellular cytotoxicity (ADCC). Hezereh, et al., (2001) J. Virol 75 (24): 12161-68. Furthermore, mutation of the proline at position 329 (EU numbering) to alanine (P329A) or glycine, (P329G) mitigates effector function and may be combined with the L234A and L235A substitutions. In some embodiments, the Fc domains of the compositions of the present invention may comprises the amino acid substitutions L234A/L235A/P329A (“EU numbering) or L234A/L235A/P329G (EU numbering).

In some embodiments, the incorporation of the hinge region contains an unpaired cysteine residue at position 220 (EU numbering) that typically, in a complete immunoglobulin molecule, binds to a cysteine on a light chain. When only the Fc domain is used comprising the hinge domain, the unpaired cysteine in the hinge domain creates the potential of the formation of improper disulfide bonds. Consequently, in some embodiments the cysteine at position 220 (C220, numbered in accordance with EU numbering) is substituted with an amino acid that does not promote disulfide bonding. In some embodiments, the Fc domain comprises a C220S mutation.

In some embodiments, the Fc domain is modified to eliminate N-linked or O-linked glycosylation sites. A glycosylated variants of Fc domains, particularly of the IgG1 subclass are known to be poor mediators of effector function. Jefferis, et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc domains of the compositions of the present disclosure comprise one or modifications to eliminate N- or O linked glycosylation sites. Examples of modifications at N297 to eliminate glycosylation sites in the Fc domain include the amino acid substitutions N297Q and N297G.

Substitutions that Increase Resistance to Proteolytic Cleavage

In some embodiments, the human IL-12p40 (hIL-12p40) comprises an amino acid substitution of the lysine (K) residue at position 260 (K260) of the mature form of the human p40 polypeptide (SEQ ID NO: 148, corresponding to position 282 of the human p40 precursor polypeptide SEQ ID NO: 1). As described in Webster, et al (U.S. Pat. No. 7,872,107 issued Jan. 18, 2011), a substitution at position 260 of the mature human p40 polypeptide renders the human p40 polypeptide resistant to proteolytic cleavage. In some embodiments, human IL-12p40 (hIL-12p40) comprises a substitution of the lysine at position 260 of the mature p40 polypeptide with a non-basic amino acid. In some embodiments the non-basic amino acid is selected from the group consisting of alanine, glycine, asparagine or glutamine. In some embodiments, human IL-12p40 (hIL-12p40) comprises a mutation at position 260 selected from the group consisting of K260G, K260A, K260N, K260Q (numbered in accordance with the mature form of hp40) or K282G, K282A, K282N, K282Q (numbered in accordance with SEQ ID NO: 1, the precursor form of hp40).

### IL12 Partial Agonists

In some embodiments, the present disclosure provides heterodimeric hIL 12Fc muteins comprising modified hIL-12p40 polypeptides which have improved pharmacological or therapeutic properties, and methods of using such compositions.

In some embodiments, the present disclosure provides a composition comprising a first polypeptide of the formula #1

$$P 35 - L {1}_{a} - {H}_{b} - {Fc}_{1} \qquad [ 1 ]$$

wherein:
L1 is a linker from 1-30 amino acids, optionally a GSA linker
a and b are independently selected from 0 (absent) or 1 (present),
H is a hinge region of a human immunoglobulin independently selected from the group consisting of the IgG1, IgG2, IgG3 and IgG4 upper hinge, optionally comprising the amino acid substitution C220S (EU numbering);
Fc₁ is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc₂, and
P35 is a human p35 polypeptide having at least 90%, alternatively at least 91%, alternatively at least 92%, alternatively at least 93%, alternatively at least 94%, alternatively at least 95%, alternatively at least 96%, alternatively at least 97%, alternatively at least 98%, alternatively at least 99% sequence identity, or 100% sequence identity to SEQ ID NO: 1);
   and a second polypeptide of the formula #2:

$$P {40}^{m} - L {2}_{c} - {H}_{d} - {Fc}_{2} \qquad [ 2 ]$$

wherein
L2 is a linker from 1-30 amino acids, optionally a GSA linker,
c and d are independently selected from 0 (absent) or 1 (present),
H is a hinge region of a human immunoglobulin independently selected from the group consisting of the IgG1, IgG2, IgG3 and IgG4 upper hinge, optionally comprising the amino acid substitution C220S (EU numbering);
Fc₂ is a polypeptide comprising the lower hinge, CH2 and CH3 domains of a human immunoglobulin selected from the group consisting of IgG1, IgG2, IgG3 and IgG4, comprising one or more amino acid substitutions promote heterodimerization with Fc₁, and
P40^{m} is a modified human IL-12p40 polypeptide described herein, for example, a modified hIL-12p40 polypeptide sequence from Table 7 (i.e., selected from the group consisting of SEQ ID NO:7-SEQ ID NO: 46 and SEQ ID NO: 151-190). Optionally, the modified human IL-12p40 polypeptide comprising one or more amino acid substitutions at positions selected from the group consisting of positions W37, P39, D40, A41, K80, E81, F82, K106, E108, D115, H216, K217, L218, and K219 numbered in accordance with SEQ ID NO: 1. Optionally, the the one or more amino acid substitutions reduce the binding affinity of P40m for the extracellular domain (ECD) of hIL12Rb1 by at least 5%, optionally by at least 10%, optionally by at least 20%, optionally by at least 30%, optionally by at least 40%, optionally by at least 50%, optionally by at least 60%, optionally by at least 70%, relative to the binding affinity of wild type hP40 for the extracellular domain (ECD) of hIL 12Rb1 as determined by surface plasmon resonance,
   wherein the polypeptide of formula 1 and the polypeptide of formula 2 are linked by at least one interchain disulfide bond.

The heterodimeric hIL-12Fc muteins of the present disclosure comprise a modified hIL-12p40 polypeptide comprising one or more amino acid substitutions, modifications and/or deletions at the interface with the extracellular domain of IL 12Rβ1 which result in a reduction of the binding affinity of the modified hIL-12p40 polypeptide to IL 12Rβ1 relative to the mature form of wildtype IL-12p40 (SEQ ID NO:1). In some embodiments, the binding affinity of the modified hIL-12p40 polypeptide for the extracellular domain of IL 12Rβ1 is reduced by about 10% to about 100% compared to binding affinity of a reference polypeptide (wildtype hIL-12p40) as determined by surface plasmon resonance (SPR) spectroscopy. In some embodiments, the modified hIL-12p40 polypeptide has at least 70% sequence identity to SEQ ID NO: 1 (e.g., at least 70%, 75%, 80%, 85%, 90%, 91%, 92%, 93%, 94%, 95%, 96%, 97%, 98%, or 99% sequence identity to SEQ ID NO:1). In some embodiments, the modified hIL-12p40 polypeptide comprises one or more amino acid substitutions at residues selected from the group consisting of W37, P39, D40, A41, K80, E81, F82, K106, E108, D115, H216, K217, L218, and K219 numbered in accordance with SEQ ID NO: 1. In some embodiments, the modified hIL-12p40 polypeptide comprises one or more amino acid substitutions at residues selected from the group consisting of E81, F82, K106, and K217 numbered in accordance with SEQ ID NO: 1. In some embodiments the one or more amino acid substitutions at positions W37, P39, D40, A41, K80, E81, F82, K106, E108, D115, H216, K217, L218, and K219 are selected from the group consisting of P39A, D40A, E81A, F82A, K106A, D109A, K217A, K219A. In some embodiments, the modified hIL-12p40 polypeptide comprises two or more amino acid substitutions at residues selected from the group consisting of W37, P39, D40, A41, K80, E81, F82, K106, E108, D115, H216, K217, L218, and K219 numbered in accordance with SEQ ID NO: 3. In some embodiments, the modified hIL-12p40 polypeptide comprises two or more amino acid substitutions at residues selected from the group consisting of E81, F82, K106, and K217 numbered in accordance with SEQ ID NO: 1. In some embodiments, the modified hIL-12p40 polypeptide comprises three or more amino acid substitutions at residues selected from the group consisting of W37, P39, D40, A41, K80, E81, F82, K106, E108, D115, H216, K217, L218, and K219 numbered in accordance with SEQ ID NO:3. In some embodiments wherein the modified hIL-12p40 polypeptide comprises two or more amino acid substitutions at W37, P39, D40, A41, K80, E81, F82, K106, E108, D115, H216, K217, L218, and K219, the two or more substitutions comprise a set of amino acid substitutions selected from the group consisting of the sets of amino acid substitutions: E81A/F82A, E81K/F82A, E81L/F82A, E81H/F82A and E81S/F82A. In some embodiments, the modified hIL-12p40 polypeptide comprises three or more amino acid substitutions at residues selected from the group consisting of E81, F82, K106, and K217 numbered in accordance with SEQ ID NO:3. In some embodiments, the modified hIL-12p40 polypeptide comprises three or more amino acid substitutions at residues selected from the group consisting of W37, P39, D40, A41, K80, E81, F82, K106, E108, D115, H216, K217, L218, and K219 numbered in accordance with SEQ ID NO: 1. In some embodiments wherein the modified hIL-12p40 polypeptide comprises three or more amino acid substitutions at W37, P39, D40, A41, K80, E81, F82, K106, E108, D115, H216, K217, L218, and K219, the three or more substitutions comprise a set of amino acid substitutions selected from the group consisting of the sets of amino acid substitutions: W37A/E81A/F82A; E81A/F82A/K106A; E81A/F82A/K106A/K219A, E81A/F82A/K106N, E81A/F82A/K106Q, E81A/F82A/K106T, and E81A/F82A/K106R. In some embodiments, the modified hIL-12p40 polypeptide comprises four or more amino acid substitutions at residues selected from the group consisting of W37, P39, D40, A41, K80, E81, F82, K106, E108, D115, H216, K217, L218, and K219 numbered in accordance with SEQ ID NO: 1. In some embodiments wherein the modified hIL-12p40 polypeptide comprises four or more amino acid substitutions at W37, P39, D40, A41, K80, E81, F82, K106, E108, D115, H216, K217, L218, and K219, the four or more substitutions comprise a set of amino acid substitutions selected from the group consisting of the sets of amino acid substitutions: E81A/F82A/K106A/K217A, 81A/F82A/K106A/E108A/D115A and P39A/D40A/E81A/F82A.

In some embodiments, the modified hIL-12p40 polypeptide does not comprise the set of amino acid substitutions E81A/F82A. In some embodiments, the modified hIL-12p40 polypeptide does not comprise the set of amino acid substitutions E81A/F82A/K106A. In some embodiments, the modified hIL-12p40 polypeptide does not comprise the set of amino acid substitutions E81A/F82A/K106A/K217A.

Properties of the Heterodimeric hIL-12Fc Muteins

In some embodiments, the heterodimeric hIL 12Fc muteins comprising the modified hIL-12p40 polypeptides described herein provide cell-type biased signaling of the downstream signal transduction mediated through the IL12 receptor compared to a reference polypeptide (e.g., wild type hIL-12). In some embodiments, the reduced binding affinity of the modified hIL-12p40 polypeptide of the heterodimeric hIL-12Fc mutein to IL12Rβ1 results in a reduction in STAT4-mediated signaling compared to a reference polypeptide (wt hIL-12). In some embodiments, the heterodimeric hIL-12Fc muteins comprising the modified hIL-12p40 polypeptides described herein are partial agonists of STAT3-mediated signaling (“STAT3 signaling”) and/or STAT4 mediated signaling (“STAT4 signaling”). In some embodiments, the heterodimeric hIL-12Fc muteins have reduced STAT3-mediated signaling compared to a reference polypeptide (wt hIL-12). In some embodiments, the STAT3 signaling and/or STAT4 signaling is determined by an assay selected from the group consisting of by a gene expression assay, a phospho-flow signaling assay, and an enzyme-linked immunosorbent assay (ELISA).

The heterodimeric hIL-12Fc muteins comprising the modified hIL-12p40 polypeptides described herein provide selective activation of certain cell types which provides beneficial properties, such as anti-inflammatory properties, and/or have reduced undesirable properties, such as pro-inflammatory side effects compared to wt hIL-12. In some embodiments, the heterodimeric hIL-12Fc muteins comprising the modified hIL-12p40 polypeptides described herein provide cell-type biased signaling of the downstream signal transduction mediated through the IL-12 receptor compared to a reference polypeptide (e.g., wild type hIL-12). For example, the heterodimeric hIL-12Fc muteins of the present disclosure retain the property of wild-type hIL-12 to stimulate or activate IL-12 signaling in CD8+ T cells but exhibit a reduction of IFNγ and/or STAT4-mediated signaling in natural killer (NK) cells providing a reduction in the inflammatory response. In some embodiments, the cell-type biased signaling of the heterodimeric hIL-12Fc muteins comprising the modified hIL-12p40 polypeptides described herein of the present disclosure includes the ability to provide substantial IL12 signaling (e.g., at least 30%, alternatively at least 40%, alternatively at least 50%, alternatively at least 60%, alternatively at least 70%, alternatively at least 80%, alternatively at least 90%) of the activity of wt hIL 12 in CD8+ T cells. In some embodiments, the heterodimeric hIL-12Fc muteins described herein increase STAT4 signaling in CD8+ T cells by 10%, 20%, 30%, 40%, 50%, 60%, 70%, 80%, 90%, 100%, 200%, 300%, 400% or greater and decrease STAT4 signaling in NK cells, for example, at least about a 10%, 20%, 30%, 40%, 50%, 60%, or 70% decrease, as compared to a reference polypeptide (wt hIL-12). In some embodiments, the heterodimeric hIL-12Fc muteins described herein activate interferon gamma (IFNγ) in CD8+ T cells by 10%, 20%, 30%, 40%, 50%, 60%, 70%, 80%, 90%, 100%, 200%, 300%, 400% and decreased IFNγ signaling in NKT cells by at least 10%, 20%, 30%, 40%, 50%, 60%, or 70% compared to a reference polypeptide (wt hIL-12). Thus, the heterodimeric hIL12Fc muteins comprising the hP40 muteins described herein exhibit reduced activation of NK cells while retaining the ability to stimulate CD8+T cells.

### GSA Linkers:

In the polypeptides of formulae [1] and [2], Fc domain fusions incorporating a modified hIL-12p40 polypeptide and/or p35 may optionally contain a GSA linker molecule between the modified hIL-12p40 polypeptide and the upper hinge. As used herein the term “GSA linker” refers to a polypeptide having 4, 5, 6, 7, 8,9, 10, 11, 12, 13, 14, 15, 16, 17, 18, 19 or 20 amino acids comprised of amino acids selected from the group consisting of glycine, serine and alanine. In some embodiments, the polypeptide linker is a glycine-serine polymer of the structure (GGGGSm)n, (GGGSm)n, (GGGAm)n and (GGGGAm)n, and combinations thereof, where m, n, and o are each independently selected from 1, 2, 3 or 4. In the construction of such polymers, it may be desirable to avoid repeated “GSG” sequences which potentially may provide introduction of a non-naturally occurring glycosylation site. Exemplary glycine-serine linkers include but are not limited to the monomers: GGGS (referred to as “G4S”), GGGGA (referred to as “G4A”), GGGS (referred to as “G3S”) and GGGA (referred to as “G3A”), or homopolymers (e.g. “GGGGSGGGGS” also referred to as (G4S) 2) or heteropolymers thereof. Exemplary GSA linkers are provided in Table 2 below:

TABLE 2
Exemplary GSA Linkers

| | Amino Acid | |
|---|---|---|
| Name/Ref | Sequence | SEQ ID NO: |
| (G3A)2 | GGGAGGGA | 87. |
| (G3A)4 | GGGAGGGAGGGA | 88. |
| (G3AG2)2 | GGGAGGGGGAGG | 89. |
| (G3S)3 | GGGSGGGSGGGS | 90. |
| (G3SG2)2 | GGGSGGGGGSGG | 91. |
| (G4A)2 | GGGGAGGGGA | 92. |
| (G4a)3 | GGGGAGGGGAGGGGA | 93. |
| (G4AG)2 | GGGGAGGGGGAG | 94. |
| (G4AG2)2 | GGGGAGGGGGGAGG | 95. |
| (G4S)2 | GGGGSGGGGS | 96. |
| (G4S)3 | GGGGSGGGGSGGGGS | 97. |
| (G4SG)2 | GGGGSGGGGGSG | 98. |
| (G4SG2)2 | GGGGSGGGGGGSGG | 99. |
| (G5AG)2 | GGGGGAGGGGGGAG | 100. |
| (G5SG)2 | GGGGGSGGGGGGSG | 101. |
| G2AG | GGAG | 102. |
| G3A | GGGA | 103. |
| G3A-G3S | GGGAGGGS | 104. |
| G3A-G3S- G3A | GGGAGGGSGGGA | 105. |
| G3A-G4S | GGGAGGGGS | 106. |
| G3A-G4SA | GGGAGGGGA | 107. |
| G3AG2 | GGGAGG | 108. |
| G3AG2 G4AG | GGGAGGGGGGAG | 109. |
| G3S | GGGS | 110. |
| G3S-G3A-GGGS | GGGSGGGAGGGS | 111. |
| G3SG2-G4SG | GGGSGGGGGGSG | 112. |
| G4A | GGGGA | 113. |
| G4A-G4S-G4A | GGGGAGGGGSGGGGA | 114. |
| G4A-G4S-G4S | GGGGAGGGGSGGGGS | 115. |
| G4A-G4AG | GGGGAGGGGAG | 116. |
| G4A-G4S | GGGGAGGGGS | 117. |
| G4A-G4SG | GGGGAGGGGSG | 118. |
| G4AG | GGGGAG | 119. |
| G4AG G4SG2 | GGGGAGGGGGSG | 120. |
| G4AG-G4AG | GGGGAGGGGGAG | 121. |
| G4AG-G4SG | GGGGAGGGGGSG | 122. |
| G4AG2 | GGGGAGG | 123. |
| G4AG2 G3SG2 | GGGGAGGGGGSGG | 124. |
| G4S | GGGGS | 125. |
| G4S- G4A- G4A | GGGGSGGGGAGGGGA | 126. |
| G4S- G4A- G4S | GGGGSGGGGAGGGGS | 127. |
| G4S- G4S- G4A | GGGGGGGGSGGGGA | 128. |
| G4S-G4A | GGGGSGGGGA | 129. |
| G4S-G4AG | GGGGSGGGGAG | 130. |
| G4S-G4SG | GGGGSGGGGSG | 131. |
| G4SG G3AG2 | GGGGSGGGGAGG | 132. |
| G4SG-G4AG | GGGGSGGGGGAG | 133. |
| G4SG-G4SG | GGGGSGGGGGSG | 134. |
| G4SG2 G5SG | GGGGSGGGGGGSG | 135. |
| G5AG | GGGGGAG | 136. |
| G5SG G4AG2 | GGGGGSGGGGGAGG | 137. |
| GAG2 | GAGG | 138. |
| GSG2 | GAGG | 139. |

### Fc1 and Fc2:

The hIL-12 partial agonists of the present disclosure are heterodimers comprising polypeptides of the formulae [1] and [2], which each incorporate an Fc region (Fc1 and Fc2) of a human immunoglobulin molecule modified to promote heterodimerization. As used herein the term “Fc” and “Fc monomer” are used interchangeably herein to designate the monomeric polypeptide subunit of an Fc dimer. An Fc comprises an amino acid sequence (from amino to carboxy terminal) comprising a lower hinge domain and the CH2 and CH3 domains of a human immunoglobulin molecule. In some embodiments, the Fc monomer is a polypeptide comprising the lower hinge domain and the CH2 and CH3 domains of a human immunoglobulin molecule domains of human IgG1, human IgG2, human IgG3 and human IgG4 hinge domains. The CH2 domain of hIgG1 corresponds to amino acid residues 238-337 (EU numbering) and is provided as SEQ ID NO: 142. The CH2 domain of hIgG1 corresponds to amino acid residues 346-442 (EU numbering) and is provided as SEQ ID NO: 147.

The polypeptides of the formulae [1] and [2] each incorporate a lower hinge region of a human immunoglobulin. As used herein, the term “lower hinge” or “LH” refers to an amino acid sequence corresponding to amino acid residues 221-229 (EU numbering) of a human immunoglobulin molecule. In some embodiments, the lower hinge region is a naturally occurring lower hinge region of a human immunoglobulin selected from the LH regions of IgG1, IgG2, IgG3 and IgG4 lower hinge domains. In some embodiments, the lower hinge region is the lower hinge region of a human IgG1 immunoglobulin. In some embodiments, the lower hinge region is the lower hinge region of a human IgG1 immunoglobulin comprising the nonameric amino acid sequence: DKTHTCPPC (SEQ ID NO: 140).

In some embodiments, Fc1 and Fc2 are derived from a polypeptide corresponding to amino acids 221-447 (EU numbering) of the human IgG1 immunoglobulin having the amino acid sequence (EU numbering indicated, SEQ ID NO: 141):

```
                230 240 250 260
DKTHTCPPCP APELLGGPSV FLFPPKPKDT LMISRTPEVT
                270 280. 290. 300
CVVVDVSHED PEVKFNWYVD GVEVHNAKTK PREEQYNSTY
                310 320 330 340
RVVSVLTVLH QDWLNGKEYK CKVSNKALPA PIEKTISKAK
                350 360 370 380
GQPREPQVYT LPPSRDELTK NQVSLTCLVK GFYPSDIAVE
                390 400 410 420
WESNGQPENN YKTTPPVLDS DGSFFLYSKL TVDKSRWQQG
                430 440 447
NVFSCSVMHE ALHNHYTQKS LSLSPGK
```

As indicated in above sequence, the C-terminal residue of the wild-type form of the IgG1 Fc domain is a lysine, referred to as K447 in accordance with EU numbering. The K447 is inconsistently removed by the producer cell during recombinant product. As a result, the population of recombinant Fc monomers may be heterogenous in that some fraction of the recombinantly produced Fc monomers will contain K447 and others will not. Such inconsistent proteolytic processing by producer cells may therefore result in a heterogenous population of hIL12Fc. Typically, particularly in the context of human pharmaceutical agents, such heterogeneity of the active pharmaceutical ingredient is to be avoided. Consequently, in addition to modifications to the Fc monomer sequence promote heterodimerization, the present disclosure provides Fc monomers that further comprising a deletion of the C-terminal K447 or a deletion of G446 and K447 and nucleic acid sequences encoding Fc monomers comprising a: (a) a deletion of the lysine residue at position 447 (K447,EU numbering, abbreviated as AK447 or des-K447), or (b) deletion of both the glycine at position 456 (G446 EU numbering, abbreviated as des-G446) and K447 (this double deletion of G446 and K447 being referred to herein as des-G446/des-K447 or AG446/AK447).

### Modifications of Fc Subunits to Promote Heterodimerization

As provided in formulae [1] and [2] above, the Fc1 and Fc2 monomers of the dimeric Fc contain amino acid substitutions that promote heterodimerization between Fc1 and Fc2. A variety of techniques are established for the promotion of heterodimerization of Fc domains. See, e.g. Gillies, et al. United States Patent No. Kim, et al., U.S. Pat. No. 11,087,249, issued Aug. 3, 2021. In some embodiments, the modifications to promoter heterodimerization are the HF-TA mutations and the HA-TF mutations as described in Moore, et al (2011) mAbs 3 (6): 546-557. The HF-TA method employs the S364H/T394F substitutions on one Fc monomer and the Y349T/F405A substitutions on the complementary Fc monomer. The (HA-TF) method employs the S364H/F405A substitutions on one Fc monomer and the Y349T/T394F substitutions on the complementary Fc monomer. Alternatively the ZW1 heterodimerization method employs the T350V/L351Y/F405A/Y407V substitutions on one Fc monomer and the T350V/T366L/K392L/T394W substitutions on the complementary Fc monomer. Von Kreudenstein, et al (2013) mAbs, 5 (5): 646-654. Also the EW-RVT heterodimerization method employs the K360E/K409W substitutions on one Fc monomer and the Q347R/D399V/F405T substitutions on the complementary Fc monomer. Choi, et al (2015) Molecular Immunology 65 (2): 377-83.

In one embodiment, the Fc1 and Fc2 are modified to promote heterodimerization is the employment of the “knob-into-hole” (abbreviated KiH) modification as exemplified herein. The KiH modification comprises one or more amino acid substitutions in a first Fc monomer (e.g. Fc1) that create a bulky “knob” domain on a first Fc and one or more amino acid substitutions on a second Fc monomer (e.g. Fc2) that create a complementary pocket or “hole” to receive the “knob” of the first Fc monomer.

A variety of amino acid substitutions have been established for the creation of complementary knob and hole Fc monomers. See, e.g. Ridgway, et al (1996) Protein Engineering 9 (7): 617-921; Atwell, et al (1997) J. Mol. Biol. 270:26-35; Carter, et al. U.S. Pat. No. 5,807,706 issued Sep. 15, 1998; Carter, et al U.S. Pat. No. 7,695,936 issued Apr. 13, 2010; Zhao et al. “A new approach to produce IgG4-like bispecific antibodies,” Scientific Reports 11:18630 (2021); Cao et al. “Characterization and Monitoring of a Novel Light-heavy-light Chain Mispair in a Therapeutic Bispecific Antibody,” and Liu et al. “Fc Engineering for Developing Therapeutic Bispecific Antibodies and Novel Scaffolds”. Frontiers in Immunology. 8:38. doi: 10.3389/fimmu.2017.00038 (2017).

In some embodiments, the Fc domain comprises two Fc monomers wherein the CH3 domain of a first Fc monomer wherein the threonine at (EU numbering) position 366 is modified with a bulky residue (e.g. a T366W) create a “knob” and the substitution, and a second Fc monomer comprising one or more substitutions in complementary residues of the CH3 domain of the second Fc monomer to create a pocket or “hole” to receive the bulky residue, for example by amino acid substitutions such as T366S, L368A, and/or Y407V.

In one embodiment, the Fc1 monomer of formula 1 is a “knob” modified Fc monomer comprising the amino acid substitution T366W and the Fc2 monomer of formula 2 is a “hole” modified Fc comprising the set of amino acid substitutions T366S/L368A/Y407V.

Alternatively, the Fc1 monomer of formula 1 is a “hole” modified Fc monomer comprising the set of amino acid substitutions T366S/L368A/Y407V and the Fc2 monomer of formula 2 is a “knob” modified Fc monomer comprising the amino acid substitution T366W.

An example of an engineered Fc heterodimeric pair comprising complementary KiH modifications is provided in Table 3 below:

TABLE 3
Amino Acid Substitution Sets of Complementary
IgG1 KiH Heterodimeric Pairs

| Fc | | |
|---|---|---|
| Dimer | Fc | Amino Acid Substitution Set |
| No. | Monomer | (EU Numbering) |
| 1 | Knob | T366W |
| | Hole | T366S/L368A/Y407V |

As noted, the heterodimeric hIL-12Fc muteins of the present disclosure are provided as a complementary heterodimeric pair of polypeptides of the formulae [1] and [2] wherein the first and second polypeptide are linked by at least one disulfide bond. In some embodiments, the incorporation of a disulfide bond between the polypeptides of formulae [1] and [2] may be achieved by cysteine substitutions at particular points within the Fc1 and Fc2 domains. In one embodiment, the Fc1 domain of the polypeptide of formula [1] is derived from the Fc domain of hIgG1 comprising an amino acid substitution S354C (EU numbering) and the Fc2 domain of the polypeptide of formula [2] is derived from the Fc domain of hIgG1 comprising an amino acid substitution Y349C (EU numbering) to provide a disulfide bond between the S354C of Fc1 and Y349C of Fc2. Alternatively, the Fc1 domain of the polypeptide of formula [1] is derived from the Fc domain of hIgG1 comprising an amino acid substitution Y349C (EU numbering) and the Fc2 domain of the polypeptide of formula [2] is derived from the Fc domain of hIgG1 comprising an amino acid substitution S354C (EU numbering) to provide a disulfide bond between the S354C of Fc1 and Y349C of Fc2.

Further examples of complementary KiH engineered heterodimeric Fc pairs that may be used in the practice of the present disclosure are provided in Table 4 below.

TABLE 4
Knob-into-Hole Fc Dimer Pairs

| Fc | | | | | Fc Amino Acid | |
|---|---|---|---|---|---|---|
| Dimer | Monomer | Monomer | UH | UH | Substitutions | Fc |
| Pair. | Type | SEQ ID | Sequence | SEQ ID | (EU Numbering) | Seq ID |
| 2 | Knob | 19 | wt | 143 | T366W | 145 |
| | Hole | 20 | wt | 143 | T366S/L368A/Y407V | 146 |
| 3 | Knob | 21 | C220S | 144 | T366W | 145 |
| | Hole | 22 | C220S | 144 | T366S/L368A/Y407V | 146 |
| 4 | Knob | 23 | wt | 143 | T366W | 145 |
| | Hole | 24 | wt | 143 | T366S/L368A/Y407V | 146 |
| 5 | Knob | 25 | C220S | 144 | S354C/T366W | 145 |
| | Hole | 26 | C220S | 144 | Y349C/T366S/L368A/ | 146 |
| | | | | | Y407V | |

### Additional Fc Modifications

In addition to the modifications to promote heterodimerization of the Fc1 and Fc2 domains, Fc1 and Fc2 may optionally provide additional amino acid modifications that mitigate effector function, eliminate one or more glycosylation sites.

### Modifications to Reduce Effector Functions

In some embodiments, the Fc domain may be modified to substantially reduce binding to Fc receptors (FcγR and FcR) which reduces or abolishes antibody directed cytotoxicity (ADCC) effector function. Modification of Fc domains to reduce effector function are well known in the art. See, e.g. Wang, et al. (2018) IgG Fc engineering to modulate antibody effector functions, Protein Cell 9(1): 63-73. For example, mutation of the lysine residue at position 235 (EU numbering) from lysine (L) to glutamic acid (E) is known to reduce effector function by reducing FcgR and C1q binding. Alegre, et al. (1992) J. Immunology 148:3461-3468. Additionally, substitution of the two lysine (L) residues at positions 234 and 235 (EU numbering) in the IgG1 hinge region with alanine (A), i.e. L234A and L235A, results in decreased complement dependent cytotoxicity (CDC) and antibody dependent cellular cytotoxicity (ADCC). Hezereh et al., (2001) J. Virol 75 (24): 12161-68. Furthermore, mutation of the proline at position 329 (EU numbering) to alanine (P329A) or glycine, (P329G) mitigates effector function and may be combined with the L234A and L235A substitutions. In some embodiments, the Fc domains of the compositions of the present invention may comprises the amino acid substitutions L234A/L235A/P329A (EU numbering) referred to as the “LALAPA” substitutions or L234A/L235A/P329G (EU numbering) referred to as the “LALAPG” substitutions. Examples of paired KiH Fc dimeric constructs that may be incorporated into the hIL12 and heterodimeric hIL23Fc muteins of the present disclosure are provided in Table 5 below:

TABLE 5
Amino Acid Substitution Sets of Complementary IgG1 KiH UH/Fc Heterodimeric
Pairs Comprising Mutations to Reduce Effector Function

| Fc | | |
|---|---|---|
| Dimer | UH Fc | Amino Acid Substitution Set |
| No. | Monomer | (EU Numbering) |
| 6 | Knob | L234A/L235A/P329A/T366W/ΔK457 |
| | Hole | L234A/L235A/P329A/T366S/L368A/Y407V/ΔK457 |
| 7 | Knob | C220S/L234A/L235A/P329A/T366W/ΔK457 |
| | Hole | C220S/L234A/L235A/P329A/T366S/L368A/Y407V/ΔK457 |
| 8 | Knob | L234A/L235A/P329A/S354C/T366W/ΔK457 |
| | Hole | L234A/L235A/P329A/Y349C/T366S/L368A/Y407V/ΔK457 |
| 9 | Knob | C220S/L234A/L235A/P329A/S354C/T366W/ΔK457 |
| | Hole | C220S/L234A/L235A/P329A/Y349C/T366S/L368A/Y407V/ΔK457 |
| 10 | Knob | L234A/L235A/P329G/T366W/ΔK457 |
| | Hole | L234A/L235A/P329G/T366S/L368A/Y407V/ΔK457 |
| 11 | Knob | C220S/L234A/L235G/P329A/T366W/ΔK457 |
| | Hole | C220S/L234A/L235G/P329A/T366S/L368A/Y407V/ΔK457 |
| 12 | Knob | L234A/L235A/P329G/S354C/T366W/ΔK457 |
| | Hole | L234A/L235A/P329G/Y349C/T366S/L368A/Y407V/ΔK457 |
| 13 | Knob | C220S/L234A/L235A/P329G/S354C/T366W/ΔK457 |
| | Hole | C220S/L234A/L235A/P329G/Y349C/T366S/L368A/Y407V/ΔK457 |
| 14 | Knob | L234A/L235E/G237A/A330S/P331S/T366W/ΔK457 |
| | Hole | L234A/L235E/G237A/A330S/P331S/T366S/L368A/Y407V/ΔK457 |
| 15 | Knob | C220S L234A/L235E/G237A/A330S/P331S/T366W/ΔK457 |
| | Hole | C220S/L234A/L235E/G237A/A330S/P331S/T366S/L368A/Y407V/ΔK457 |
| 16 | Knob | L234A/L235E/G237A/A330S/P331S/S354C/T366W/ΔK457 |
| | Hole | L234A/L235E/G237A/A330S/P331S/Y349C/T366S/L368A/Y407V/ΔK457 |
| 17 | Knob | C220S/L234A/L235E/G237A/A330S/P331S/S354C/T366W/ΔK457 |
| | Hole | C220S/L234A/L235E/G237A/A330S/P331S/Y349C/T366S/L368A/Y407V/ΔK457 |
| 18 | Knob | L234F/L235E/P331S/T366W/ΔK457 |
| | Hole | L234F/L235E/P331S/T366S/L368A/Y407V/ΔK457 |
| 19 | Knob | C220S/L234F/L235E/P331S/T366W/ΔK457 |
| | Hole | C220S/L234F/L235E/P331S//L368A/Y407V/ΔK457 |
| 20 | Knob | L234F/L235E/P331S/S354C/T366W/ΔK457 |
| | Hole | L234F/L235E/P331S/Y349C/T366S/L368A/Y407V/ΔK457 |
| 21 | Knob | C220S/S/L234F/L235E/P331S/354C/T366W/ΔK457 |
| | Hole | C220S/L234F/L235E/P331S/Y349C/T366S/L368A/Y407V/ΔK457 |

### Sequence Modifications to Extend Duration of Action:

In some embodiments the amino acid sequence of the Fc1 and/or Fc2 monomers may incorporate amino acid substitutions which extend the duration of action of the molecule and prevent clearance. In some embodiments, such modifications to the Fc monomer include the amino acid substitutions M428L and M434S (EU numbering) referred to as the “LS” modification. The LS modification may optionally be combined with amino acid substitutions to reduce effector function and provide for disulfide bonds between Fc1 and Fc2. Table 11 below provides exemplary Fc1 and Fc1 heterodimeric pairs possessing complementary sequence modifications to promote heterodimerization that may be employed in the design of the Fc1 and Fc2 polypeptides of the formulae [1] and [2].

The following Table 6 provides exemplary Fc heterodimeric pairs which may be used in the preparation of Fc1 and Fc2 polypeptides of the heterodimeric hIL 12Fc muteins of the present disclosure:

TABLE 6
Amino Acid Substitution Sets of Complementary IgG1 KiH UH/Fc Heterodimeric Pairs
Comprising Mutations to Reduce Effector Function and LS Halflife Extensions

| Fc | | |
|---|---|---|
| Dimer | UH/Fc | Amino Acid Substitution Set |
| No. | Monomer | (EU Numbering) |
| 22 | Knob | L234A/L235A/P329A/T366W/M428L/M434S/ΔK457 |
| | Hole | L234A/L235A/P329A/T366S/L368A/Y407V/M428L/M434S/ΔK457 |
| 23 | Knob | C220S/L234A/L235A/P329A/T366W/M428L/M434S/ΔK457 |
| | Hole | C220S/L234A/L235A/P329A/T366S/L368A/Y407V/M428L/M434S/ΔK457 |
| 24 | Knob | L234A/L235A/P329A/S354C/T366W/M428L/M434S/ΔK457 |
| | Hole | L234A/L235A/P329A/Y349C/T366S/L368A/Y407V/M428L/M434S/ΔK457 |
| 25 | Knob | C220S/L234A/L235A/P329A/S354C/T366W/M428L/M434S/ΔK457 |
| | Hole | C220S/L234A/L235A/P329A/Y349C/T366S/L368A/Y407V/M428L/M434S/ΔK457 |
| 26 | Knob | L234A/L235A/P329G/T366W/M428L/M434S/ΔK457 |
| | Hole | L234A/L235A/P329G/T366S/L368A/Y407V/M428L/M434S/ΔK457 |
| 27 | Knob | C220S/L234A/L235G/P329A/T366W/M428L/M434S/ΔK457 |
| | Hole | C220S/L234A/L235G/P329A/T366S/L368A/Y407V/M428L/M434S/ΔK457 |
| 28 | Knob | L234A/L235A/P329G/S354C/T366W/M428L/M434S/ΔK457 |
| | Hole | L234A/L235A/P329G/Y349C/T366S/L368A/Y407V/M428L/M434S/ΔK457 |
| 29 | Knob | C220S/L234A/L235A/P329G/S354C/T366W/M428L/M434S/ΔK457 |
| | Hole | C220S/L234A/L235A/P329G/Y349C/T366S/L368A/Y407V/M428L/M434S/ΔK457 |
| 30 | Knob | L234A/L235E/G237A/A330S/P331S/T366W/M428L/M434S/ΔK457 |
| | Hole | L234A/L235E/G237A/A330S/P331S/T366S/L368A/Y407V/M428L/M434S/ΔK457 |
| 31 | Knob | C220S |
| | | L234A/L235E/G237A/A330S/P331S/T366W/M428L/M434S/ΔK457 |
| | Hole | C220S/L234A/L235E/G237A/A330S/P331S/T366S/L368A/Y407V/M428L/M434S/ΔK457 |
| 32 | Knob | L234A/L235E/G237A/A330S/P331S/S354C/T366W/M428L/M434S/ΔK457 |
| | Hole | L234A/L235E/G237A/A330S/P331S/Y349C/T366S/L368A/Y407V/M428L/M434S/ΔK457 |
| 33 | Knob | C220S/L234A/L235E/G237A/A330S/P331S/S354C/T366W/M428L/M434S/ΔK457 |
| | Hole | C220S/L234A/L235E/G237A/A330S/P331S/Y349C/T366S/L368A/Y407V/ M428L/M434S/ΔK457 |
| 34 | Knob | L234F/L235E/P331S/T366W/M428L/M434S/ΔK457 |
| | Hole | L234F/L235E/P331S/T366S/L368A/Y407V/M428L/M434S/ΔK457 |
| 35 | Knob | C220S/L234F/L235E/P331S/T366W/M428L/M434S/ΔK457 |
| | Hole | C220S/L234F/L235E/P331S//L368A/Y407V/M428L/M434S/ΔK457 |
| 36 | Knob | L234F/L235E/P331S/S354C/T366W/M428L/M434S/ΔK457 |
| | Hole | |
| | | L234F/L235E/P331S/349C/T366S/L368A/Y407V/M428L/M434S/ΔK457 |
| 37 | Knob | C220S/S/L234F/L235E/P331S/354C/T366W/M428L/M434S/ΔK457 |
| | Hole | C220S/L234F/L235E/P331S/Y349C/T366S/L368A/Y407V/M428L/M434S/ΔK457 |

### Modifications to Eliminate Glycosylation Sites

In some embodiments, the Fc domain is modified to eliminate N-linked or O-linked glycosylation sites. A glycosylated variants of Fc domains, particularly of the IgG1 subclass are known to be poor mediators of effector function. Jefferis et al. 1998, Immol. Rev., vol. 163, 50-76). It has been shown that glycosylation at position 297 (EU numbering) contributes to effector function. Edelman, et al (1969) PNAS (USA) 63:78-85. In some embodiments, the Fc domains of the compositions of the present disclosure comprise one or modifications to eliminate N- or O linked glycosylation sites. Examples of modifications at N297 to eliminate glycosylation sites in the Fc domain include the amino acid substitutions N297Q and N297G.

### Albumin Carrier Molecules

In some embodiments, the modified hIL-12p40 polypeptide, hIL-12 mutein, or hIL-23 mutein is conjugated to an albumin molecule (e.g., human serum albumin) which is known in the art to facilitate extended exposure in vivo. In some embodiments, hIL-12p40 polypeptide, hIL-12 mutein, or hIL-23 mutein is conjugated to albumin via chemical linkage or expressed as a fusion protein with an albumin molecule (referred to herein as a “modified hIL-12p40 polypeptide albumin fusion”). The term “albumin” includes albumins such as human serum albumin (HSA), cyno serum albumin, and bovine serum albumin (BSA). In some embodiments, the HSA comprises a C34S or K573P amino acid substitution relative to the wild-type HSA sequence. According to the present disclosure, albumin can be conjugated to a modified hIL-12p40 polypeptide or a hIL-12p35 at the carboxyl terminus, the amino terminus, both the carboxyl and amino termini, and internally (see, e.g., U.S. Pat. Nos. 5,876,969 and 7,056,701). In the HAS-modified polypeptides contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a modified hIL-12p40 polypeptide, hIL-12 mutein, or hIL-23 mutein fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. Such fusion proteins may be readily prepared through recombinant technology to those of ordinary skill in the art. Nucleic acid sequences encoding such fusion proteins may be ordered from any of a variety of commercial sources. The nucleic acid sequence encoding the fusion protein is incorporated into an expression vector operably linked to one or more expression control elements, the vector introduced into a suitable host cell and the fusion protein isolated from the host cell culture by techniques well known in the art.

### Polymeric Carriers

In some embodiments, extended in vivo duration of action of the modified hIL-12p40 polypeptide, hIL-12 mutein, or hIL-23 mutein may be achieved by conjugation to one or more polymeric carrier molecules such as XTEN polymers or water soluble polymers.

### XTEN Conjugates

The modified hIL-12p40 polypeptide, hIL-12 mutein, or hIL-23 mutein may further comprise an XTEN polymer. The XTEN polymer conjugated (either chemically or as a fusion protein) to the modified hIL-12p40 polypeptide or hIL-12 mutein provides extended duration akin to PEGylation and may be produced as a recombinant fusion protein in E. coli. XTEN polymers suitable for use in conjunction with the hIL-12p40 polypeptide, hIL-12 mutein, or hIL-23 mutein of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease-Activatable Killin-Based Cytostatic” PLOS ONE DOI: 10.1371/journal.pone.0157193 Jun. 13, 2016. The XTEN polymer fusion protein may incorporate a protease sensitive cleavage site between the XTEN polypeptide and the hIL2 mutein such as an MMP-2 cleavage site.

### Water Soluble Polymers

In some embodiments, the modified hIL-12p40 polypeptide, hIL-12 mutein, or hIL-23 muteincan be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly (oxyethylated polyol), polyolefinic alcohol,), polysaccharides), poly-alpha-hydroxy acid), polyvinyl alcohol (PVA), polyphosphazene, polyoxazolines (POZ), poly(N-acryloylmorpholine), or a combination thereof.

In some embodiments, the modified hIL-12p40 polypeptide, hIL-12 mutein, hIL-12 Fc mutein, or hIL-23 mutein can be conjugated to one or more polyethylene glycol molecules or “PEGylated.” In some embodiments, the hIL-12p40 polypeptide of the hIL-12 mutein or hIL-12 Fc mutein is PEGylated. In some embodiments, the hIL-12p35 of the hIL-12 mutein or hIL-12 Fc mutein is PEGylated. In some embodiments, the hIL-12p40 polypeptide of the hIL-23 mutein is PEGylated. In some embodiments, the human p19 polypeptide of the hIL-23 mutein is PEGylated. Although the method or site of PEG attachment to the binding molecule may vary, in certain embodiments the PEGylation does not alter, or only minimally alters, the activity of the binding molecule.

PEGs suitable for conjugation to a polypeptide sequence are generally soluble in water at room temperature, and have the general formula

$${R ( O - CH 2 - CH 2 )}_{n} O - R ,$$

where R is hydrogen or a protective group such as an alkyl or an alkanol group, and where n is an integer from 1 to 1000. When R is a protective group, it generally has from 1 to 8 carbons. The PEG can be linear or branched. Branched PEG derivatives, “star-PEGs” and multi-armed PEGs are contemplated by the present disclosure.

In some embodiments, selective PEGylation of the modified In some embodiments, the hIL-12p40 of the hIL-12 mutein is PEGylated. In some embodiments, the hIL-12p35 of the hIL-12 mutein is PEGylated., for example, by the incorporation of non-natural amino acids having side chains to facilitate selective PEG conjugation, may be employed. Specific PEGylation sites can be chosen such that PEGylation of the binding molecule does not affect its binding to the target receptors.

In certain embodiments, the increase in half-life is greater than any decrease in biological activity. PEGs suitable for conjugation to a polypeptide sequence are generally soluble in water at room temperature, and have the general formula R(O-CH2-CH2)nO-R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding molecule can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O-CH2-CH2)nO-R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding molecule of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10kDA linear PEG molecules (e.g., Sunbright® GL2-200AL3, NOF), a 20 kDa 2-arm branched PEG-NHS ester the 20 kDA PEG-NHS ester comprising two 10kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20kDA linear PEG molecules (e.g., Sunbright® GL2-400AL3), a 40 kDa 2-arm branched PEG-NHS ester the 40 kDA PEG-NHS ester comprising two 20kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

In some embodiments, a linker can used to join the modified hIL-12p40 polypeptide or hIL-12 mutein, and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding molecule is acetylated at the N-terminus by enzymatic reaction with N-terminal acetyltransferase and, for example, acetyl COA. Alternatively, or in addition to N-terminal acetylation, the binding molecule can be acetylated at one or more lysine residues, e.g., by enzymatic reaction with a lysine acetyltransferase. See, for example Choudhary et al. (2009) Science 325 (5942): 834 840.

In some embodiments, the present disclosure provides a modified hIL-12p40 polypeptide that is PEGylated, wherein the PEG is conjugated to the modified hIL-12p40 polypeptide and the PEG is a linear or branched PEG molecule having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms. In some embodiments, the PEG is conjugated to the N-terminus of the modified hIL-12p40 polypeptide of an hIL-12 mutein comprising a modified hIL-12p40 polypeptide.

In some embodiments, the PEG is conjugated to the C-terminus of the modified hIL-12p40 polypeptide of an hIL-12 mutein comprising a modified hIL-12p40 polypeptide. In some embodiments, the carrier molecule is conjugated to the modified hIL-12p40 polypeptide of an hIL-12 mutein via a linker. In some embodiments, the PEG is conjugated to the N-terminus of the hIL-12p35 subunit of an hIL-12 mutein comprising a modified hIL-12p40 polypeptide. In some embodiments, the PEG is conjugated to the C-terminus of the hIL-12p35 subunit of an hIL-12 mutein comprising a modified hIL-12p40 polypeptide. In some embodiments, the carrier molecule is conjugated to the hIL-12p35 subunit of an hIL-12 mutein via a linker.

In some embodiments, the PEG is conjugated to the C-terminus of the modified hIL-12p40 polypeptide of an hIL-23 mutein comprising a modified hIL-12p40 polypeptide. In some embodiments, the carrier molecule is conjugated to the modified hIL-12p40 polypeptide of an hIL-23 mutein via a linker. In some embodiments, the PEG is conjugated to the N-terminus of the human p19 subunit of an hIL-23 mutein comprising a modified hIL-12p40 polypeptide. In some embodiments, the PEG is conjugated to the C-terminus of the human p19 subunit of an hIL-23 mutein comprising a modified hIL-12p40 polypeptide. In some embodiments, the carrier molecule is conjugated to the human p19 subunit of an hIL-23 mutein via a linker.

### Modifications to Provide Additional Functions

In some embodiments, the modified hIL-12p40 polypeptide may comprise a functional domain of a chimeric polypeptide. Modified hIL-12p40 polypeptide fusion proteins of the present disclosure may be readily produced by recombinant DNA methodology by techniques known in the art. These methods comprise constructing a recombinant vector comprising a nucleic acid sequence comprising a nucleic acid sequence encoding the modified hIL-12p40 polypeptide in frame with a nucleic acid sequence encoding the fusion partner either at the N-terminus or C-terminus of the modified hIL-12p40 polypeptide. Optionally, the nucleic acid sequence encoding the modified hIL-12p40 polypeptide and the nucleic acid encoding the fusion partner are separated by a nucleic acid sequence encoding a linker.

### FLAG Tags

In some embodiments, the modified hIL-12p40 polypeptide can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding molecule further comprises a C-terminal c-myc epitope tag.

### Targeting Moieties

In some embodiments, the modified hIL-12p40 polypeptide is conjugated to a molecule (“targeting domain”) to facilitate selective binding to particular cell type or tissue expressing a cell surface molecule that specifically binds to such targeting domain, optionally incorporating a linker molecule of from 1-40 (alternatively 2-20, alternatively 5-20, alternatively 10-20) amino acids between the modified IL-12p40 polypeptide sequence and the sequence of the targeting domain of the fusion protein. In some embodiments, an hIL-12 mutein comprising a modified hIL-12p40 polypeptide is conjugated to a targeting domain, by attaching the targeting sequence to the hIL-12p40 polypeptide or attaching the targeting sequence to the hIL-12p35 polypeptide.

In other embodiments, a chimeric polypeptide including a modified hIL-12p40 polypeptide, and an antibody or antigen-binding portion thereof can be generated. The antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting moiety is an antibody that specifically binds to at least one cell surface molecule associated with a tumor cell (i.e. at least one tumor antigen).

### Recombinant Production

In some embodiments, the hIL-12p40 molecules, hIL-12 muteins or hIL-23 muteins of the present disclosure are produced by recombinant DNA technology. In the typical practice of recombinant production of polypeptides, a nucleic acid sequence encoding the desired polypeptide(s) is incorporated into an expression vector suitable for the host cell in which expression will be accomplished, the nucleic acid sequence being operably linked to one or more expression control sequences encoded by the vector and functional in the target host cell. The recombinant protein may be recovered through disruption of the host cell or from the cell medium if a secretion leader sequence (signal peptide) is incorporated into the polypeptide.

Nucleic Acid Sequences Encoding Modified hIL-12p40 Polypeptides

In some embodiments, the modified hIL-12p40polypeptide is produced by recombinant methods using a nucleic acid sequence encoding the modified hIL-12p40 polypeptide (or fusion protein comprising the modified hIL-12p40 polypeptide). In some embodiments, a hIL-12 mutein described herein is produced by recombinant methods using a nucleic acid sequence encoding the modified hIL-12p40 polypeptide and a nucleic acid sequence encoding a hIL-12p35 polypeptide. Similarly, a hIL-23 mutein is produced by recombinant methods using a nucleic acid sequence encoding the modified hIL-12p40 polypeptide and a nucleic acid sequence encoding a human p19 polypeptide. In some embodiments, the hIL-12 mutein is a single chain hIL-12 mutein encoded by a nucleic acid sequence comprising a nucleic acid sequence encoding the modified hIL-12p40 polypeptide and a nucleic acid encoding the hIL-12p35 polypeptide, wherein the modified hIL-12p40 polypeptide and the hIL-12p35 are linked via a peptide linker. In some embodiments, the hIL-23 mutein is a single chain hIL-23 mutein encoded by a nucleic acid sequence comprising a nucleic acid sequence encoding the modified hIL-12p40 polypeptide and a nucleic acid encoding the human p19 polypeptide, wherein the modified hIL-12p40 polypeptide and the human p19 polypeptide are linked via a peptide linker.

In some embodiments, the single-chain hIL-12 mutein is produced using a multicistronic vector (e.g., a bicistronic vector) encoding the modified hIL-12p40 polypeptide and the nucleic acid encoding the hIL-12p35 polypeptide, wherein the modified hIL-12p40 polypeptide and the hIL-12p35 are linked by a coding sequence for a self-cleaving peptide. In some embodiments, the single-chain hIL-23 mutein is produced using a multicistronic vector (e.g., a bicistronic vector) encoding the modified hIL-12p40 polypeptide and the nucleic acid encoding the human p19 polypeptide, wherein the modified hIL-12p40 polypeptide and the human p19 polypeptide are linked by a coding sequence for a self-cleaving peptide. Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Lieschke et al., and Chng et al. “Cleavage efficient 2A peptides for high level monoclonal antibody expression in CHO cells,” MAbs 7 (2): 403-412 (2015)).

The nucleic acid sequence encoding the desired modified hIL-12p40 polypeptide can be synthesized by chemical means using an oligonucleotide synthesizer. The disclosure also provides nucleic acid molecules that encode the modified hIL-12p40 polypeptide having an amino acid substitution described herein described herein. In some embodiments, the nucleic acid molecule comprises a nucleic acid sequence encoding a mature form of an hIL-12p40 peptide, i.e, without the signal peptide sequence. In some embodiments, the nucleic acid molecule comprises a nucleic acid sequence encoding an hIL-12p40 polypeptide having at least 70% sequence identity to SEQ ID NO: 1 (e.g., at least 70%, 75%, 80%, 85%, 90%, 91%, 92%, 93%, 94%, 95%, 96%, 97%, 98%, or 99% sequence identity to SEQ ID NO:1), and comprises two or more amino acid substitutions, wherein the polypeptide comprises amino acid substitutions at positions corresponding to amino acid residues E81 and F82 of SEQ ID NO: 1, wherein (a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or (b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y). In some embodiments, the hIL-12p40 polypeptide further comprises one or more amino acid substitutions at one or more positions corresponding to an amino acid residue selected from the group consisting of W37, P39, D40, A41, Q64, K80, A85, E108, D115, H216, K217, L218, and K219 of SEQ ID NO: 1, wherein the amino acid substitution can be any amino acid.

In some embodiments, the nucleic acid molecule further comprises a nucleic acid sequence encoding a signal peptide. In some embodiments, the signal peptide comprises an endogenous or wild-type hIL-12p40 signal peptide. In some embodiments, the signal peptide comprises the amino acid sequence of the human hIL-12p40 polypeptide: MCHQQLVISWFSLVFLASPLVA (SEQ ID NO:2).

In some embodiments, the modified hIL-12p40 polypeptide is produced by recombinant methods using a nucleic acid sequence encoding the modified hIL-12p40 polypeptide (or fusion protein comprising the modified hIL-12p40 polypeptide). The nucleic acid sequence encoding the desired modified hIL-12p40 polypeptide can be synthesized by chemical means using an oligonucleotide synthesizer.

The nucleic acid molecules are not limited to sequences that encode polypeptides; some or all of the non-coding sequences that lie upstream or downstream from a coding sequence (e.g., the coding sequence of hIL-12p40) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The nucleic acid molecules encoding the modified hIL-12p40 polypeptide (and fusions thereof) may contain naturally occurring sequences or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

Nucleic acid sequences encoding the modified hIL-12p40 polypeptide may be obtained from various commercial sources that provide custom made nucleic acid sequences. Amino acid sequence variants of the modified hIL-12p40 polypeptide of the present disclosure are prepared by introducing appropriate nucleotide changes into the coding sequence based on the genetic code which is well known in the art. Such variants represent insertions, substitutions, and/or specified deletions of, residues as noted. Any combination of insertion, substitution, and/or specified deletion is made to arrive at the final construct, provided that the final construct possesses the desired biological activity as defined herein.

Methods for constructing a DNA sequence encoding a modified hIL-12p40 polypeptide and expressing those sequences in a suitably transformed host include, but are not limited to, using a PCR-assisted mutagenesis technique. Mutations that consist of deletions or additions of amino acid residues to a modified hIL-12p40 polypeptide can also be made with standard recombinant techniques. In the event of a deletion or addition, the nucleic acid molecule encoding a modified hIL-12p40 polypeptide is optionally digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

A modified hIL-12p40 polypeptide of the present disclosure may be produced recombinantly not only directly, but also as a fusion polypeptide with a heterologous polypeptide, e.g. a signal sequence or other polypeptide having a specific cleavage site at the N-terminus or C-terminus of the mature modified hIL-12p40 polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. The inclusion of a signal sequence depends on whether it is desired to secrete the modified hIL-12p40 polypeptide from the recombinant cells in which it is made. If the chosen cells are prokaryotic, it generally is preferred that the DNA sequence not encode a signal sequence. When the recombinant host cell is a yeast cell such as Saccharomyces cerevisiae, the alpha mating factor secretion signal sequence may be employed to achieve extracellular secretion of the modified hIL-12p40 polypeptide into the culture medium as described in Singh, U.S. Pat. No. 7,198,919 B1 issued Apr. 3, 2007.

When the hIL-12p40 polypeptide is expressed as a chimera (e.g., a fusion protein comprising a modified hIL-12p40 polypeptide and a heterologous polypeptide sequence), the chimeric protein can be encoded by a hybrid nucleic acid molecule comprising a first sequence that encodes all or part of the modified hIL-12p40 polypeptide and a second sequence that encodes all or part of the heterologous polypeptide. For example, subject modified hIL-12p40 polypeptides described herein may be fused to a hexa-/octa-histidine tag to facilitate purification of bacterially expressed protein, or to a hemagglutinin tag to facilitate purification of protein expressed in eukaryotic cells. By first and second, it should not be understood as limiting to the orientation of the elements of the fusion protein and a heterologous polypeptide can be linked at either the N-terminus and/or C-terminus of the modified hIL-12p40 polypeptide. For example, the N-terminus may be linked to a targeting domain and the C-terminus linked to a hexa-histidine tag purification handle.

The complete amino acid sequence of the polypeptide (or fusion/chimera) to be expressed can be used to construct a back-translated gene. A DNA oligomer containing a nucleotide sequence coding a modified hIL-12p40 polypeptide can be synthesized. For example, several small oligonucleotides coding for portions of the desired polypeptide can be synthesized and then ligated. The individual oligonucleotides typically contain 5′ or 3′ overhangs for complementary assembly.

### Codon Optimization

In some embodiments, the nucleic acid sequence encoding the modified hIL-12p40 polypeptide may be “codon optimized” to facilitate expression in a particular host cell type. Techniques for codon optimization in a wide variety of expression systems, including mammalian, yeast and bacterial host cells, are well known in the and there are online tools to provide for a codon optimized sequences for expression in a variety of host cell types. See e.g. Hawash, et al., (2017) 9:46-53 and Mauro and Chappell in Recombinant Protein Expression in Mammalian Cells: Methods and Protocols, edited by David Hacker (Human Press New York). Additionally, there are a variety of web based on-line software packages that are freely available to assist in the preparation of codon optimized nucleic acid sequences.

### Expression Vectors

Once assembled (by synthesis, site-directed mutagenesis or another method), the nucleic acid sequence encoding an a modified hIL-12p40 polypeptide, hIL-12 mutein or hIL-23 mutein will be inserted into an expression vector. A variety of expression vectors for uses in various host cells are available and are typically selected based on the host cell for expression. An expression vector typically includes, but is not limited to, one or more of the following: an origin of replication, one or more marker genes, an enhancer element, a promoter, and a transcription termination sequence. Vectors include viral vectors, plasmid vectors, integrating vectors, and the like. Plasmids are examples of non-viral vectors.

To facilitate efficient expression of the recombinant polypeptide, the nucleic acid sequence encoding the polypeptide sequence to be expressed is operably linked to transcriptional and translational regulatory control sequences that are functional in the chosen expression host.

### Selectable Marker

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

### Regulatory Control Sequences

Expression vectors for a modified hIL-12p40 polypeptide of the present disclosure can contain a regulatory sequence that is recognized by the host organism and is operably linked to a nucleic acid sequence encoding the modified hIL-12p40 polypeptide. The terms “regulatory control sequence,” “regulatory sequence” or “expression control sequence” are used interchangeably herein to refer to promoters, enhancers, and other expression control elements (e.g., polyadenylation signals). See, for example, Goeddel (1990) in Gene Expression Technology: Methods in Enzymology 185 (Academic Press, San Diego CA USA Regulatory sequences include those that direct constitute expression of a nucleotide sequence in many types of host cells and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. In selecting an expression control sequence, a variety of factors understood by one of skill in the art are to be considered. These include, for example, the relative strength of the sequence, its controllability, and its compatibility with the actual DNA sequence encoding the subject a modified hIL-12p40 polypeptide, particularly as regards potential secondary structures.

### Promoters

In some embodiments, the regulatory sequence is a promoter, which is selected based on, for example, the cell type in which expression is sought. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

A T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

Transcription from vectors in mammalian host cells may be controlled, for example, by promoters obtained from the genomes of viruses such as polyoma virus, fowlpox virus, adenovirus (such as human adenovirus serotype 5), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

### Enhancers

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter. Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In addition to sequences that facilitate transcription of the inserted nucleic acid molecule, vectors can contain origins of replication, and other genes that encode a selectable marker. For example, the neomycin-resistance (neoR) gene imparts G418 resistance to cells in which it is expressed, and thus permits phenotypic selection of the transfected cells. Additional examples of marker or reporter genes include beta-lactamase, chloramphenicol acetyltransferase (CAT), adenosine deaminase (ADA), dihydrofolate reductase (DHFR), hygromycin-B-phosphotransferase (HPH), thymidine kinase (TK), lacZ (encoding beta-galactosidase), and xanthine guanine phosphoribosyltransferase (XGPRT). Those of skill in the art can readily determine whether a given regulatory element or selectable marker is suitable for use in a particular experimental context.

Proper assembly of the expression vector can be confirmed by nucleotide sequencing, restriction mapping, and expression of a biologically active polypeptide in a suitable host.

### Host Cells

The present disclosure further provides prokaryotic or eukaryotic cells that contain and express a nucleic acid molecule that encodes a modified hIL-12p40 polypeptide, hIL-12 mutein or hIL-23 mutein. A cell of the present disclosure is a transfected cell, i.e., a cell into which a nucleic acid molecule, for example a nucleic acid molecule encoding a modified hIL-12p40 polypeptide, hIL-12 mutein or hIL-23 mutein, has been introduced by means of recombinant DNA techniques. In some embodiments, the cell expresses a hIL-12p35 polypeptide such that, when the hIL12-p40 polypeptide is expressed, the modified hIL-12p40 polypeptide associates with the hIL-12p35 polypeptide to form a hIL-12 mutein. In some embodiments, the cell comprises a nucleic acid sequence encoding hIL-12p35 polypeptide and a nucleic acid sequence encoding the modified hIL-12p40 polypeptide. In some embodiments, the cell expresses a human p19 polypeptide such that, when the hIL 12-p40 polypeptide is expressed, the modified hIL-12p40 polypeptide associates with the human p19 polypeptide to form a hIL-23 mutein. In some embodiments, the cell comprises a nucleic acid sequence encoding a hunan p19 polypeptide and a nucleic acid sequence encoding the modified hIL-12p40 polypeptide. The progeny of such a cell are also considered within the scope of the present disclosure.

Host cells are typically selected in accordance with their compatibility with the chosen expression vector, the toxicity of the product coded for by the DNA sequences of this invention, their secretion characteristics, their ability to fold the polypeptides correctly, their fermentation or culture requirements, and the ease of purification of the products coded for by the DNA sequences. Suitable host cells for cloning or expressing the DNA in the vectors herein are the prokaryote, yeast, or higher eukaryote cells.

In some embodiments the recombinant modified hIL-12p40 polypeptide or hIL-12 mutein can also be made in eukaryotes, such as yeast or human cells. Suitable eukaryotic host cells include insect cells (examples of Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39)); yeast cells (examples of vectors for expression in yeast S. cerevisiae include pYepSecl (Baldari et al. (1987) EMBO J. 6:229-234), pMFa (Kurjan and Herskowitz (1982) Cell 30:933-943), pJRY88 (Schultz et al. (1987) Gene 54:113-123), pYES2 (Invitrogen Corporation, San Diego, Calif.), and pPicZ (Invitrogen Corporation, San Diego, Calif.)); or mammalian cells (mammalian expression vectors include pCDM8 (Seed (1987) Nature 329:840) and pMT2PC (Kaufman et al. (1987) EMBO J. 6:187:195)).

Examples of useful mammalian host cell lines are mouse L cells (L-M [TK-], ATCC #CRL-2648), monkey kidney CVI line transformed by SV40 (COS-7, ATCC CRL 1651); human embryonic kidney line (HEK293 or HEK293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2). In mammalian cells, the expression vector's control functions are often provided by viral regulatory elements. For example, commonly used promoters are derived from polyoma, Adenovirus 2, cytomegalovirus, and Simian Virus 40.

The modified hIL-12p40 polypeptide, hIL-12 mutein or hIL-23 mutein may be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

In some embodiments, a modified hIL-12p40 polypeptide, hIL-12 mutein or hIL-23 mutein obtained will be glycosylated or unglycosylated depending on the host organism used. If bacteria are chosen as the host then the modified hIL-12p40 polypeptide, hIL-12 mutein or hIL-23 mutein produced will be unglycosylated. Eukaryotic cells, on the other hand, will typically result in glycosylation of the modified hIL-12p40 polypeptide, hIL-13 mutein or hIL-23 mutein.

For other additional expression systems for both prokaryotic and eukaryotic cells, see Chapters 16 and 17 of Sambrook et al. (1989) Molecular Cloning: A Laboratory Manual (2nd ed., Cold Spring Harbor Laboratory Press, Plainview, N.Y.). See, Goeddel (1990) in Gene Expression Technology: Methods in Enzymology 185 (Academic Press, San Diego, Calif.).

### Transfection

The expression constructs of the present diclsoure can be introduced into host cells to thereby produce a modified hIL-12p40 polypeptide ofr IL-12 mutein disclosed herein. The expression vector comprising a nucleic acid sequence encoding modified hIL-12p40 polypeptide is introduced into the prokaryotic or eukaryotic host cells via conventional transformation or transfection techniques. Suitable methods for transforming or transfecting host cells can be found in Sambrook et al. (1989) Molecular Cloning: A Laboratory Manual (2d ed., Cold Spring Harbor Laboratory Press, Plainview, N.Y.) and other standard molecular biology laboratory manuals. To facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, and magnetic fields (electroporation).

### Cell Culture

Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

### Recovery of Recombinant Proteins

Recombinantly produced modified hIL-12p40 polypeptides or hIL-12 muteins can be recovered from the culture medium as a secreted polypeptide if a secretion leader sequence is employed. Alternatively, the modified hIL-12p40 polypeptide, hIL-13 mutein or hIL-23 mutein can also be recovered from host cell lysates. A protease inhibitor, such as phenyl methyl sulfonyl fluoride (PMSF) may be employed during the recovery phase from cell lysates to inhibit proteolytic degradation during purification, and antibiotics may be included to prevent the growth of adventitious contaminants.

Various purification steps are known in the art and find use, e.g. affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural specific binding of one molecular species to separate and purify a second species from a mixture. Antibodies are commonly used in affinity chromatography. Size selection steps may also be used, e.g. gel filtration chromatography (also known as size-exclusion chromatography or molecular sieve chromatography) is used to separate proteins according to their size. In gel filtration, a protein solution is passed through a column that is packed with semipermeable porous resin. The semipermeable resin has a range of pore sizes that determines the size of proteins that can be separated with the column.

A recombinantly modified hIL-12p40 polypeptide, hIL-13 mutein or hIL-23 mutein expressed by the transformed host can be purified according to any suitable method. Recombinant modified hIL-12p40 polypeptides or hIL-12 muteins can be isolated from inclusion bodies generated in E. coli, or from conditioned medium from either mammalian or yeast cultures producing a given mutein using cation exchange, gel filtration, and or reverse phase liquid chromatography. The substantially purified forms of the recombinant modified hIL-12p40 polypeptide, hIL-13 mutein or hIL-23 mutein can be purified from the expression system using routine biochemical procedures, and can be used, e.g., as therapeutic agents, as described herein.

In some embodiments, where the modified hIL-12p40 polypeptide, hIL-13 mutein or hIL-23 mutein is expressed with a purification tag as discussed above, this purification handle may be used for isolation of the modified hIL-12p40 polypeptide, hIL-13 mutein or hIL-23 mutein from the cell lysate or cell medium. Where the purification tag is a chelating peptide, methods for the isolation of such molecules using immobilized metal affinity chromatography are well known in the art. See, e.g., Smith, et al. U.S. Pat. No. 4,569,794.

The biological activity of the modified hIL-12p40 polypeptide, hIL-13 mutein or hIL-23 mutein recovered can be assayed for activity by any suitable method known in the art and may be evaluated as substantially purified forms or as part of the cell lysate or cell medium when secretion leader sequences are employed for expression.

### Pharmaceutical Formulations

In some embodiments, the subject modified hIL-12p40 polypeptide (and/or nucleic acids encoding the modified hIL-12p40 polypeptide or recombinant cells incorporating a nucleic acid sequence and modified to express the modified hIL-12p40 polypeptide) can be incorporated into compositions, including pharmaceutical compositions. In some embodiments, the pharmaceutical composition comprises a hIL 12 mutein or hIL-23 mutein comprising a modified hIL-12p40 polypeptide described herein. Such compositions typically include the polypeptide or nucleic acid molecule and a pharmaceutically acceptable carrier. A pharmaceutical composition is formulated to be compatible with its intended route of administration and is compatible with the therapeutic use for which the modified hIL-12p40 polypeptide or hIL-12 mutein is to be administered to the subject in need of treatment or prophylaxis.

### Carriers

Carriers include a sterile diluent such as water for injection, saline solution, fixed oils, polyethylene glycols, glycerine, propylene glycol or other synthetic solvents. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. For intravenous administration, suitable carriers include physiological saline, bacteriostatic water, Cremophor EL™ (BASF, Parsippany, N.J.) or phosphate buffered saline (PBS).

### Buffers

The term buffers includes buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5).

### Dispersions

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### Preservatives

The pharmaceutical formulations for parenteral administration to a subject should be sterile and should be fluid to facilitate easy syringability. It should be stable under the conditions of manufacture and storage and are preserved against the contamination. Prevention of the action of microorganisms can be achieved by various antibacterial and antifungal agents, for example, agents such as benzyl alcohol or methyl parabens; antioxidants such as ascorbic acid or sodium bisulfite; chelating agents such as ethylenediaminetetraacetic acid, parabens, chlorobutanol, phenol, ascorbic acid, thimerosal, and the like. Sterile solutions can be prepared by incorporating the active compound in the required amount in an appropriate solvent with one or a combination of ingredients enumerated above, as required, followed by filtered sterilization.

### Tonicity Agents

In many cases, it will be preferable to include isotonic agents, for example, sugars, polyalcohols such as mannitol, sorbitol, sodium chloride in the composition.

### Routes of Administration

In some embodiments of the therapeutic methods of the present disclosure involve the administration of a pharmaceutical formulation comprising a modified hIL-12p40 polypeptide (and/or nucleic acids encoding the modified hIL-12p40 polypeptide or recombinantly modified host cells expressing the modified hIL-12p40 polypeptide) to a subject in need of treatment. In other embodiments, the therapeutic methods of the present disclosure involve the administration of a pharmaceutical formulation comprising a hIL-12 mutein comprising a modified hIL-12p40 polypeptide described herein. Any of the pharmaceutical compositions of the present disclosure may be administered to a subject in need of treatment or prophyaxis by a variety of routes of administration, including parenteral administration, oral, topical, or inhalation routes.

### Parenteral Administration

In some embodiments, the methods of the present disclosure involve the parenteral administration of a pharmaceutical formulation comprising a modified hIL-12p40 polypeptide (and/or nucleic acids encoding the modified hIL-12p40 polypeptide or recombinantly modified host cells expressing the modified hIL-12p40 polypeptide) to a subject in need of treatment. In some embodiments, the methods of the present disclosure involve the parenteral administration of a pharmaceutical formulation comprising a hIL-12 mutein or hIL-23 mutein comprising a modified hIL-12p40 polypeptide to a subject in need of treatment. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe.

### Oral Administration

In some embodiments, the methods of the present disclosure involve the oral administration of a pharmaceutical formulation comprising a modified hIL-12p40 polypeptide (and/or nucleic acids encoding the modified hIL-12p40 polypeptide or recombinantly modified host cells expressing the modified hIL-12p40 polypeptide) to a subject in need of treatment. In some embodiments, the methods of the present disclosure involve the oral administration of a pharmaceutical formulation comprising a hIL-12 mutein or hIL-23 mutein comprising a modified hIL-12p40 polypeptide to a subject in need of treatment. Oral compositions, if used, generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

### Inhalation Formulations

In some embodiments, the methods of the present disclosure involve the inhaled administration of a pharmaceutical formulation comprising a modified hIL-12p40 polypeptide (and/or nucleic acids encoding the modified hIL-12p40 polypeptide or recombinantly modified host cells expressing the modified hIL-12p40 polypeptide) to a subject in need of treatment. In some embodiments, the methods of the present disclosure involve the inhaled administration of a pharmaceutical formulation comprising a hIL-12 mutein or hIL-23 mutein comprising a modified hIL-12p40 polypeptide to a subject in need of treatment. In the event of administration by inhalation, hIL-12 muteins, hIL-23 muteins, subject modified hIL-12p40 polypeptides, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

### Mucosal and Transdermal Formulations

In some embodiments, the methods of the present disclosure involve the mucosal or transdermal administration of a pharmaceutical formulation comprising a modified hIL-12p40 polypeptide (and/or nucleic acids encoding the modified hIL-12p40 polypeptide or recombinantly modified host cells expressing the modified hIL-12p40 polypeptide) to a subject in need of treatment. In some embodiments, the methods of the present disclosure involve the mucosal or transdermal administration of a pharmaceutical formulation comprising a hIL-12 mutein or hIL-23 comprising a modified hIL-12p40 polypeptide to a subject in need of treatment. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

### Extended Release and Depot Formulations

In some embodiments of the method of the present disclosure, the modified hIL-12p40 polypeptide is administered to a subject in need of treatment in a formulation to provide extended release of the modified hIL-12p40 polypeptide, hIL-12 mutein comprising the modified hIL-12p40 polypeptide, or hIL-23 comprising the modified hIL-12p40 polypeptide. Examples of extended release formulations of the injectable compositions can be brought about by including in the composition an agent which delays absorption, for example, aluminum monostearate and gelatin. In one embodiment, the subject modified hIL-12p40 polypeptide, hIL-12 mutein, hIL-23 mutein or nucleic acids encoding them are prepared with carriers that will protect the modified hIL-12p40 polypeptide, hIL-12 mutein or hIL-23 mutein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Administration of Nucleic Acids Encoding the Modified hIL-12p40 Polypeptide

In some embodiments of the method of the present disclosure, delivery of the modified hIL-12p40 polypeptide to a subject in need of treatment is achieved by the administration of a nucleic acid encoding the modified hIL-12p40 polypeptide, hIL 12 mutein or hIL-23 mutein. Methods for the administration of a nucleic acid encoding the modified hIL-12p40 polypeptide, hIL 12 mutein, or hIL-23 mutein to a subject is achieved by transfection or infection using methods known in the art, including but not limited to the methods described in McCaffrey et al. (Nature (2002) 418:6893), Xia et al. (Nature Biotechnol. (2002) 20:1006-1010), or Putnam (Am. J. Health Syst. Pharm. (1996) 53:151-160 erratum at Am. J. Health Syst. Pharm. (1996) 53:325). In some embodiments, the modified hIL-12p40 polypeptide, hIL 12 mutein or hIL-23 mutein is administered to a subject by the administration of a pharmaceutically acceptable formulation of recombinant expression vector comprising a nucleic acid sequence encoding the modified hIL-12p40 polypeptide, hIL-12 mutein or hIL-23 mutein operably linked to one or more expression control sequences operable in a mammalian subject. In some embodiments, the expression control sequence may be selected that is operable in a limited range of cell types (or single cell type) to facilitate the selective expression of the modified hIL-12p40 polypeptide, hIL 12 mutein or hIL-23 mutein in a particular target cell type. In one embodiment, the recombinant expression vector is a viral vector. In some embodiments, the recombinant vector is a recombinant viral vector. In some embodiments the recombinant viral vector is a recombinant adenoassociated virus (rAAV) or recombinant adenovirus (rAd), in particular a replication deficient adenovirus derived from human adenovirus serotypes 3 and/or 5. In some embodiments, the replication deficient adenovirus has one or more modifications to the El region which interfere with the ability of the virus to initiate the cell cycle and/or apoptotic pathways in a human cell. The replication deficient adenoviral vector may optionally comprise deletions in the E3 domain. In some embodiments the adenovirus is a replication competent adenovirus. In some embodiments the adenovirus is a replication competent recombinant virus engineered to selectively replicate in the target cell type.

In some embodiments, particularly for administration of modified hIL-12p40 polypeptides, hIL-12 muteins or hIL-23 muteins to the subject, the nucleic acid encoding the modified hIL-12p40 polypeptide, hIL-12 mutein or hIL-23 mutein may be delivered to the subject by the administration of a recombinantly modified bacteriophage vector encoding the modified hIL-12p40 polypeptide, hIL-12 mutein or hIL-23 mutein. As used herein, the terms “prokaryotic virus,” “bacteriophage” and “phage” are used interchangeably hereinto describe any of a variety of bacterial viruses that infect and replicate within a bacterium. Bacteriophage selectively infect prokaryotic cells, restricting the expression of the modified hIL-12p40 polypeptide, hIL12 mutein or hIL-23 mutein to prokaryotic cells in the subject while avoiding expression in mammalian cells. A wide variety of bacteriophages capable of selection a broad range of bacterial cells have been identified and characterized extensively in the scientific literature. In some embodiments, the phage is modified to remove adjacent motifs (PAM). Elimination of the of Cas9 sequences from the phage genome reduces ability of the Cas9 endonuclease of the target prokaryotic cell to neutralize the invading phage encoding the modified hIL-12p40 polypeptide, hIL-12 mutein or hIL-23 mutein.

Administration of Recombinantly Modified Cells Expressing the Modified hIL-12p40 Polypeptide

In some embodiments of the method of the present disclosure, delivery of the modified hIL-12p40 polypeptide to a subject in need of treatment is achieved by the administration of recombinant host cells modified to express the modified hIL-12p40 polypeptide, hIL 12 mutein or hIL-23 mutein, which may be administered in the therapeutic and prophylactic applications described herein. In some embodiments, the recombinant host cells are mammalian cells, e.g., human cells.

In some embodiments, the nucleic acid sequence encoding the modified hIL-12p40 polypeptide, hIL12 mutein, or hIL-23 mutein (or vectors comprising same) may be maintained extrachromosomally in the recombinantly modified host cell for administration. In other embodiments, the nucleic acid sequence encoding the modified hIL-12p40 polypeptide, hIL12 mutein, or hIL-23 mutein may be incorporated into the genome of the host cell to be administered using at least one endonuclease to facilitate incorporate insertion of a nucleic acid sequence into the genomic sequence of the cell. As used herein, the term “endonuclease” is used to refer to a wild-type or variant enzyme capable of catalyzing the cleavage of bonds between nucleic acids within a DNA or RNA molecule, preferably a DNA molecule. Endonucleases are referred to as “rare-cutting” endonucleases when such endonucleases have a polynucleotide recognition site greater than about 12 base pairs (bp) in length, more preferably of 14-55 bp. Rare-cutting endonucleases can be used for inactivating genes at a locus or to integrate transgenes by homologous recombination (HR) i.e. by inducing DNA double-strand breaks (DSBs) at a locus and insertion of exogenous DNA at this locus by gene repair mechanism. Examples of rare-cutting endonucleases include homing endonucleases (Grizot, et al (2009) Nucleic Acids Research 37 (16): 5405-5419), chimeric Zinc-Finger nucleases (ZFN) resulting from the fusion of engineered zinc-finger domains (Porteus M and Carroll D., Gene targeting using zinc finger nucleases (2005) Nature Biotechnology 23 (3): 967-973, a TALEN-nuclease, a Cas9 endonuclease from CRISPR system as or a modified restriction endonuclease to extended sequence specificity (Eisenschmidt, et al. 2005; 33 (22): 7039-7047).

### Methods of Use

### Treatment of Neoplastic Disease

The present disclosure provides methods of use of hIL 12 muteins of the present disclosure in the treatment of subjects suffering from a neoplastic disease disorder or condition by the administration of a therapeutically effective amount of a hIL 12 mutein (or nucleic acid encoding a hIL 12 mutein or hIL 12p40 polypeptide including recombinant vectors encoding hIL12 mutein or hIL12p40 polypeptide, and eukaryotic and prokaryotic cells modified to express a hIL 12 mutein or hIL12p40 polypeptide) as described herein.

Neoplasms amenable to treatment:

The compositions and methods of the present disclosure are useful in the treatment of subject suffering from a neoplastic disease characterized by the presence neoplasms, including benign and malignant neoplasms, and neoplastic disease.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

Combination Of hIL 12 muteins with Supplementary Anti-Neoplastic Therapeutic Agents:

The present disclosure provides for the use of the hIL 12 muteins of the present disclosure in combination with one or more additional active anti-neoplastic agents (“supplementary agents”) for the treatment of neoplastic disease. Such further combinations are referred to interchangeably as “supplementary anti-neoplastic combinations” or “supplementary anti-neoplastic combination therapy” and those therapeutic agents that are used in combination with hIL 12 muteins of the present disclosure are referred to as “supplementary anti-neoplastic agents.” As used herein, the term “supplementary anti-neoplastic agents” includes anti-neoplastic agents that can be administered or introduced separately, for example, formulated separately for separate administration (e.g., as may be provided in a kit) and/or therapies that can be administered or introduced in combination with the hIL 12 muteins.

### Chemotherapeutic Agents:

In some embodiments, the supplementary anti-neoplastic agent is a chemotherapeutic agent. In some embodiments the supplementary agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A2, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4 (5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary anti-neoplastic agent is one or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foreoing as practied in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the hIL 12 mutein is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11: S115), IDO inhibitors such as epacadostat, and oncolytic viruses such as talimogene laherparepvec (T-VEC).

### Anti-Tumor Antigen Antibody Therapeutics as Supplementary Agents

In some embodiments, a “supplementary anti-neoplastic agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TrikE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFR (e.g. olartumumab), EGFR (e.g. cetuximab, panitumumab and necitumumab), ERBB2 (e.g. trastuzumab), ERBB3, MET, IGFIR, EPHA3, TRAIL R1, TRAIL R2, RANKL RAP, tenascin, integrin V 3, and integrin 4 1.

In some embodiments, a therapeutic antibody is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” In one embodiment, the immune checkpoint pathway modulator is an antagonist of a negative immune checkpoint pathway that inhibits the binding of PD1 to PDL1 and/or PDL2 (“PD1 pathway inhibitor). The term PD1 pathway inhibitors includes monoclonal antibodies that interfere with the binding of PD1 to PDL1 and/or PDL2. Examples of commercially available PD1 pathway inhibitors useful as supplementary agents in the treatment of neoplastic disease include antibodies that interfere with the binding of PD1 to PDL1 and/or PDL2 including but not limited to nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

Examples of antibody therapeutics which are FDA approved and may be used as supplementary agents for use in the treatment of neoplastic disease include atezolizumab, olaratumab, ixekizumab, trastuzumab, infliximab, rituximab, edrecolomab, daratumumab, elotuzumab, necitumumab, dinutuximab, nivolumab, blinatumomab, pembrolizumab, pertuzumab, brentuximab vedotin, ipilimumab, ofatumumab, certolizumab pegol, catumaxomab, panitumumab, bevacizumab, ramucirumab, siltuximab, enfortumab vedotin, polatuzumab vedotin, [fam]-trastuzumab deruxtecan, cemiplimab, moxetumomab pasudotox, mogamuizumab, tildrakizumab, ibalizumab, durvalumab, inotuzumab, ozogamicin, avelumab, obinutuzumab, ado-trastuzumab emtansine, cetuximab, tositumomab-1131, ibritumomab tiuxetan, gemtuzumab, and ozogamicin.

### Physical Methods

In some embodiments, a supplementary anti-neoplastic agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising a hIL 12 mutein and one or more supplementary anti-neoplastic agents. In some embodiments, the present disclosure further contemplates the use of a hIL 12 mutein in combination with surgery (e.g. tumor resection). In some embodiments, the present disclosure further contemplates the use of a hIL 12 mutein in combination with bone marrow transplantation, peripheral blood stem cell transplantation or other types of transplantation therapy.

In some embodiments, the methods of the disclosure may include the combination of the administration of a hIL 12 muteins with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells.

CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3 (4): 388-398; Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15. Examples of commercially available CAR-T cell products include axicabtagene ciloleucel (marketed as Yescarta® commercially available from Gilead Pharmaceuticals) and tisagenlecleucel (marketed as Kymriah® commercially available from Novartis). In some embodiments, the CAR-T possesses a CAR specifically binds to a cell surface molecule associated with a tumor cell is selected from the group consisting of GD2, BCMA, CD19, CD33, CD38, CD70, GD2, IL3RD2, CD19, mesothelin, Her2, EpCam, Muc1, ROR1, CD133, CEA, EGRFRVIII, PSCA, GPC3, Pan-ErbB and FAP.

### Physical Methods:

In some embodiments, the supplementary anti-neoplastic agent is a anti-neoplastic physical methods including but not limited to radiotherapy, cryotherapy, hyperthermic therapy, surgery, laser ablation, and proton therapy.

Methods for Modulating hIL-12 Signaling

In another aspect, the disclosure provides methods for modulating IL-12 mediated signaling in a subject. In some embodiments, the method comprises administering to the subject an effective amount of a pharmaceutical composition to the subject, where the pharmaceutical composition comprises a hIL-12 mutein comprising a modified hIL-12p40 polypeptide described herein, a nucleic acid molecule encoding a modified hIL-12p40 polypeptide described herein, a nucleic acid molecule encoding a p35 polypeptide and a modified hIL-12p40 polypeptide described herein, or a recombinantly modified cell comprising a nucleic acid molecule encoding a modified hIL-12p40 polypeptide (optionally further comprising a nucleic acid encoding a p35 polypeptide) described herein. In some embodiments, the pharmaceutical composition comprises a pharmaceutically acceptable carrier.

In some embodiments, the method for modulating IL-12-mediated signaling in a subject comprises determining STAT4-mediated signaling in one or more cells obtained from the subject. In some embodiments, the STAT4-mediated signaling is determined by an assay selected from the group consisting of a gene expression assay, a phospho-flow signaling assay, and an enzyme-linked immunosorbent assay (ELISA). In some embodiments, the STAT4-mediated signaling in the subject is reduced by about 20% to about 100% compared to a reference level. In some embodiments, the administered composition results in a reduced capacity to induce expression of IFN-γ.

Disorders amenable to treatment with a hIL-13 mutein comprising a modified hIL-12p40 polypeptide (or a nucleic acid encoding a modified hIL-12p40 polypeptide, including recombinant viruses encoding the modified hIL-12p40 polypeptide) of the present disclosure include inflammatory or autoimmune diseases including but not limited to, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome (Seronegativity, Enthesopathy, Arthropathy Syndrome).

Other examples of proliferative and/or differentiative disorders amenable to treatment with a hIL-13 mutein comprising a modified hIL-12p40 polypeptide (or a nucleic acid encoding a modified hIL-12p40 polypeptide, including recombinant viruses encoding the modified hIL-12p40 polypeptide) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of inflammatory or autoimmune skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising a hIL-13 mutein comprising a modified hIL-12p40 polypeptide and/or the nucleic acid molecules that encode modified hIL-12p40 polypeptides including recombinant viruses encoding such modified hIL-12p40 polypeptides) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

### Kits

Also provided are kits comprising the modified hIL-12p40 polypeptides of the disclosure. In some embodiments, the kit comprises one or more components for modulating IL-12-mediated signaling in a subject, or treating a health condition in a subject in need thereof, wherein the components are selected from a hIL-12 mutein comprising a modified hIL-12p40 polypeptide described herein, a modified hIL-12p40 polypeptide described herein, a nucleic acid molecule encoding a modified hIL-12p40 polypeptide described herein, a nucleic acid molecule encoding a p35 polypeptide and a modified hIL-12p40 polypeptide described herein, a recombinantly modified cell comprising a nucleic acid molecule encoding a modified hIL-12p40 polypeptide described herein (optionally further comprising a nucleic acid encoding a p35 polypeptide), or a pharmaceutical composition comprising one of more of the components.

In some embodiments, the kit comprises one or more components for modulating IL-23-mediated signaling in a subject, or treating a health condition in a subject in need thereof, wherein the components are selected from a hIL-23 mutein comprising a modified hIL-12p40 polypeptide described herein, a nucleic acid molecule encoding a modified hIL-12p40 polypeptide described herein, a nucleic acid molecule encoding a human p19 polypeptide and a modified hIL-12p40 polypeptide described herein, a recombinantly modified cell comprising a nucleic acid molecule encoding a modified hIL-12p40 polypeptide described herein (optionally further comprising a nucleic acid encoding a human p19 polypeptide), or a pharmaceutical composition comprising one of more of the components. In some embodiments, the pharmaceutical composition of the kits described herein comprise a pharmaceutically acceptable carrier.

### Examples

The following Examples are provided to illustrate, but not to limit the claimed invention.

### Construction of E81X and F82X Mutant Panel

Two mutant panels were constructed using standard mutagenesis techniques known in the art. In one panel, E81 of hIL-12p40 was mutated to A, and F82 was mutated to all twenty amino acids to create the F82X panel. In another panel, F82 of hIL-12p40 was mutated to A, and E81 was mutated to all twenty amino acids to create the E81X panel. The nucleic acid and amino acid sequences for each of the mutants is set forth in Table 7.

### Generation of the Human IL12 Mammalian Expression Vectors

A pCDNA3.4 mammalian expression vector (Life Technologies, Carlsbad, CA) was modified to include additional restriction sites in the Multiple Cloning Cloning Site (MCS), and renamed pExSyn2.0. The human IL 12 p40 Open Reading Frame (ORF) was cloned into pExSyn2.0 at the EcoRI and BamHI restriction sites, using standard molecular biology cloning techniques. The vector was named “pExSyn2.0-human IL12 p40”. The ORF of the human IL 12 p35 subunit was cloned into pExSyn2.0 at the EcoRI and BamHI sites and incorporated a C-terminal “GS” Linker and an 8X Histidine tag, using standard molecular biology cloning techniques. The resultant vector was named “pExSyn2.0-human IL 12 p35-His”. The vectors were DNA sequenced (MC Lab, South San Francisco, CA) to confirm identity.

Expression of human IL12 Mutants in Expi293 Expression System

The pExSyn2.0-human IL12 p40 and pExSyn2.0-human IL12 p35-His expression vectors were cotransfected into Expi293 Cells following the manufacturer's recommended protocol (Life Technologies, Carlsbad, CA), with the following exceptions: a ratio of 2:1 (p35: p40) DNA was used in the transfections, and 80% of the recommended Enhancer and feed were added at day 1 post transfection. The cultures were harvested when the viability was ˜60-70%.

Purification of Human IL12 Conditioned Media from Expi293 Expression System

His-tagged IL-12 muteins (His-tag on p35 C-terminus) from 4 ml of conditioned media were captured using 0.1 ml Ni Sepharose excel resin (Cytiva, part #GE17371201), equilibrated in Phosphate Buffered Saline (PBS) containing 10 mM Imidazole. The muteins were eluted from the Ni resin with 0.5 ml of PBS containing 250 mM Imidazole and dialyzed into PBS. Concentrations were determined with UV absorbance at 280 nm using extinction coefficients determined from the protein sequence.

HEK-Blue Human IL12 pSTAT4 Reporter Assay

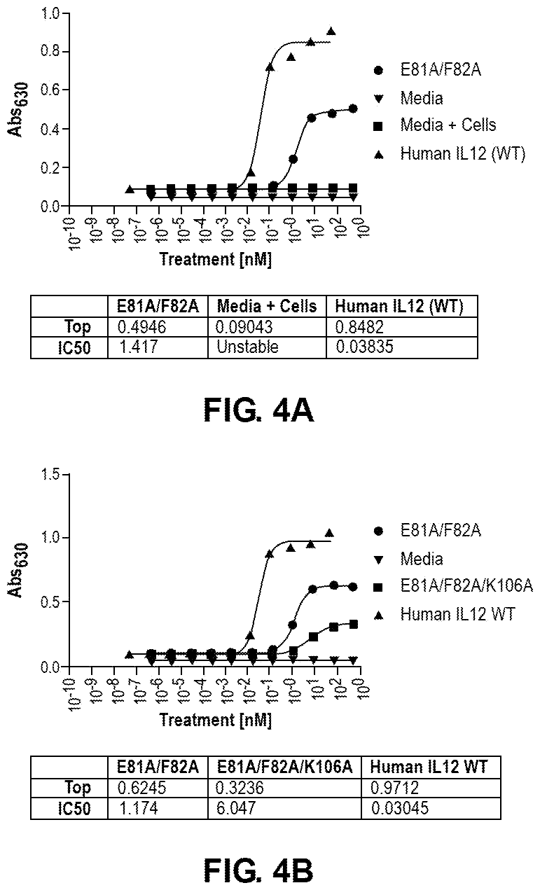

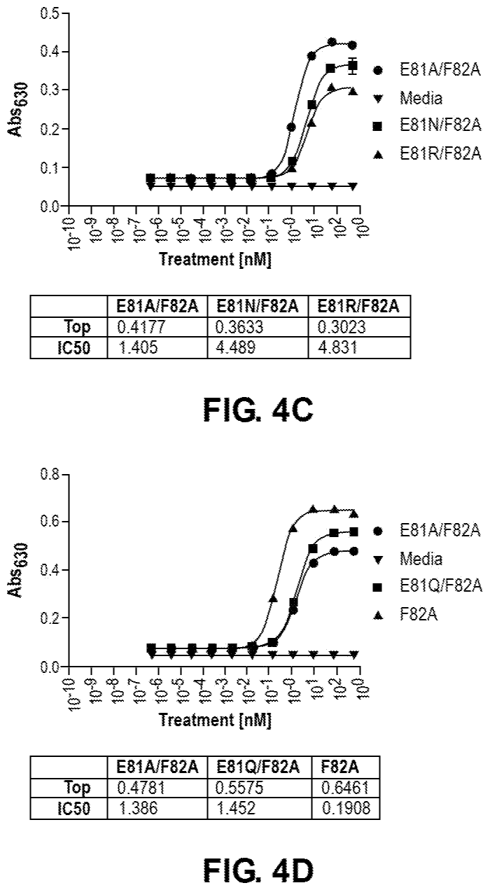

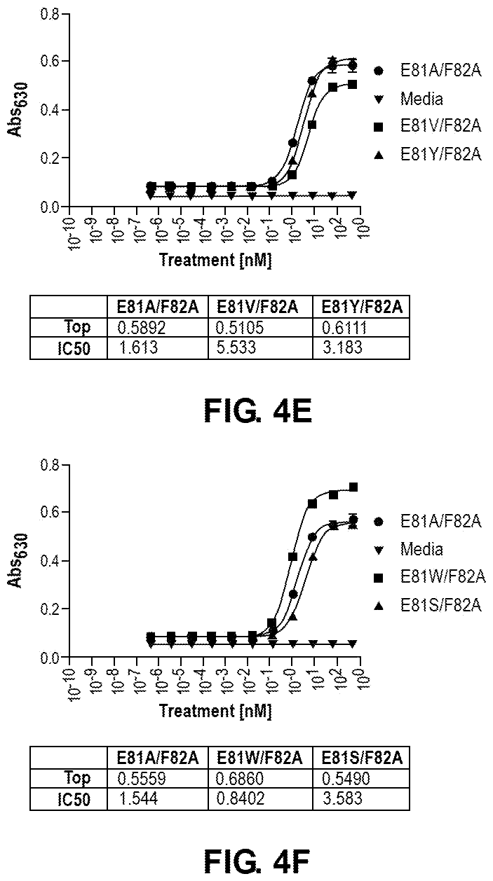

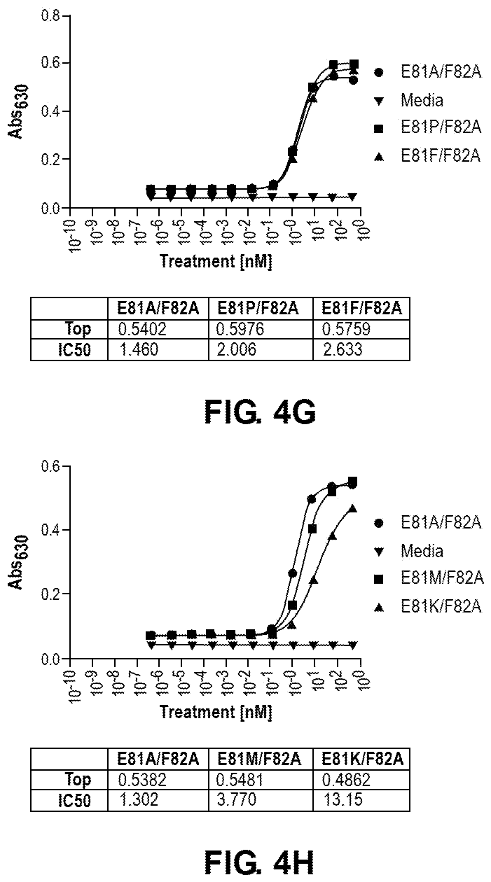

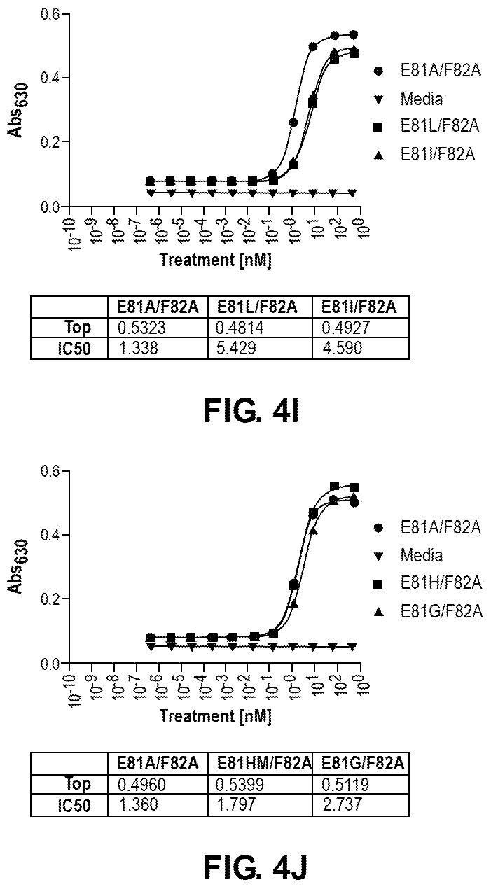

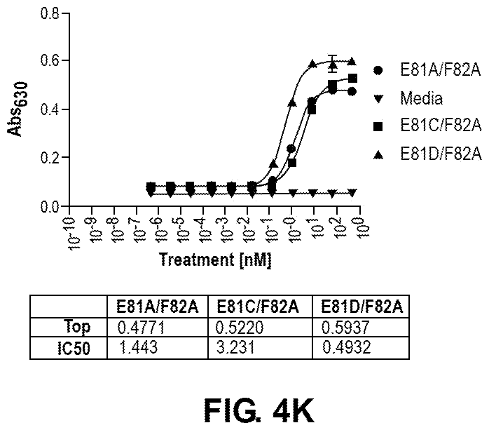

To characterize the mutations' effects on pSTAT4 signaling, the Human IL 12 E81-Shuffle/F82A Series was run the the HEK-Blue Human IL12 pSTAT4 Reporter Assay (Invivogen, San Diego, CA) (It is noted that E81-Shuffle/F82A refers to a series of mutant IL 12 proteins in which numerous amino acids were substituted at position 81, each in combination with the F82A substitution). The manufacturer's protocol was followed with the following notes: The proteins were serially diluted in concentrations ranging from 500 nM to 4.7E-7 nM. The results of the pSTAT4 Reporter Assay are shown in FIGS. 4A-K. All muteins tested showed reduced pSTAT4 activity relative to wild type human IL 12 as judged by increased EC50.

E81x Mutant Panel Methods (IFNγ assay)

Isolated human Whole PBMCs were removed from storage in liquid nitrogen, thawed, and counted. Cells were divided into two groups from which were isolated either Pan-T Cells or Natural Killer Cells using StemCell negative isolation kits (StemCell Technologies, Cat. #17951, Cat. #19055), per manufacturer's protocol. Cells were then counted, resuspended in Complete Yssel's media (IMDM, Gibco, Cat. #122440-053) containing 0.25% w/v Human Albumin (Sigma, Cat. #A9080), 1x ITS-X (human) (Gibco, Cat. #51500056), 30 mg/L Transferrin (Roche, Cat. #10652202001), 2 mg/L PA BioXtra (Sigma, Cat. #P5585), LA-OA-Albumin (Sigma, Cat. #L9655), 1× Penicillin/Streptomycin (Gibco, Cat. #15-140-122), 1% Human Serum (Gemini, Cat. #507533011), and transferred to wells of a 96 well, flat-bottom, tissue-culture treated plate (Fisher Scientific, Cat. #FB012931). The plates used to stimulate Pan-T Cells were coated with 5 μg/mL anti-CD3 antibody (Biolegend, Cat. #300458) in Phosphate Buffered Saline (PBS) (Corning, Cat. #12-031-CV), overnight at 4C, and washed prior to cell isolations. All cells were supplemented with human IL-2 (Synthekine, Lot. #P19029PL1) and recombinant human IL-18 (R&D Systems, Cat. #9124-IL-050/CF), final concentrations 100 pM and 50 ng/ml, respectively. Pan-T Cells were additionally supplemented with 10 μg/mL anti-CD28 antibody (Biolegend, Cat. #302934), final concentration 10 μg/mL. IL-12 mutant proteins were titrated in Complete Yssel's Media at concentrations ranging from 200 nM to 2fM, 1:10 dilutions, and added to wells in equivalent volume to previously plated cells, final concentrations ranging from 100 nM to 1 fM. Cells were then incubated at 37C, 5% CO2 for 48 hours.

In the last 4 hours of incubation, cells were treated with 1:1000 Monensin (eBiosciences, Cat. #00-4505-51). After incubation, cells were washed with PBS and stained with Zombie NIR fixable viability dye (Biolegend, Cat. #423105) for 15 minutes at 4C in the dark. Cells were washed twice in pre-made FACS Buffer (BD, 554656) and then fixed in 1X Phosflow Fix Buffer I (BD, Cat. #557870), pre-heated to 37C, for 10 minutes at 37C, 5% CO2. Cells were then washed with FACS Buffer twice and permeabilized in Phosflow Perm Buffer III (BD, Cat. #558050), per manufacturer's recommendation. After permeabilization, cells were washed twice in FACS Buffer, briefly blocked with 1:10 Human TruStain FcX Fc Block (Biolegend, Cat. #422302) in FACS Buffer and then stained for with anti-IFNγ antibody (Biolegend, Cat. #506507), anti-CD4 antibody (BD, Cat. #552838), anti-CD8 antibody (BD, Cat. #563677), and anti-CD56 antibody (Biolegend, Cat. #362504) for 1 hour at room temperature, in the dark. Cells were then washed with FACS Buffer twice and resuspended in FACS Buffer containing 1% PFA (Electron Microscopy Sciences, Cat. #15710) for at least 10 minutes at 40C in the dark prior to acquisition via flow cytometry.

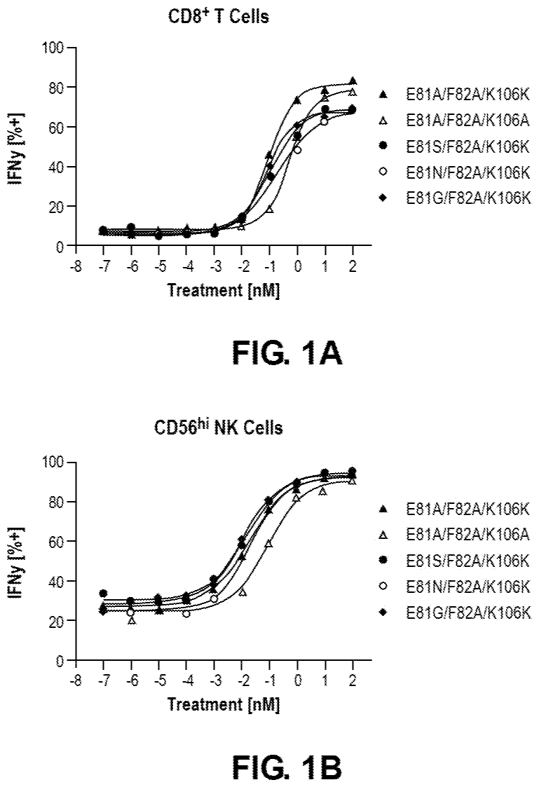

Tables 8 and 9 shows the results of the IFN γ assay used to assess IL-12 signaling in CD8+ T cells and NK cells (C56hi NK cells). FIGS. 1A and 1B show IFNγ production by representative hIL-12p40 mutants (E81A/F82A/K106K; E81S/F82A/K106K; E81N/F82A/K106K, E81G/F82A/K106K of the E81X panel, as compared to E81A/F82A/K106A). All muteins tested were more potent inducers of IFNγ on CD8 and CD56hi cell populations relative to human IL12 E81A/F82A/K106A.

F82x Mutant Panel Methods (pSTAT4 Assay)

Human Whole PBMCs were isolated from Leukoreduction System Chambers (Stanford Blood Center) using the Erythrocyte Custom Sedimentation Kit (Miltenyi Biotec, Cat. #130-126-357) followed by the Custom Buffy Coat/LRSC PBMC Isolation kit (Miltenyi Biotec, Cat. #130-126-448), per manufacturer's protocol. These negatively selected PBMCs were washed in warm Complete Yssel's media (IMDM, Gibco, Cat. #122440-053) containing 0.25% w/v Human Albumin (Sigma, Cat. #A9080), 1x ITS-X (human) (Gibco, Cat. #51500056), 30 mg/L Transferrin (Roche, Cat. #10652202001), 2 mg/L PA BioXtra (Sigma, Cat. #P5585), LA-OA-Albumin (Sigma, Cat. #L9655), 1× Penicillin/Streptomycin (Gibco, Cat. #15-140-122), 1% Human Serum (Gemini, Cat. #507533011), counted, and transferred to a T175 tissue-culture treated flask (Nunc, Cat. #159910) at a concentration of 2E06 cells per mL. Media was supplemented with 1 μg/mL anti-CD3 antibody (Biolegend, Cat. #300458) and 1 μg/mL anti-CD28 antibody (Biolegend, Cat. #302934). Cells were incubated for 72 hours at 37C, 5% CO2.

After incubation, cells were decanted from the flask, washed twice with warm Complete Yssel's media, and allowed to rest at 37C, 5% CO2 without anti-CD3 or anti-CD28 stimulation. Loosely attached cells were detached with gentle washing and manual agitation prior to washes and rest. After resting, cells were washed with PBS and stained with Zombie NIR fixable viability dye (Biolegend, Cat. #423105) for 15 minutes at 4C in the dark. Cells were washed twice in pre-made Assay Buffer (0.5% BSA PBS) and transferred to wells of a 96 well, round-bottom, tissue-culture treated plate (Thermo Scientific, Cat. #163320) and allowed to equilibrate in a 37C, 5% CO2 incubator. After equilibration, cells were treated with an equivalent volume of 2x IL-12 mutant protein diluted in Assay Buffer at 1:10 titrations, final concentrations ranging from 1 uM to 100fM. Cells were then incubated at 37C, 5% CO2 for 15 minutes.

After incubation, cells were fixed in an equivalent volume of pre-warmed, 1X Phosflow Lyse/Fix Buffer (BD, Cat. #558049) for 10 minutes at 37C, 5% CO2. Cells were then washed in Assay Buffer twice and permeabilized in BD Phosflow Perm Buffer III (BD, Cat. #558050), per manufacturer's recommendation. After permeabilization, cells were washed twice in FACS Buffer, briefly blocked with 1:10 Human TruStain FcX Fc Block (Biolegend, Cat. #422302) in FACS Buffer and then stained for with anti-pSTAT4 antibody (CD, Cat. #562703), anti-CD4 antibody (BD, Cat. #552838), anti-CD8 antibody (BD, Cat. #563677), anti-CD3 (Biolegend, Cat. #300415), and anti-CD56 antibody (Biolegend, Cat. #362504) for 1 hour at room temperature, in the dark. Cells were then washed with FACS Buffer twice and resuspended in FACS Buffer containing 1% PFA (Electron Microscopy Sciences, Cat. #15710) for at least 10 minutes at 4C in the dark prior to acquisition via flow cytometry.

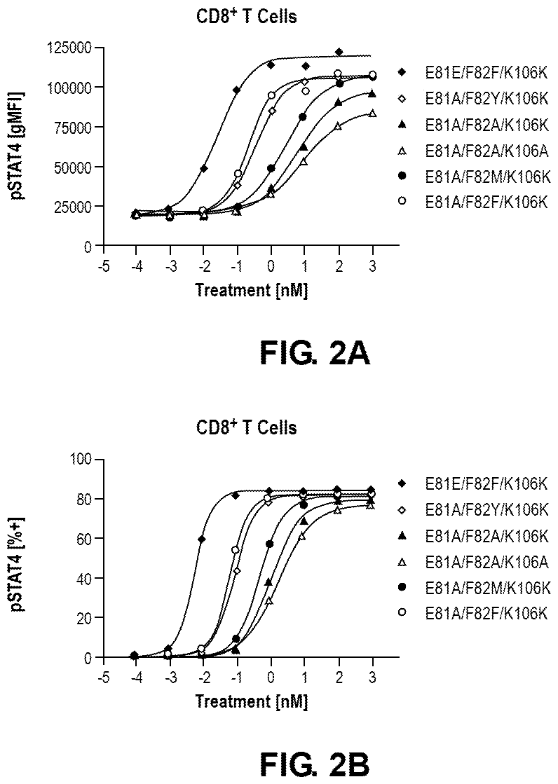

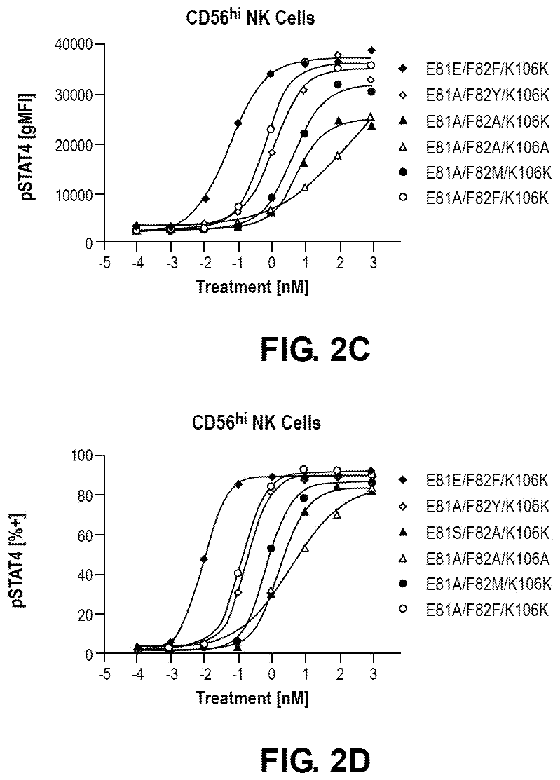

Tables 10 and 11 shows the results of the STAT4 γ assay used to assess IL-12 signaling in CD8+ T cells and NK cells (C56hi NK cells). FIGS. 2A-2D show STAT4 activity by representative hIL-12p40 mutants (E81A/F82Y/K106K; E81A/F82A/K106K; E81A/F82A/K106K; E81A/F82M/K106K; E81A/F82F/K106K) of the F82X panel in CD8+T cells (FIGS. 2A-2B) and NK cells (FIGS. 2C-2D), as compared to E81E/F82F/K106K (wildtype). All muteins tested were less potent inducers of pSTAT4 on CD8 and CD56hi cell populations relative to human IL12 wild type.

All patents, patent applications and publications referred to throughout the disclosure herein are incorporated by reference in their entirety

TABLE 7
Wildtype hIL-12 p40, wildtype hIL-12p35 and mutant hIL-12p40 sequences

| SEQ ID | Description: Human IL12 p35 Subunit, Wild Type |
|---|---|
| No.: | Vector Name: 002 pExSyn2.0-hIL 12p35 |
| DNA | ATGTGCCCTGCCAGATCTCTGCTGCTGGTGGCTACACTGGTGCTGCTGGATCATCTGAGCCTGGC |
| | CAGAAACCTGCCAGTGGCCACGCCTGATCCTGGCATGTTTCCTTGTCTGCACCACAGCCAGAACC |
| | TGCTGAGAGCCGTGTCCAACATGCTGCAGAAGGCCAGACAGACCCTCGAGTTCTACCCCTGCACC |
| | AGCGAGGAAATCGACCACGAGGACATCACCAAGGACAAGACCAGCACCGTGGAAGCCTGCCTGCC |
| | TCTGGAACTGACCAAGAACGAGAGCTGCCTGAACAGCAGAGAGACAAGCTTCATCACCAACGGCT |
| | CTTGCCTGGCCTCCAGAAAGACCTCCTTCATGATGGCCCTGTGCCTGAGCAGCATCTACGAGGAC |
| | CTGAAGATGTACCAGGTCGAGTTCAAGACCATGAACGCCAAGCTGCTGATGGACCCCAAGCGGCA |
| | GATCTTCCTGGACCAGAATATGCTGGCCGTGATCGACGAGCTGATGCAGGCCCTGAACTTCAACA |
| | GCGAGACAGTGCCCCAGAAGTCCAGCCTGGAAGAACCCGACTTCTACAAGACCAAGATCAAGCTG |
| | TGCATCCTGCTGCACGCCTTCCGGATCAGAGCCGTGACCATCGACAGAGTGATGAGCTACCTGAA |
| | CGCCAGCTGATAA |
| PROTEIN | MCPARSLLLVATLVLLDHLSLARNLPVATPDPGMFPCLHHSQNLLRAVSNMLQKARQTLEFYPCT |
| | SEEIDHEDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEPDFYKTKIKL |
| | CILLHAFRIRAVTIDRVMSYLNAS (SEQ ID NO: 3) |
| | Description: Human IL12 p35 (C-terminal 8X His Tag) |
| SEQ ID | Subunit, Wild Type |
| No.: | Vector Name: 001 pExSyn2.0-hIL12p35-8X His |
| DNA | ATGTGCCCTGCCAGATCTCTGCTGCTGGTGGCTACACTGGTGCTGCTGGATCATCTGAGCCTGGC |
| | CAGAAACCTGCCAGTGGCCACGCCTGATCCTGGCATGTTTCCTTGTCTGCACCACAGCCAGAACC |
| | TGCTGAGAGCCGTGTCCAACATGCTGCAGAAGGCCAGACAGACCCTCGAGTTCTACCCCTGCACC |
| | AGCGAGGAAATCGACCACGAGGACATCACCAAGGACAAGACCAGCACCGTGGAAGCCTGCCTGCC |
| | TCTGGAACTGACCAAGAACGAGAGCTGCCTGAACAGCAGAGAGACAAGCTTCATCACCAACGGCT |
| | CTTGCCTGGCCTCCAGAAAGACCTCCTTCATGATGGCCCTGTGCCTGAGCAGCATCTACGAGGAC |
| | CTGAAGATGTACCAGGTCGAGTTCAAGACCATGAACGCCAAGCTGCTGATGGACCCCAAGCGGCA |
| | GATCTTCCTGGACCAGAATATGCTGGCCGTGATCGACGAGCTGATGCAGGCCCTGAACTTCAACA |
| | GCGAGACAGTGCCCCAGAAGTCCAGCCTGGAAGAACCCGACTTCTACAAGACCAAGATCAAGCTG |
| | TGCATCCTGCTGCACGCCTTCCGGATCAGAGCCGTGACCATCGACAGAGTGATGAGCTACCTGAA |
| | CGCCAGCGGATCCCACCATCACCATCACCACCATCATTAGTGA |
| PROTEIN | MCPARSLLLVATLVLLDHLSLARNLPVATPDPGMFPCLHHSQNLLRAVSNMLQKARQTLEFYPCT |
| | SEEIDHEDITKDKTSTVEACLPLELTKNESCLNSRETSFITNGSCLASRKTSFMMALCLSSIYED |
| | LKMYQVEFKTMNAKLLMDPKRQIFLDQNMLAVIDELMQALNFNSETVPQKSSLEEPDFYKTKIKL |
| | CILLHAFRIRAVTIDRVMSYLNASGSHHHHHHHH |
| SEQ ID | Description: Human IL12 p40 Subunit, Wild Type |
| No.: | Vector Name: 004 PCR-pExSyn2.0-hIL 12p40 |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACCATCCAAGTGAAAGAGTTTGGCGACGCCGGCCA |
| | GTACACCTGTCACAAAGGCGGAGAAGTGCTGAGCCACAGCCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA |
| PROTEIN | MCHQQLVISWFSLVFLASPLVAIWELKKDVYVVELDWYPDAPGEMVVLTCDTPEEDGITWTLDQS |
| | SEVLGSGKTLTIQVKEFGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 1) |
| SEQ ID | Description: Human IL12 p40 E81A, F82A |
| No.: | Vector Name: 011 pExSyn2.0-hIL12 p40 E81A, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 47) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 7) |
| MATURE PROTEIN | IWELKKDVYVVELDWYPDAPGEMVVLTCDTPEEDGITWTLDQSSEVLGSGKTLTIQVKAAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 151) |
| SEQ ID | Description: Human IL12 p40 E81A, F82R |
| No.: | Vector Name: 012 pExSyn2.0-hIL12 p40 E81A, F82R |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcccgTGGCGACGCCGGCCA |
| | GTACACaTGTCACAAAGGCGGAGAgGTGCTGAGCCACAGCTGCTGCTGCTCCACAAGAAAGAGGA |
| | TGGCATTTGGAGCACCGACATCCTGAAGGACCAGAAAGAGCCCAAGAACAAGACCTTCCTGAGAT |
| | GCGAGGCCAAGAACTACAGCGGCCGGTTCACATGTTGGTGGCTGACCACCATCAGCACCGACCTG |
| | ACCTTCAGCGTGAAGTCCAGCAGAGGCAGCAGTGATCCTCAGGGCGTTACATGTGGCGCTGCCAC |
| | ACTGTCTGCCGAAAGAGTGCGGGGCGACAACAAAGAATACGAGTACAGCGTGGAATGCCAAGAGG |
| | ACAGCGCCTGTCCAGCCGCCGAAGAGTCTCTGCCTATCGAAGTGATGGTGGACGCCGTGCACAAG |
| | CTGAAGTACGAGAACTACACCTCCAGCTTTTTCATCCGGGACATCATCAAGCCCGATCCTCCAAA |
| | GAACCTGCAGCTGAAGCCTCTGAAGAACAGCAGACAGGTGGAAGTGTCCTGGGAGTACCCCGACA |
| | CCTGGTCTACACCCCACAGCTACTTCAGCCTGACCTTTTGCGTGCAAGTGCAGGGCAAGTCCAAG |
| | CGCGAGAAAAAGGACCGGGTGTTCACCGACAAGACCAGCGCCACCGTGATCTGCAGAAAGAACGC |
| | CAGCATCAGCGTCAGAGCCCAGGATCGGTACTACAGCAGCTCTTGGAGCGAGTGGGCCAGCGTGC |
| | CATGTAGCTGATAA (SEQ ID NO: 48) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKARGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 8) |
| MATURE PROTEIN | IWELKKDVYVVELDWYPDAPGEMVVLTCDTPEEDGITWTLDQSSEVLGSGKTLTIQVKARGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 152) |
| SEQ ID | Description: Human IL12 p40 E81A, F82N |
| No.: | Vector Name: 013 pExSyn2.0-hIL12 p40 E81A, F82N |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccaaTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 49) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKANGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 9) |
| MATURE PROTEIN | IWELKKDVYVVELDWYPDAPGEMVVLTCDTPEEDGITWTLDQSSEVLGSGKTLTIQVKANGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 153) |
| SEQ ID | Description: Human IL12 p40 E81A, F82D |
| No.: | Vector Name: 014 pExSyn2.0-hIL12 p40 E81A, F82D |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccgATGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 50) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKADGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 10) |
| MATURE PROTEIN | IWELKKDVYVVELDWYPDAPGEMVVLTCDTPEEDGITWTLDQSSEVLGSGKTLTIQVKADGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 154) |
| SEQ ID | Description: Human IL12 p40 E81A, F82C |
| No.: | Vector Name: 015 pExSyn2.0-hIL12 p40 E81A, F82C |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcctgTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 51) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKACGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 11) |
| MATURE PROTEIN | IWELKKDVYVVELDWYPDAPGEMVVLTCDTPEEDGITWTLDQSSEVLGSGKTLTIQVKACGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 155) |
| SEQ ID | Description: Human IL12 p40 E81A, F82E |
| No.: | Vector Name: 016 pExSyn2.0-hIL12 p40 E81A, F82E |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccgagGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 52) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAEGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 12) |
| MATURE PROTEIN | IWELKKDVYVVELDWYPDAPGEMVVLTCDTPEEDGITWTLDQSSEVLGSGKTLTIQVKAEGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 156) |
| SEQ ID | Description: Human IL12 p40 E81A, F82Q |
| No.: | Vector Name: 017 pExSyn2.0-hIL12 p40 E81A, F82Q |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcccagGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 53) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAQGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 13) |
| MATURE PROTEIN | IWELKKDVYVVELDWYPDAPGEMVVLTCDTPEEDGITWTLDQSSEVLGSGKTLTIQVKAQGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 157) |
| SEQ ID | Description: Human IL12 p40 E81A, F82G |
| No.: | Vector Name: 018 pExSyn2.0-hIL12 p40 E81A, F82G |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccggTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 54) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAGGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 14) |
| MATURE PROTEIN | IWELKKDVYVVELDWYPDAPGEMVVLTCDTPEEDGITWTLDQSSEVLGSGKTLTIQVKAGGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 158) |
| SEQ ID | Description: Human IL12 p40 E81A, F82H |
| No.: | Vector Name: 019 pExSyn2.0-hIL12 p40 E81A, F82H |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcccaTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 55) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAHGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 15) |
| MATURE PROTEIN | IWELKKDVYVVELDWYPDAPGEMVVLTCDTPEEDGITWTLDQSSEVLGSGKTLTIQVKAHGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 159) |
| SEQ ID | Description: Human IL12 p40 E81A, F82I |
| No.: | Vector Name: 020 pExSyn2.0-hIL12 p40 E81A, F82I |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccatTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 56) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAIGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 16) |
| MATURE PROTEIN | IWELKKDVYVVELDWYPDAPGEMVVLTCDTPEEDGITWTLDQSSEVLGSGKTLTIQVKAIGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 160) |
| SEQ ID | Description: Human IL12 p40 E81A, F82L |
| No.: | Vector Name: 021 pExSyn2.0-hIL12 p40 E81A, F82L |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccctTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 57) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKALGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 17) |
| MATURE PROTEIN | IWELKKDVYVVELDWYPDAPGEMVVLTCDTPEEDGITWTLDQSSEVLGSGKTLTIQVKALGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 161) |
| SEQ ID | Description: Human IL12 p40 E81A, F82K |
| No.: | Vector Name: 022 pExSyn2.0-hIL12 p40 E81A, F82K |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccaaaGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 58) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAKGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 18) |
| MATURE PROTEIN | IWELKKDVYVVELDWYPDAPGEMVVLTCDTPEEDGITWTLDQSSEVLGSGKTLTIQVKAKGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 162) |
| SEQ ID | Description: Human IL12 p40 E81A, F82M |
| No.: | Vector Name: 023 pExSyn2.0-hIL12 p40 E81A, F82M |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccatgGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 59) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAMGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 19) |
| MATURE PROTEIN | IWELKKDVYVVELDWYPDAPGEMVVLTCDTPEEDGITWTLDQSSEVLGSGKTLTIQVKAMGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 163) |
| SEQ ID | Description: Human IL12 p40 E81A, F82F |
| No.: | Vector Name: 024 pExSyn2.0-hIL12 p40 E81A, F82F |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcctttGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 60) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAFGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 20) |
| MATURE PROTEIN | IWELKKDVYVVELDWYPDAPGEMVVLTCDTPEEDGITWTLDQSSEVLGSGKTLTIQVKAFGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 164) |
| SEQ ID | Description: Human IL12 p40 E81A, F82P |
| No.: | Vector Name: 025 pExSyn2.0-hIL12 p40 E81A, F82P |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccccTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 61) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAPGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 21) |
| MATURE PROTEIN | IWELKKDVYVVELDWYPDAPGEMVVLTCDTPEEDGITWTLDQSSEVLGSGKTLTIQVKAPGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 165) |
| SEQ ID | Description: Human IL12 p40 E81A, F82S |
| No.: | Vector Name: 026 pExSyn2.0-hIL12 p40 E81A, F82S |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccagTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 62) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKASGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 22) |
| MATURE PROTEIN | IWELKKDVYVVELDWYPDAPGEMVVLTCDTPEEDGITWTLDQSSEVLGSGKTLTIQVKASGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 166) |
| SEQ ID | Description: Human IL12 p40 E81A, F82T |
| No.: | Vector Name: 027 pExSyn2.0-hIL12 p40 E81A, F82T |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccacTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 63) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKATGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 23) |
| MATURE PROTEIN | IWELKKDVYVVELDWYPDAPGEMVVLTCDTPEEDGITWTLDQSSEVLGSGKTLTIQVKATGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWS |
| | EWASVPCS (SEQ ID NO: 167) |
| SEQ ID | Description: Human IL12 p40 E81A, F82W |
| No.: | Vector Name: 028 pExSyn2.0-hIL12 p40 E81A, F82W |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcctggGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 64) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAWGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 24) |
| MATURE PROTEIN | IWELKKDVYVVELDWYPDAPGEMVVLTCDTPEEDGITWTLDQSSEVLGSGKTLTIQVKAWGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 168) |
| SEQ ID | Description: Human IL12 p40 E81A, F82Y |
| No.: | Vector Name: 029 pExSyn2.0-hIL12 p40 E81A, F82Y |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGcctaTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 65) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAYGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 25) |
| MATURE PROTEIN | IWELKKDVYVVELDWYPDAPGEMVVLTCDTPEEDGITWTLDQSSEVLGSGKTLTIQVKAYGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 169) |
| SEQ ID | Description: Human IL12 p40 E81A, F82V |
| No.: | Vector Name: 030 pExSyn2.0-hIL12 p40 E81A, F82V |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccgtTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 66) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAVGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 26) |
| MATURE PROTEIN | IWELKKDVYVVELDWYPDAPGEMVVLTCDTPEEDGITWTLDQSSEVLGSGKTLTIQVKAVGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 170) |
| SEQ ID | Description: Human IL12 p40 E81R, F82A |
| No.: | Vector Name: 031 pExSyn2.0-hIL12 p40 E81R, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAcgcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 67) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKRAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 27) |
| MATURE PROTEIN | IWELKKDVYVVELDWYPDAPGEMVVLTCDTPEEDGITWTLDQSSEVLGSGKTLTIQVKRAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 171) |
| SEQ ID | Description: Human IL12 p40 E81N, F82A |
| No.: | Vector Name: 032 pExSyn2.0-hIL12 p40 E81N, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAaacgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 68) |
| PROTEIN | MCHQOLVISWFSLVFLASPLVAIWELKKDVYVVELDWYPDAPGEMVVLTCDTPEEDGITWTLDQS |
| | SEVLGSGKTLTIQVKNAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 28) |
| MATURE PROTEIN | IWELKKDVYVVELDWYPDAPGEMVVLTCDTPEEDGITWTLDQSSEVLGSGKTLTIQVKNAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 172) |
| SEQ ID | Description: Human IL12 p40 E81D, F82A |
| No.: | Vector Name: 033 pExSyn2.0-hIL12 p40 E81D, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGacgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 69) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKDAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 29) |
| MATURE PROTEIN | IWELKKDVYVVELDWYPDAPGEMVVLTCDTPEEDGITWTLDQSSEVLGSGKTLTIQVKDAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 173) |
| SEQ ID | Description: Human IL12 p40 E81C, F82A |
| No.: | Vector Name: 034 pExSyn2.0-hIL12 p40 E81C, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAtgcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 70) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKCAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 30) |
| MATURE PROTEIN | IWELKKDVYVVELDWYPDAPGEMVVLTCDTPEEDGITWTLDQSSEVLGSGKTLTIQVKCAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 174) |
| SEQ ID | Description: Human IL12 p40 E81E, F82A |
| No.: | Vector Name: 035 pExSyn2.0-hIL12 p40 E81E, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGAGgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 71) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKEAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 31) |
| MATURE PROTEIN | IWELKKDVYVVELDWYPDAPGEMVVLTCDTPEEDGITWTLDQSSEVLGSGKTLTIQVKEAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 175) |
| SEQ ID | Description: Human IL12 p40 E81Q, F82A |
| No.: | Vector Name: 036 pExSyn2.0-hIL12 p40 E81Q, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAcaggcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 72) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKQAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 32) |
| MATURE PROTEIN | IWELKKDVYVVELDWYPDAPGEMVVLTCDTPEEDGITWTLDQSSEVLGSGKTLTIQVKQAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 176) |
| SEQ ID | Description: Human IL12 p40 E81G, F82A |
| No.: | Vector Name: 037 pExSyn2.0-hIL12 p40 E81G, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGgcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 73) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKGAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 33) |
| MATURE PROTEIN | IWELKKDVYVVELDWYPDAPGEMVVLTCDTPEEDGITWTLDQSSEVLGSGKTLTIQVKGAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 177) |
| SEQ ID | Description: Human IL12 p40 E81H, F82A |
| No.: | Vector Name: 038 pExSyn2.0-hIL12 p40 E81H, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAcacgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 74) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKHAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 34) |
| MATURE PROTEIN | IWELKKDVYVVELDWYPDAPGEMVVLTCDTPEEDGITWTLDQSSEVLGSGKTLTIQVKHAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 178) |
| SEQ ID | Description: Human IL12 p40 E81I, F82A |
| No.: | Vector Name: 039 pExSyn2.0-hIL12 p40 E81I, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAatcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 75) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKIAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 35) |
| MATURE PROTEIN | IWELKKDVYVVELDWYPDAPGEMVVLTCDTPEEDGITWTLDQSSEVLGSGKTLTIQVKIAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 179) |
| SEQ ID | Description: Human IL12 p40 E81L, F82A |
| No.: | Vector Name: 040 pExSyn2.0-hIL12 p40 E81L, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAActcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 76) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKLAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 36) |
| MATURE PROTEIN | IWELKKDVYVVELDWYPDAPGEMVVLTCDTPEEDGITWTLDQSSEVLGSGKTLTIQVKLAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 180) |
| SEQ ID | Description: Human IL12 p40 E81K, F82A |
| No.: | Vector Name: 041 pExSyn2.0-hIL12 p40 E81K, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAaaggcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 77) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKKAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 37) |
| MATURE PROTEIN | IWELKKDVYVVELDWYPDAPGEMVVLTCDTPEEDGITWTLDQSSEVLGSGKTLTIQVKKAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 181) |
| SEQ ID | Description: Human IL12 p40 E81M, F82A |
| No.: | Vector Name: 042 pExSyn2.0-hIL12 p40 E81M, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAatggcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 78) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKMAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 38) |
| MATURE PROTEIN | IWELKKDVYVVELDWYPDAPGEMVVLTCDTPEEDGITWTLDQSSEVLGSGKTLTIQVKMAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 182) |
| SEQ ID | Description: Human IL12 p40 E81F, F82A |
| No.: | Vector Name: 043 pExSyn2.0-hIL12 p40 E81F, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAttcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 79) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKFAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO:39) |
| MATURE PROTEIN | IWELKKDVYVVELDWYPDAPGEMVVLTCDTPEEDGITWTLDQSSEVLGSGKTLTIQVKFAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 183) |
| SEQ ID | Description: Human IL12 p40 E81P, F82A |
| No.: | Vector Name: 044 pExSyn2.0-hIL12 p40 E81P, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAcccgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 80) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKPAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 40) |
| MATURE PROTEIN | IWELKKDVYVVELDWYPDAPGEMVVLTCDTPEEDGITWTLDQSSEVLGSGKTLTIQVKPAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 184) |
| SEQ ID | Description: Human IL12 p40 E81S, F82A |
| No.: | Vector Name: 045 pExSyn2.0-hIL12 p40 E81S, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAagcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 81) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKSAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 41) |
| MATURE PROTEIN | IWELKKDVYVVELDWYPDAPGEMVVLTCDTPEEDGITWTLDQSSEVLGSGKTLTIQVKSAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 185) |
| SEQ ID | Description: Human IL12 p40 E81T, F82A |
| No.: | Vector Name: 046 pExSyn2.0-hIL12 p40 E81T, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAaccgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 82) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKTAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 42) |
| MATURE PROTEIN | IWELKKDVYVVELDWYPDAPGEMVVLTCDTPEEDGITWTLDQSSEVLGSGKTLTIQVKTAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 186) |
| SEQ ID | Description: Human IL12 p40 E81W, F82A |
| No.: | Vector Name: 047 pExSyn2.0-hIL12 p40 E81W, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAtgggcTGGCGACGCCGGCCA |
| | GTACACaTGTCACAAAGGCGGAGAgGTGCTGAGCCACAGCTGCTGCTGCTCCACAAGAAAGAGGA |
| | TGGCATTTGGAGCACCGACATCCTGAAGGACCAGAAAGAGCCCAAGAACAAGACCTTCCTGAGAT |
| | GCGAGGCCAAGAACTACAGCGGCCGGTTCACATGTTGGTGGCTGACCACCATCAGCACCGACCTG |
| | ACCTTCAGCGTGAAGTCCAGCAGAGGCAGCAGTGATCCTCAGGGCGTTACATGTGGCGCTGCCAC |
| | ACTGTCTGCCGAAAGAGTGCGGGGCGACAACAAAGAATACGAGTACAGCGTGGAATGCCAAGAGG |
| | ACAGCGCCTGTCCAGCCGCCGAAGAGTCTCTGCCTATCGAAGTGATGGTGGACGCCGTGCACAAG |
| | CTGAAGTACGAGAACTACACCTCCAGCTTTTTCATCCGGGACATCATCAAGCCCGATCCTCCAAA |
| | GAACCTGCAGCTGAAGCCTCTGAAGAACAGCAGACAGGTGGAAGTGTCCTGGGAGTACCCCGACA |
| | CCTGGTCTACACCCCACAGCTACTTCAGCCTGACCTTTTGCGTGCAAGTGCAGGGCAAGTCCAAG |
| | CGCGAGAAAAAGGACCGGGTGTTCACCGACAAGACCAGCGCCACCGTGATCTGCAGAAAGAACGC |
| | CAGCATCAGCGTCAGAGCCCAGGATCGGTACTACAGCAGCTCTTGGAGCGAGTGGGCCAGCGTGC |
| | CATGTAGCTGATAA (SEQ ID NO: 83) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKWAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 43) |
| MATURE PROTEIN | IWELKKDVYVVELDWYPDAPGEMVVLTCDTPEEDGITWTLDQSSEVLGSGKTLTIQVKWAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 187) |
| SEQ ID | Description: Human IL12 p40 E81Y, F82A |
| No.: | Vector Name: 048 pExSyn2.0-hIL12 p40 E81Y, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAtacgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 84) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKYAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS. (SEQ ID NO: 44) |
| MATURE PROTEIN | IWELKKDVYVVELDWYPDAPGEMVVLTCDTPEEDGITWTLDQSSEVLGSGKTLTIQVKYAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 188) |
| SEQ ID | Description: Human IL12 p40 E81V, F82A |
| No.: | Vector Name: 049 pExSyn2.0-hIL12 p40 E81V, F82A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGtcgcTGGCGACGCCGGCCA |
| | GTACACaTGTCACAAAGGCGGAGAgGTGCTGAGCCACAGtCTGCTGCTGCTCCACAAGAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 85) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKVAGDAGQYTCHKGGEVLSHSLLLLHKKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 45) |
| MATURE PROTEIN | IWELKKDVYVVELDWYPDAPGEMVVLTCDTPEEDGITWTLDQSSEVLGSGKTLTIQVKVAGDAGQ |
| | YTCHKGGEVLSHSLLLLHKKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 189) |
| SEQ ID | Description: Human IL12 p40 E81A, F82A, K106A |
| No.: | Vector Name: 126 pExSyn2-hIL12 p40 E81A, F82A, K106A |
| DNA | ATGTGTCACCAGCAGCTGGTCATCAGCTGGTTCAGCCTGGTGTTCCTGGCCTCTCCTCTGGTGGC |
| | CATCTGGGAGCTGAAGAAAGACGTGTACGTGGTGGAACTGGACTGGTATCCCGATGCTCCTGGCG |
| | AGATGGTGGTGCTGACCTGCGATACCCCTGAAGAGGACGGCATCACCTGGACACTGGATCAGTCT |
| | AGCGAGGTGCTCGGCAGCGGCAAGACCCTGACaATCCAAGTGAAAGccgcTGGCGACGCCGGCCA |
| | GTACACaTGTCACAAAGGCGGAGAgGTGCTGAGCCACAGtCTGCTGCTGCTCCACgccAAAGAGG |
| | ATGGCATTTGGAGCACCGACATCCTGAAGGACCAGAAAGAGCCCAAGAACAAGACCTTCCTGAGA |
| | TGCGAGGCCAAGAACTACAGCGGCCGGTTCACATGTTGGTGGCTGACCACCATCAGCACCGACCT |
| | GACCTTCAGCGTGAAGTCCAGCAGAGGCAGCAGTGATCCTCAGGGCGTTACATGTGGCGCTGCCA |
| | CACTGTCTGCCGAAAGAGTGCGGGGCGACAACAAAGAATACGAGTACAGCGTGGAATGCCAAGAG |
| | GACAGCGCCTGTCCAGCCGCCGAAGAGTCTCTGCCTATCGAAGTGATGGTGGACGCCGTGCACAA |
| | GCTGAAGTACGAGAACTACACCTCCAGCTTTTTCATCCGGGACATCATCAAGCCCGATCCTCCAA |
| | AGAACCTGCAGCTGAAGCCTCTGAAGAACAGCAGACAGGTGGAAGTGTCCTGGGAGTACCCCGAC |
| | ACCTGGTCTACACCCCACAGCTACTTCAGCCTGACCTTTTGCGTGCAAGTGCAGGGCAAGTCCAA |
| | GCGCGAGAAAAAGGACCGGGTGTTCACCGACAAGACCAGCGCCACCGTGATCTGCAGAAAGAACG |
| | CCAGCATCAGCGTCAGAGCCCAGGATCGGTACTACAGCAGCTCTTGGAGCGAGTGGGCCAGCGTG |
| | CCATGTAGCTGATAA (SEQ ID NO: 86) |
| PROTEIN | MCHQQLVISWFSLVFLASPLVAIWELKKDVYVVELDWYPDAPGEMVVLTCDTPEEDGITWTLDQS |
| | SEVLGSGKTLTIQVKAAGDAGQYTCHKGGEVLSHSLLLLHAKEDGIWSTDILKDQKEPKNKTFLR |
| | CEAKNYSGRFTCWWLTTISTDLTFSVKSSRGSSDPQGVTCGAATLSAERVRGDNKEYEYSVECQE |
| | DSACPAAEESLPIEVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQDRYYSSSWSEWASV |
| | PCS (SEQ ID NO: 46) |
| MATURE PROTEIN | IWELKKDVYVVELDWYPDAPGEMVVLTCDTPEEDGITWTLDQSSEVLGSGKTLTIQVKAAGDAGQ |
| | YTCHKGGEVLSHSLLLLHAKEDGIWSTDILKDQKEPKNKTFLRCEAKNYSGRFTCWWLTTISTDL |
| | TFSVKSSRGSSDPQGVTCGAATLSAERVRGDNKEYEYSVECQEDSACPAAEESLPIEVMVDAVHK |
| | LKYENYTSSFFIRDIIKPDPPKNLQLKPLKNSRQVEVSWEYPDTWSTPHSYFSLTFCVQVQGKSK |
| | REKKDRVFTDKTSATVICRKNASISVRAQDRYYSSSWSEWASVPCS (SEQ ID NO: 190) |

Legend
Signal peptide italicized
Mutational changes from wild type in bold and underlined

TABLE 8
E81X panel-IFNγ assay Results

| | IFNy [gMFI] | | | |
|---|---|---|---|---|
| | CD8⁺ T Cells | | CD56^{hi} NK Cells | |
| Mutant | E^{max} | EC⁵⁰ [nM] | E^{max} | EC⁵⁰ [nM] |
| E81R/F82A/K106K | 16275 | 0.35 | 156152 | 0.75 |
| E81K/F82A/K106K | 13204 | 0.51 | 122605 | 0.62 |
| E81S/F82A/K106K | 11341 | 0.21 | 206067 | 0.73 |
| E81N/F82A/K106K | 11436 | 0.43 | 164856 | 0.60 |
| E81G/F82A/K106K | 10699 | 0.14 | 180731 | 0.29 |
| E81V/F82A/K106K | 10637 | 0.38 | 193838 | 0.50 |
| E81L/F82A/K106K | 11489 | 0.39 | 223143 | 0.47 |
| E81I/F82A/K106K | 11667 | 0.55 | 313210 | 1.48 |
| E81A/F82A/K106K | 17105 | 0.41 | 186889 | 2.56 |
| E81A/F82A/K106A | 14960 | 1.40 | 140241 | 2.80 |

TABLE 9
E81X panel-IFNγ assay Results

| IFNy [%+] | |
|---|---|
| CD8⁺ T Cells | CD56^{hi} NK Cells |

| Mutant | E^{max} | EC⁵⁰ [nM] | E^{max} | EC⁵⁰ [nM] |
|---|---|---|---|---|
| E81R/F82A/K106K | 81.57 | 0.178 | 94.03 | 0.036 |
| E81K/F82A/K106K | 78.51 | 0.305 | 93.65 | 0.046 |
| E81S/F82A/K106K | 69.04 | 0.101 | 94.90 | 0.014 |
| E81N/F82A/K106K | 68.20 | 0.195 | 92.75 | 0.022 |
| E81G/F82A/K106K | 67.43 | 0.080 | 93.83 | 0.010 |
| E81V/F82A/K106K | 67.37 | 0.226 | 94.56 | 0.026 |
| E81L/F82A/K106K | 68.02 | 0.185 | 94.85 | 0.003 |
| E81I/F82A/K106K | 68.35 | 0.370 | 93.69 | 0.061 |
| E81A/F82A/K106K | 81.54 | 0.095 | 93.95 | 0.020 |
| E81A/F82A/K106A | 78.37 | 0.533 | 90.82 | 0.079 |

TABLE 10
F82X panel-STAT4 assay Results

| | pSTAT4 [gMFI] | | | |
|---|---|---|---|---|
| | CD8⁺ T Cells | | CD56^{hi} NK Cells | |
| Mutant | E^{max} | EC⁵⁰ [nM] | E^{max} | EC⁵⁰ [nM] |
| E81E/F82F/K106K | 121157 | 0.03 | 37727 | 0.06 |
| E81A/F82F/K106K | 80051 | 6.65 | 21557 | 9.12 |
| E81A/F82P/K106K | 93967 | 2.87 | 29603 | 8.04 |
| E81A/F82S/K106K | 95853 | 3.84 | 26214 | 6.29 |
| E81A/F82T/K106K | 101280 | 4.50 | 29324 | 10.32 |
| E81A/F82W/K106K | 110538 | 1.22 | 30673 | 1.82 |
| E81A/F82Y/K106K | 108463 | 0.37 | 35668 | 1.19 |
| E81A/F82V/K106K | 101529 | 4.33 | 36797 | 17.16 |
| E81A/F82R/K106K | 100795 | 5.54 | 27989 | 7.62 |
| E81A/F82D/K106K | 83985 | 8.72 | 23330 | 15.92 |
| E81A/F82M/K106K | 108984 | 2.64 | 32209 | 4.26 |
| E81A/F82F/K106K | 106860 | 0.20 | 36628 | 0.63 |
| E81A/F82Q/K106K | 91428 | 4.92 | 25860 | 9.87 |
| E81A/F82G/K106K | 98156 | 4.92 | 30343 | 13.46 |
| E81A/F82H/K106K | 107771 | 4.04 | 33538 | 8.57 |
| E81A/F82I/K106K | 106214 | 5.72 | 30503 | 10.41 |
| E81A/F82L/K106K | 100171 | 3.12 | 31061 | 7.21 |
| E81A/F82C/K106K | 87494 | 12.70 | 27039 | 29.97 |
| E81A/F82A/K106K | 99987 | 6.60 | 25047 | 5.94 |
| E81A/F82A/K106A | 86892 | 9.75 | 35726 | 156.30 |

TABLE 11
F82X panel-STAT4 assay Results

| pSTAT4 [%+] | |
|---|---|
| CD8⁺ T Cells | CD56^{hi} NK Cells |

| Mutant | E^{max} | EC⁵⁰ [nM] | E^{max} | EC⁵⁰ [nM] |
|---|---|---|---|---|
| E81E/F82F/K106K | 84.99 | 0.01 | 91.4 | 0.01 |
| E81A/F82F/K106K | 75.94 | 1.86 | 78.25 | 2.27 |
| E81A/F82P/K106K | 79.66 | 0.71 | 87.63 | 1.15 |
| E81A/F82S/K106K | 80.47 | 0.86 | 86.14 | 1.40 |
| E81A/F82T/K106K | 81.17 | 0.87 | 88.18 | 1.90 |
| E81A/F82W/K106K | 84.07 | 0.29 | 88.40 | 0.39 |
| E81A/F82Y/K106K | 83.54 | 0.09 | 91.73 | 0.19 |
| E81A/F82V/K106K | 80.89 | 0.76 | 89.29 | 1.15 |
| E81A/F82R/K106K | 80.21 | 0.98 | 86.94 | 1.36 |
| E81A/F82D/K106K | 76.56 | 2.13 | 84.07 | 3.51 |
| E81A/F82M/K106K | 82.17 | 0.52 | 88.08 | 0.74 |
| E81A/F82F/K106K | 83.27 | 0.07 | 93.47 | 0.13 |
| E81A/F82Q/K106K | 78.55 | 1.08 | 85.10 | 2.05 |
| E81A/F82G/K106K | 80.06 | 1.00 | 87.95 | 1.70 |
| E81A/F82H/K106K | 79.61 | 0.55 | 89.73 | 0.97 |
| E81A/F82I/K106K | 79.18 | 0.67 | 86.02 | 0.90 |
| E81A/F82L/K106K | 80.23 | 0.71 | 89.11 | 1.08 |
| E81A/F82C/K106K | 79.02 | 3.40 | 85.24 | 5.08 |
| E81A/F82A/K106K | 80.16 | 1.13 | 85.18 | 1.85 |
| E81A/F82A/K106A | 77.67 | 1.86 | 86.94 | 4.03 |

| SEQ ID NO: | Description | Amino Acid Sequences |
|---|---|---|
| 140 | IgG1 Lower | DKTHTCPPCP |
| | Hinge | |
| | EU 221-230 | |
| 142 | hIgG1 CH2 | >sp\|P0DOX5\|240-339 |
| | Domain | PSVFLFPPKPKDTLMISRTPEVTCVVVDVSHEDPEVKFN |
| | EU 238-337 | WYVDGVEVHNAKTKPREEQYNSTYRVVSVLTVLHQD |
| | | WLNGKEYKCKVSNKALPAPIEKTIS |
| 143 | wt IgG1 | EPKSC |
| | Upper | |
| | Hinge | |
| | EU 216-220 | |
| 144 | IgG1 Upper | EPKSS |
| | hinge | |
| | w/C220S | |
| 145 | Basic Fc | DKTHTCPPCPAPELLGGPSVFLFPPKPKDTLMISRTPEVT |
| | KNOB | CVVVDVSHEDPEVKFNWYVDGVEVHNAKTKPREEQY |
| | T366W | NSTYRVVSVLTVLHQDWLNGKEYKCKVSNKALPAPIE |
| | (no upper | KTISKAKGQPREPQVYTLPPSRDELTKNQVSLWCLVKG |
| | hinge) | FYPSDIAVEWESNGQPENNYKTTPPVLDSDGSFFLYSKL |
| | IgG1 Fc | TVDKSRWQQGNVFSCSVMHEALHNHYTQKSLSLSPGK |
| | Monomer | |
| | (lower | |
| | hinge/CH2/ | |
| | CH3 | |
| 146 | Basic Fc | DKTHTCPPCPAPELLGGPSVFLFPPKPKDTLMISRTPEVT |
| | HOLE | CVVVDVSHEDPEVKFNWYVDGVEVHNAKTKPREEQY |
| | T366S/L368A/ | NSTYRVVSVLTVLHQDWLNGKEYKCKVSNKALPAPIE |
| | Y407V | KTISKAKGQPREPQVYTLPPSRDELTKNQVSLSCAVKGF |
| | (no Upper | YPSDIAVEWESNGQPENNYKTTPPVLDSDGSFFLVSKLT |
| | hinge) | VDKSRWQQGNVFSCSVMHEALHNHYTQKSLSLSPGK |
| | IgG1 Fc | |
| | Monomer | |
| | (lower | |
| | hinge/CH2/ | |
| | CH3 | |
| 147 | hIgG1 CH3 | >sp\|P0DOX5\|348-444 |
| | Domain | PQVYTLPPSRDELTKNQVSLTCLVKGFYPSDIAVEWESN |
| | EU346-442 | GQPENNYKTTPPVLDSDGSFFLYSKLTVDKSRWQQGNV |
| | | FSCSVMHEALHNHYTQKSLS |

1. A human IL-12p40 (hIL-12p40) polypeptide comprising an amino acid sequence at least 70% identical to SEQ ID NO: 1 comprising two or more amino acid substitutions, wherein the polypeptide comprises amino acid substitutions at positions corresponding to amino acid residues E81 and F82 of SEQ ID NO: 1, wherein
(a) the amino acid substitution at the position corresponding to amino acid residue F82 is F82X, wherein X is any amino acid other than F, and the amino acid substitution at the position corresponding to E81 is selected from the group consisting of asparagine (N), aspartic acid (D), cysteine (C), glutamine (Q), glutamic acid (E), proline (P), tryptophan (W), and tyrosine (Y); or
(b) the amino acid substitution at the position corresponding to amino acid residue E81 is E81X, wherein X is any amino acid other than E and the amino acid substitution at the position corresponding to F82 is selected from the group consisting of arginine (R), asparagine (N), aspartic acid (D), cysteine (C), glutamine (Q), glutamic acid (E), histidine (H), lysine (K), phenylalanine (F) proline (P), tryptophan (W), and tyrosine (Y).

2. The hIL 12p40 polypeptide of claim 1, further comprising one or more amino acid substitutions at one or more positions corresponding to an amino acid residue selected from the group consisting of W37, P39, D40, A41, Q64, K80, A85, E108, D115, H216, K217, L218, and K219 of SEQ ID NO: 1.

3-7. (canceled)

8. A nucleic acid molecule comprising a nucleic acid sequence encoding the hIL 12p40 polypeptide of claim 1.

9. The nucleic acid of claim 8, wherein the nucleic acid molecule encodes a hIL-12 mutein comprising a nucleic acid encoding a hIL-12p40 polypeptide and a hIL-12p35 polypeptide, separated by a nucleic acid encoding a peptide linker.

10-11. (canceled)

12. A cell comprising the hIL 12p40 polypeptide of claim 1.

13. A cell comprising the nucleic acid of claim 8.

14-18. (canceled)

19. A cell culture comprising at least one cell of claim 12 and a culture medium.

20. A method for producing a hIL12 mutein comprising:
a) providing one or more cells of claim 12; and
b) culturing the one or more cells in a culture medium such that the cells produce a hIL 12 mutein comprising the hIL 12p40 polypeptide encoded by the nucleic acid molecule.

21. The method of claim 20, further comprising isolating and/or purifying the produced hIL12 mutein.

22. The method of claim 20, further comprising structurally modifying the produced hIL 12 mutein to increase half-life.

23. The method of claim 22, wherein said modification comprises one or more alterations selected from the group consisting of fusion to a human Fc antibody fragment, fusion to albumin, and PEGylation.

24. (canceled)

25. A pharmaceutical composition comprising a pharmaceutically acceptable carrier and:
a hIL 12 mutein comprising the polypeptide of claim 1.

26-28. (canceled)

29. A method for modulating IL-12-signaling in a subject, the method comprising administering to the subject an effective amount of a composition comprising:
a hIL12 mutein comprising the hIL12p40 polypeptide of claim 1.

30. A method for the treatment of a health condition in a subject in need thereof, the method comprising administering to the subject a composition comprising
the hIL12p40 polypeptide of claim 1.

31. (canceled)

32. The method of claim 31, wherein the subject is a human.

33. The method of claim 29, wherein the subject has been diagnosed with a condition associated with hIL-12p40 mediated signaling.

34. The method of claim 33, wherein the condition is a cancer, an immune disease, or a chronic infection.

35. (canceled)

36. A method for modulating IL-12-signaling in a subject, the method comprising administering to the subject an effective amount of a composition comprising the nucleic acid of claim 8.

37. A method for modulating IL-12-signaling in a subject, the method comprising administering to the subject an effective amount of a composition comprising the cell of claim 17.


SEQUENCE LISTING (190 sequences)

SEQ ID NO: 1 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 22 aa, Synthetic construct
MCHQQLVISWFSLVFLASPLVA

SEQ ID NO: 3 - protein, 219 aa, Synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 22 aa, Synthetic construct
MCPARSLLLVATLVLLDHLSLA

SEQ ID NO: 5 - protein, 189 aa, Synthetic construct
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 6 - protein, 19 aa, Synthetic construct
MLGSRAVMLLLLLPWTAQG

SEQ ID NO: 7 - protein, 327 aa, Synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPC

SEQ ID NO: 8 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKARGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKANGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKADGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKACGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 12 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAEGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAQGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAGGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAHGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 16 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAIGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 17 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKALGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 18 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAKGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 19 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAMGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 20 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 21 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAPGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 22 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKASGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 23 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKATGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAWGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 25 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAYGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 26 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAVGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 27 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKRAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 28 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKNAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKDAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKCAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 31 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 32 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKQAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 33 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKGAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 34 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKHAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 35 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKIAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 36 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKLAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 37 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKKAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 38 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKMAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKFAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKPAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKSAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 42 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKTAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 43 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKWAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 44 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKYAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 45 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKVAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 46 - protein, 328 aa, Synthetic construct
MCHQQLVISWFSLVFLASPLVAIWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 48 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcccgtggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 49 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccaatggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 50 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccgatggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 51 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcctgtggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 52 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccgagggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 53 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcccagggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 54 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccggtggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 55 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcccatggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 56 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccattggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 57 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcccttggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 58 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccaaaggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 59 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccatgggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 60 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcctttggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 61 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcccctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 62 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccagtggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 63 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccactggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 64 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcctggggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 65 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagcctatggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 66 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccgttggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 67 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaacgcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 68 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaaacgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 69 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagacgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 70 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaatgcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 71 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagaggctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 72 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaacaggctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 73 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaggcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 74 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaacacgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 75 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaatcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 76 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaactcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 77 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaaaggctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 78 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaatggctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 79 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaattcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 80 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaacccgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 81 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaagcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 82 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaaaccgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 83 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaatgggctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 84 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaatacgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 85 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagtcgctggcgacgccggccagtacacatgtcacaaaggcggagaggtgctgagccacagtctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 86 - DNA, 990 bp, Synthetic construct
  source 1..990: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgacaatccaagtgaaagccgctggcgacgccggccagtacacatgtcacaaaggcggagaggtgctgagccacagtctgctgctgctccacgccaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgctgccacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggatcggtactacagcagctcttggagcgagtgggccagcgtgccatgtagctgataa

SEQ ID NO: 87 - skipped

SEQ ID NO: 88 - DNA, 12 bp, Synthetic construct
  source 1..12: mol_type = other DNA
gggagggaggga

SEQ ID NO: 89 - DNA, 12 bp, Synthetic construct
  source 1..12: mol_type = other DNA
gggagggggagg

SEQ ID NO: 90 - DNA, 12 bp, Synthetic construct
  source 1..12: mol_type = other DNA
gggsgggsgggs

SEQ ID NO: 91 - DNA, 12 bp, Synthetic construct
  source 1..12: mol_type = other DNA
gggsgggggsgg

SEQ ID NO: 92 - DNA, 10 bp, Synthetic construct
  source 1..10: mol_type = other DNA
ggggagggga

SEQ ID NO: 93 - DNA, 15 bp, Synthetic construct
  source 1..15: mol_type = other DNA
ggggaggggagggga

SEQ ID NO: 94 - DNA, 12 bp, Synthetic construct
  source 1..12: mol_type = other DNA
ggggagggggag

SEQ ID NO: 95 - DNA, 14 bp, Synthetic construct
  source 1..14: mol_type = other DNA
ggggaggggggagg

SEQ ID NO: 96 - DNA, 10 bp, Synthetic construct
  source 1..10: mol_type = other DNA
ggggsggggs

SEQ ID NO: 97 - DNA, 15 bp, Synthetic construct
  source 1..15: mol_type = other DNA
ggggsggggsggggs

SEQ ID NO: 98 - DNA, 12 bp, Synthetic construct
  source 1..12: mol_type = other DNA
ggggsgggggsg

SEQ ID NO: 99 - DNA, 14 bp, Synthetic construct
  source 1..14: mol_type = other DNA
ggggsggggggsgg

SEQ ID NO: 100 - DNA, 14 bp, Synthetic construct
  source 1..14: mol_type = other DNA
gggggaggggggag

SEQ ID NO: 101 - DNA, 14 bp, Synthetic construct
  source 1..14: mol_type = other DNA
gggggsggggggsg

SEQ ID NO: 102-104 - skipped (3 entries)

SEQ ID NO: 105 - DNA, 12 bp, Synthetic construct
  source 1..12: mol_type = other DNA
gggagggsggga

SEQ ID NO: 106-108 - skipped (3 entries)

SEQ ID NO: 109 - DNA, 12 bp, Synthetic construct
  source 1..12: mol_type = other DNA
gggaggggggag

SEQ ID NO: 110 - skipped

SEQ ID NO: 111 - DNA, 12 bp, Synthetic construct
  source 1..12: mol_type = other DNA
gggsgggagggs

SEQ ID NO: 112 - DNA, 12 bp, Synthetic construct
  source 1..12: mol_type = other DNA
gggsggggggsg

SEQ ID NO: 113 - skipped

SEQ ID NO: 114 - DNA, 15 bp, Synthetic construct
  source 1..15: mol_type = other DNA
ggggaggggsgggga

SEQ ID NO: 115 - DNA, 15 bp, Synthetic construct
  source 1..15: mol_type = other DNA
ggggaggggsggggs

SEQ ID NO: 116 - DNA, 11 bp, Synthetic construct
  source 1..11: mol_type = other DNA
ggggaggggag

SEQ ID NO: 117 - DNA, 10 bp, Synthetic construct
  source 1..10: mol_type = other DNA
ggggaggggs

SEQ ID NO: 118 - DNA, 11 bp, Synthetic construct
  source 1..11: mol_type = other DNA
ggggaggggsg

SEQ ID NO: 119 - skipped

SEQ ID NO: 120 - DNA, 12 bp, Synthetic construct
  source 1..12: mol_type = other DNA
ggggagggggsg

SEQ ID NO: 121 - DNA, 12 bp, Synthetic construct
  source 1..12: mol_type = other DNA
ggggagggggag

SEQ ID NO: 122 - DNA, 12 bp, Synthetic construct
  source 1..12: mol_type = other DNA
ggggagggggsg

SEQ ID NO: 123 - skipped

SEQ ID NO: 124 - DNA, 13 bp, Synthetic construct
  source 1..13: mol_type = other DNA
ggggagggggsgg

SEQ ID NO: 125 - skipped

SEQ ID NO: 126 - DNA, 15 bp, Synthetic construct
  source 1..15: mol_type = other DNA
ggggsggggagggga

SEQ ID NO: 127 - DNA, 15 bp, Synthetic construct
  source 1..15: mol_type = other DNA
ggggsggggaggggs

SEQ ID NO: 128 - DNA, 15 bp, Synthetic construct
  source 1..15: mol_type = other DNA
ggggsggggsgggga

SEQ ID NO: 129 - DNA, 10 bp, Synthetic construct
  source 1..10: mol_type = other DNA
ggggsgggga

SEQ ID NO: 130 - DNA, 11 bp, Synthetic construct
  source 1..11: mol_type = other DNA
ggggsggggag

SEQ ID NO: 131 - DNA, 11 bp, Synthetic construct
  source 1..11: mol_type = other DNA
ggggsggggsg

SEQ ID NO: 132 - DNA, 12 bp, Synthetic construct
  source 1..12: mol_type = other DNA
ggggsggggagg

SEQ ID NO: 133 - DNA, 12 bp, Synthetic construct
  source 1..12: mol_type = other DNA
ggggsgggggag

SEQ ID NO: 134 - DNA, 12 bp, Synthetic construct
  source 1..12: mol_type = other DNA
ggggsgggggsg

SEQ ID NO: 135 - DNA, 13 bp, Synthetic construct
  source 1..13: mol_type = other DNA
ggggsggggggsg

SEQ ID NO: 136 - skipped

SEQ ID NO: 137 - DNA, 14 bp, Synthetic construct
  source 1..14: mol_type = other DNA
gggggsgggggagg

SEQ ID NO: 138-139 - skipped (2 entries)

SEQ ID NO: 140 - protein, 9 aa, Synthetic construct
DKTHTCPPC

SEQ ID NO: 141 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 100 aa, Synthetic construct
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 143 - protein, 5 aa, Synthetic construct
EPKSC

SEQ ID NO: 144 - protein, 5 aa, Synthetic construct
EPKSS

SEQ ID NO: 145 - protein, 227 aa, Synthetic construct [sequence omitted]

SEQ ID NO: 146 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 147 - protein, 97 aa, Synthetic construct
PQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLS

SEQ ID NO: 148 - protein, 306 aa, Synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 149 - protein, 197 aa, Synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 150 - protein, 170 aa, Synthetic construct
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 151 - protein, 306 aa, Synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 152 - protein, 306 aa, Synthetic construct
IWELKKDVYVVELDWYPDAPGEMVVLTCDTPEEDGITWTLDQSSEVLGSGKTLTIQVKARGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 153 - protein, 306 aa, Synthetic construct
IWELKKDVYVVELDWYPDAPGEMVVLTCDTPEEDGITWTLDQSSEVLGSGKTLTIQVKANGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 154 - protein, 306 aa, Synthetic construct
IWELKKDVYVVELDWYPDAPGEMVVLTCDTPEEDGITWTLDQSSEVLGSGKTLTIQVKADGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 155 - protein, 306 aa, Synthetic construct
IWELKKDVYVVELDWYPDAPGEMVVLTCDTPEEDGITWTLDQSSEVLGSGKTLTIQVKACGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 156 - protein, 306 aa, Synthetic construct
IWELKKDVYVVELDWYPDAPGEMVVLTCDTPEEDGITWTLDQSSEVLGSGKTLTIQVKAEGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 157 - protein, 306 aa, Synthetic construct
IWELKKDVYVVELDWYPDAPGEMVVLTCDTPEEDGITWTLDQSSEVLGSGKTLTIQVKAQGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 158 - protein, 306 aa, Synthetic construct
IWELKKDVYVVELDWYPDAPGEMVVLTCDTPEEDGITWTLDQSSEVLGSGKTLTIQVKAGGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 159 - protein, 306 aa, Synthetic construct
IWELKKDVYVVELDWYPDAPGEMVVLTCDTPEEDGITWTLDQSSEVLGSGKTLTIQVKAHGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 160 - protein, 306 aa, Synthetic construct
IWELKKDVYVVELDWYPDAPGEMVVLTCDTPEEDGITWTLDQSSEVLGSGKTLTIQVKAIGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 161 - protein, 306 aa, Synthetic construct
IWELKKDVYVVELDWYPDAPGEMVVLTCDTPEEDGITWTLDQSSEVLGSGKTLTIQVKALGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 162 - protein, 233 aa, Synthetic construct
VLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 163 - protein, 306 aa, Synthetic construct
IWELKKDVYVVELDWYPDAPGEMVVLTCDTPEEDGITWTLDQSSEVLGSGKTLTIQVKAMGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 164 - protein, 306 aa, Synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 165 - protein, 306 aa, Synthetic construct
IWELKKDVYVVELDWYPDAPGEMVVLTCDTPEEDGITWTLDQSSEVLGSGKTLTIQVKAPGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 166 - protein, 328 aa, Synthetic construct; identical to SEQ ID NO: 22

SEQ ID NO: 167 - protein, 306 aa, Synthetic construct
IWELKKDVYVVELDWYPDAPGEMVVLTCDTPEEDGITWTLDQSSEVLGSGKTLTIQVKATGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 168 - protein, 306 aa, Synthetic construct
IWELKKDVYVVELDWYPDAPGEMVVLTCDTPEEDGITWTLDQSSEVLGSGKTLTIQVKAWGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 169 - protein, 306 aa, Synthetic construct
IWELKKDVYVVELDWYPDAPGEMVVLTCDTPEEDGITWTLDQSSEVLGSGKTLTIQVKAYGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 170 - protein, 306 aa, Synthetic construct
IWELKKDVYVVELDWYPDAPGEMVVLTCDTPEEDGITWTLDQSSEVLGSGKTLTIQVKAVGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 171 - protein, 306 aa, Synthetic construct
IWELKKDVYVVELDWYPDAPGEMVVLTCDTPEEDGITWTLDQSSEVLGSGKTLTIQVKRAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 172 - protein, 306 aa, Synthetic construct
IWELKKDVYVVELDWYPDAPGEMVVLTCDTPEEDGITWTLDQSSEVLGSGKTLTIQVKNAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 173 - protein, 306 aa, Synthetic construct
IWELKKDVYVVELDWYPDAPGEMVVLTCDTPEEDGITWTLDQSSEVLGSGKTLTIQVKDAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 174 - protein, 306 aa, Synthetic construct
IWELKKDVYVVELDWYPDAPGEMVVLTCDTPEEDGITWTLDQSSEVLGSGKTLTIQVKCAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 175 - protein, 306 aa, Synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 176 - protein, 233 aa, Synthetic construct; identical to SEQ ID NO: 162

SEQ ID NO: 177 - protein, 306 aa, Synthetic construct
IWELKKDVYVVELDWYPDAPGEMVVLTCDTPEEDGITWTLDQSSEVLGSGKTLTIQVKGAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 178 - protein, 306 aa, Synthetic construct
IWELKKDVYVVELDWYPDAPGEMVVLTCDTPEEDGITWTLDQSSEVLGSGKTLTIQVKHAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 179 - protein, 306 aa, Synthetic construct
IWELKKDVYVVELDWYPDAPGEMVVLTCDTPEEDGITWTLDQSSEVLGSGKTLTIQVKIAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 180 - protein, 306 aa, Synthetic construct
IWELKKDVYVVELDWYPDAPGEMVVLTCDTPEEDGITWTLDQSSEVLGSGKTLTIQVKLAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 181 - protein, 306 aa, Synthetic construct
IWELKKDVYVVELDWYPDAPGEMVVLTCDTPEEDGITWTLDQSSEVLGSGKTLTIQVKKAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 182 - protein, 306 aa, Synthetic construct
IWELKKDVYVVELDWYPDAPGEMVVLTCDTPEEDGITWTLDQSSEVLGSGKTLTIQVKMAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 183 - protein, 306 aa, Synthetic construct
IWELKKDVYVVELDWYPDAPGEMVVLTCDTPEEDGITWTLDQSSEVLGSGKTLTIQVKFAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 184 - protein, 306 aa, Synthetic construct
IWELKKDVYVVELDWYPDAPGEMVVLTCDTPEEDGITWTLDQSSEVLGSGKTLTIQVKPAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 185 - protein, 306 aa, Synthetic construct
IWELKKDVYVVELDWYPDAPGEMVVLTCDTPEEDGITWTLDQSSEVLGSGKTLTIQVKSAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 186 - protein, 306 aa, Synthetic construct
IWELKKDVYVVELDWYPDAPGEMVVLTCDTPEEDGITWTLDQSSEVLGSGKTLTIQVKTAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 187 - protein, 306 aa, Synthetic construct
IWELKKDVYVVELDWYPDAPGEMVVLTCDTPEEDGITWTLDQSSEVLGSGKTLTIQVKWAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 188 - protein, 306 aa, Synthetic construct
IWELKKDVYVVELDWYPDAPGEMVVLTCDTPEEDGITWTLDQSSEVLGSGKTLTIQVKYAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 189 - protein, 306 aa, Synthetic construct
IWELKKDVYVVELDWYPDAPGEMVVLTCDTPEEDGITWTLDQSSEVLGSGKTLTIQVKVAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 190 - protein, 306 aa, Synthetic construct
IWELKKDVYVVELDWYPDAPGEMVVLTCDTPEEDGITWTLDQSSEVLGSGKTLTIQVKAAGDAGQYTCHKGGEVLSHSLLLLHAKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS